(19) United States
(12) Patent Application Publication
(10) Pub. No.: US 2015/0045556 A1
(43) Pub. Date: Feb. 12, 2015

(54) PROCESSES FOR THE PREPARATION OF PERIPHERAL OPIOID ANTAGONIST COMPOUNDS AND INTERMEDIATES THERETO

(71) Applicants: Roland E. Dolle, Ashland, MA (US); Bertrand Le Bourdonnec, Northborough, MA (US); Pierre Martin, Rheinfelden (CH); Christian Steffen Moessner, Lorrach (DE); Felix Herbert Spindler, Starrkirch-Wil (CH); Dirk Jost Spielvogel, Lorrach (DE)

(72) Inventors: Roland E. Dolle, Ashland, MA (US); Bertrand Le Bourdonnec, Northborough, MA (US); Pierre Martin, Rheinfelden (CH); Christian Steffen Moessner, Lorrach (DE); Felix Herbert Spindler, Starrkirch-Wil (CH); Dirk Jost Spielvogel, Lorrach (DE)

(21) Appl. No.: 14/233,670

(22) PCT Filed: Jul. 17, 2012
(86) PCT No.: PCT/US2012/047082

§ 371 Date: Oct. 15, 2014

Related U.S. Application Data
(60) Provisional application No. 61/508,817, filed on Jul. 18, 2011.

(51) Int. Cl. C07D 211/22

(52) U.S. Cl. CPC C07D 211/22
USPC 546/239

(57) ABSTRACT

Novel processes for the preparation of peripheral opioid antagonist compounds and intermediates thereto. The compounds prepared by the present processes may be useful, for example, as antagonists to the mu, kappa and delta opioid receptors, and thereby may be useful in the treatment of gastrointestinal motility disorders, and in preventing peripheral opiate induced side effects. The present processes may offer improved yields, chemical or stereochemical purity, ease of preparation and/or isolation of intermediates and final product, and more industrially useful reaction conditions and workability.

### CROSS REFERENCE TO RELATED APPLICATIONS

This application is a national stage entry of International Application No. PCT/US2012/047082, filed Jul. 17, 2012, which claims priority to and the benefit under 35 U.S.C. §119(e) of U.S. Provisional Application No. 61/508,817, filed Jul. 18, 2011, both of which are hereby incorporated by reference in their entireties.

### FIELD OF THE INVENTION

The invention relates to novel processes for the preparation of peripheral opioid antagonist compounds, as well as intermediates thereof. The present processes may offer improved yields, chemical or stereochemical purity, ease of preparation and/or isolation of intermediates and final product, and more industrially useful reaction conditions and workability.

### BACKGROUND OF THE INVENTION

The trans-3,4-dimethyl-4-(3-hydroxyphenyl)piperidines are an important class of compounds which exhibit opioid antagonist activity as a result of the 3-methyl substituent. Alvimopan™ (i.e., (+)-2-[(S)-benzyl-3-[4(R)-(3-hydroxyphenyl)-3(R),4-dimethylpiperidin-1-yl]propionamidolacetic acid), shown below in a zwitterionic form, represents an example of this class of opioid antagonists. This compound is a peripherally-active antagonist which has a high affinity for the μ-opioid receptor in the lining of the gastrointestinal tract and is useful in the treatment of gastrointestinal motility disorders. See, e.g., U.S. Pat. Nos. 5,270,328; 5,250,542; 5,159,081; and 5,434,171, the contents of which are all incorporated by reference in their entireties.

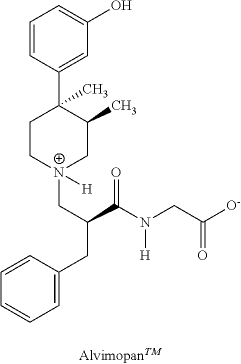 [SMILES: C[C@H]1C[NH+](C[C@H](Cc2ccccc2)C(=O)NCC(=O)[O-])CC[C@@]1(C)c1cccc(O)c1]

A synthesis of Alvimopan™ has been described in Werner et al., J. Org. Chem., 1996, 61, 587. The drug product was prepared in 12 steps and 6.2% yield from 1,3-dimethyl-4-piperidone as starting material. The synthesis includes the preparation of a (3R,4R)-3,4-dimethyl-4-(3-hydroxyphenyl)-piperidine nucleus (A), which was achieved in seven steps and 14.4% overall yield. While the next step, involving the Michael addition of (A) to methyl acrylate to produce intermediate (B), proceeds in good yield (96%), alkylation of the dianion of (B) with benzyl bromide proceeds with poor diastereoselectivity (47:53 mixture of the (3R,4R,αS)- and (3R,4R,αR)-isomers of the alkylation products, respectively). Consequently, the diastereomers require separation by recrystallization of their hydrochloride salts from methanol, resulting in low yields of intermediate (C) (34%). The poor diastereoselectivity of the alkylation step contributes to the low overall yield for the synthesis of Alvimopan.

A synthesis of Alvimopan has also been described in U.S. Pat. No. 6,794,510 and its related divisional application, U.S. Pat. No. 7,091,354. These processes target the Michael addition/modification of (3R,4R)-3,4-dimethyl-4-(3-hydroxyphenyl)-piperidine nucleus (2) followed by a two-step hydroxyl activation/hydrogenolysis sequence as an alternative to a previous synthesis of Alvimopan™, which reportedly offered improved yields, purity, ease of preparation and/or isolation of intermediates and final product, and more industrially useful reaction conditions and workability as compared to prior art processes.

Wentland (U.S. Pat. No. 7,265,226) discloses certain 1-alkyl-4-(3-substitutedphenyl)piperidines that are reportedly prepared from their 1-alkyl-4-(3-hydroxyphenyl)piperidine precursors. The phenyl substituents reported therein include —C(═O)NH₂, —C(═S)NH₂, —C(═O)NHOH, and —NHCHO.

In view of the importance of Alvimopan and related trans-3,4-dimethyl-4-(3-hydroxyphenyl)piperidine derivatives and intermediates in the treatment of gastrointestinal motility disorders and other conditions involving μ-opioid receptors, improved syntheses are needed. Such improvements may include, for example, one or more of the following: enhanced selectivity of individual reaction steps, increased product yields, use of lower cost starting materials, lowered energy consumption (e.g., avoidance of reactions conducted at very high or low temperatures or pressures), reduction in the number of synthetic steps, improved scale up conditions, and the like. The compounds, methods and compositions of the present invention are directed to these, as well as other important needs.

### SUMMARY OF THE INVENTION

Accordingly, the present invention is directed, in part, to novel processes for preparing Alvimopan™ and related trans-3,4-dimethyl-4-(3-hydroxyphenyl)piperidine derivatives and intermediates thereto.

Specifically, in one aspect, there are provided processes for preparing a compound of Formula Ia, a compound of Formula Ib, or mixture thereof:

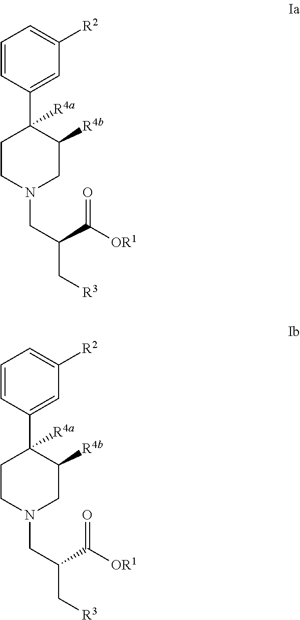

wherein:
each R¹ is independently H, alkyl, or aralkyl;
each R² is independently Cl, Br, I, —C(═O)OR^{5b}, —CN, —═R⁶, or —CONR⁷R⁸;
each R³ is independently H, alkyl, cycloalkyl, or aryl;
each R^{4a} and R^{4b} is independently C₁₋₆alkyl;
each R^{5b} is independently H or alkyl;
each R⁶ is independently H, alkyl, cycloalkyl, alkylcycloalkyl, aralkyl, or an hydroxyl protecting group;
each R⁷ is independently H, alkyl, cycloalkyl, alkylcycloalkyl, or aralkyl; and
each R⁸ is independently H, alkyl, aralkyl, or aryl;
or a salt thereof;
   comprising contacting an N-alkenylpiperidine compound of Formula IIa, Formula IIb, Formula IIc or Formula IId, or mixture thereof:

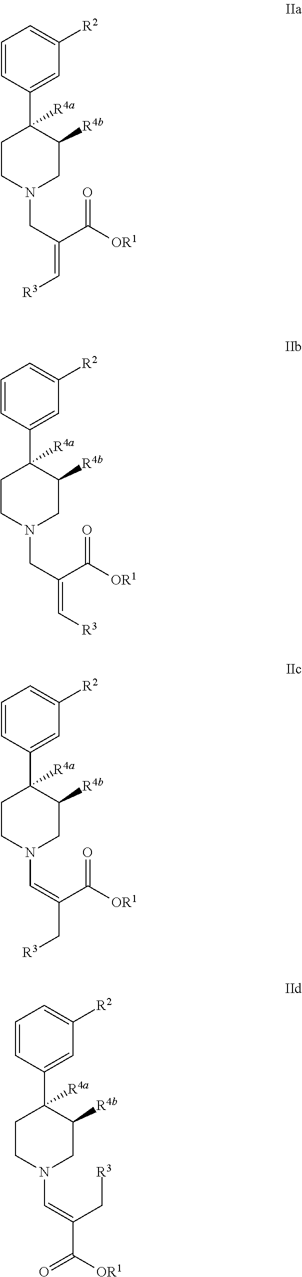

with hydrogen in the presence of a hydrogenation catalyst and a chiral phosphorus-containing ligand for a time and under conditions effective to provide the compound of Formula Ia, the compound of Formula Ib, or mixture thereof.

In one embodiment, the invention provides a process for preparing an N-alkylpiperidine compound of Formula Ia′, or a pharmaceutically acceptable salt thereof:

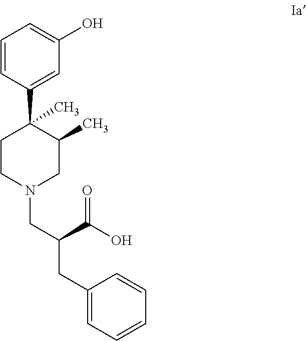 [SMILES: C[C@H]1CN(C[C@H](Cc2ccccc2)C(=O)O)CC[C@@]1(C)c1cccc(O)c1]

comprising contacting an N-alkenylpiperidine compound of Formula IIa′ or Formula IIb′ or mixture thereof:

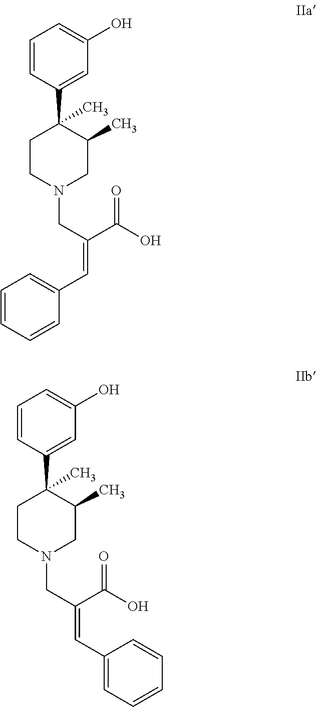 [SMILES: C[C@H]1CN(C/C(=C\c2ccccc2)C(=O)O)CC[C@@]1(C)c1cccc(O)c1 ; C[C@H]1CN(C/C(=C/c2ccccc2)C(=O)O)CC[C@@]1(C)c1cccc(O)c1]

with hydrogen in the presence of a Group VIII transition metal hydrogenation catalyst and a chiral-phosphorous containing ligand for a time and under conditions effective to provide a compound of Formula Ia′.

In a further embodiment, a Compound of Formula Ia′ is is contacted with an alkylting agent to provide a compound of Formula Ia″:

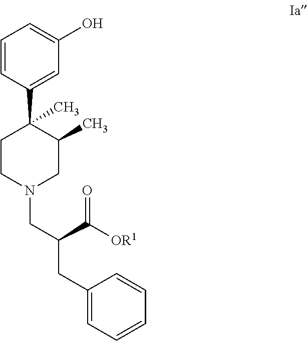

Wherein R¹ is alkyl or aralkyl.

Another aspect of the invention relates to processes for preparing a compound of Formula IIa, a compound of Formula IIb, or mixture thereof:

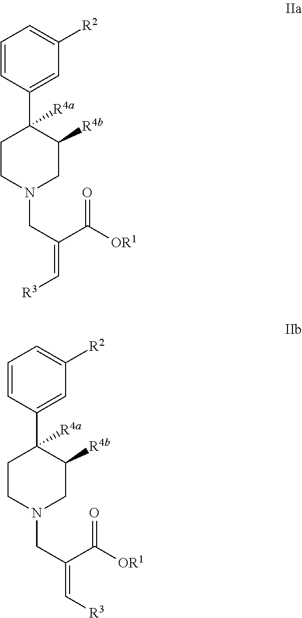

wherein:
each R¹ is independently H, alkyl, or aralkyl;
each R² is independently Cl, Br, I, —C(═O)OR^{5b}, —CN, —OR⁶, or —CONR⁷R⁸;
each R³ is independently H, alkyl, cycloalkyl, or aryl;
each R^{4a} and R^{4b} is independently C₁₋₆alkyl;
each R^{5b} is independently H or alkyl;
each R⁶ is independently H, alkyl, cycloalkyl, alkyl cycloalkyl, aralkyl, or an hydroxyl protecting group;
each R⁷ is independently H, alkyl, cycloalkyl, alkylcycloalkyl, or aralkyl; and
each R⁸ is independently H, alkyl, aralkyl, or aryl;
or a salt thereof;
   comprising contacting a piperidine compound of Formula III:

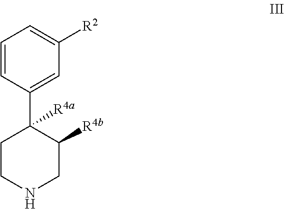

with an alkene compound of Formula IVa or Formula IVb, or mixture thereof:

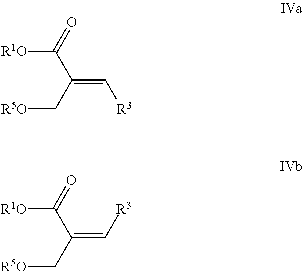

wherein:
each R⁵ is independently alkyl, aralkyl, or —C(═O)R^{5a}; and
each R^{5a} is independently H, alkyl, or aralkyl;
   for a time and under conditions effective to provide the N-alkenylpiperidine compound of Formula IIa, Formula IIb, or mixture thereof.

Still another aspect relates to processes for preparing a compound of Formula Va or Formula Vb, or mixture thereof:

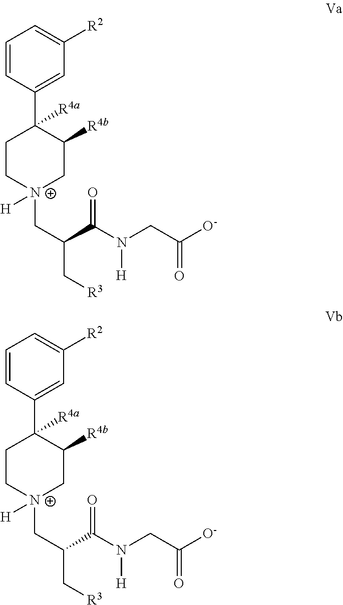

wherein:
each R² is independently Cl, Br, I, —C(═O)OR^{5b}, —CN, —OR⁶, or —CONR⁷R⁸;
each R³ is independently H, alkyl, cycloalkyl, or aryl;
each R^{4a} and R^{4b} is independently C₁₋₆alkyl;
each R^{5b} is independently H or alkyl;
each R⁶ is independently H, alkyl, cycloalkyl, alkylcycloalkyl, aralkyl, or an hydroxyl protecting group;
each R⁷ is independently H, alkyl, cycloalkyl, alkylcycloalkyl, or aralkyl; and
each R⁸ is independently H, alkyl, aralkyl, or aryl;
or a salt thereof;
   comprising providing an N-alkenylpiperidine compound of Formula IIa, Formula IIb, Formula IIc, or Formula IId, or mixture thereof:

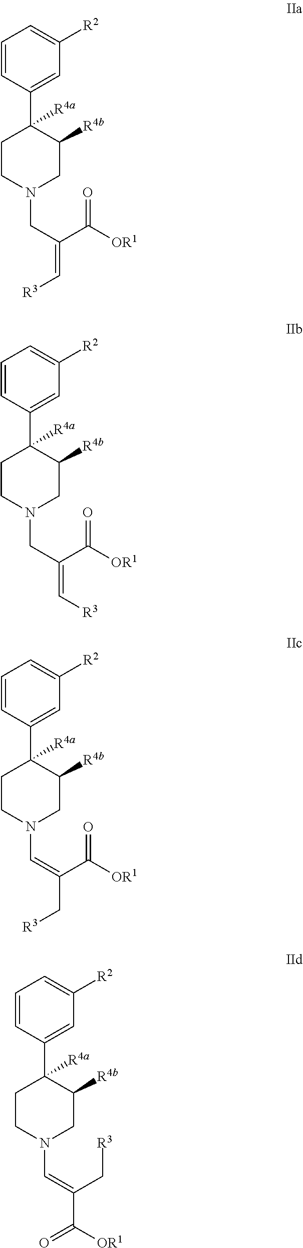

wherein:
R¹ is independently H, alkyl, or aralkyl;
   contacting the N-alkenylpiperidine compound of Formula IIa, Formula IIb, Formula IIc or Formula IId, or mixture thereof, with hydrogen in the presence of a hydrogenation catalyst and a chiral phosphorus-containing ligand for a time and under conditions effective to provide a compound of Formula Ia, a compound of Formula Ib, or mixture thereof:

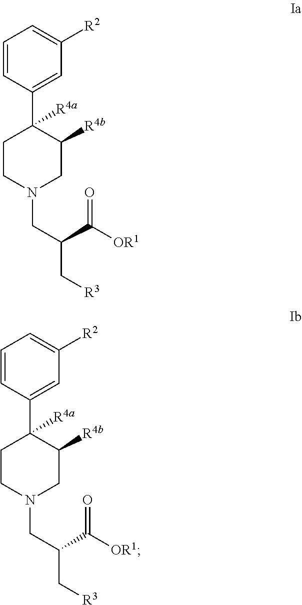

and contacting the compound of Formula Ia, Formula Ib, or mixture thereof, with NH₂CH₂CO₂H for a time and under conditions effective to provide the compound of Formula Va, Formula Vb, or mixture thereof.

In one particular embodiment, a compound of Formula Ia″:

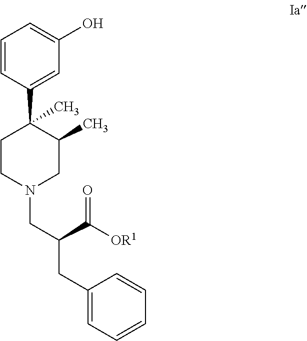

is contacted with NH₂CH₂CO₂H for a time and under conditions effective to provide (+)-2-[(S)-benzyl-3-[4(R)-(3-hydroxyphenyl)-3(R),4-dimethylpiperidin-1-yl]propionamidolacetic acid (alvimopam), or a pharmaceutically acceptable salt thereof.

In another aspect, the invention provides novel chemical compounds. In one such embodiment, the disclosure relates to compounds of Formula IIa:

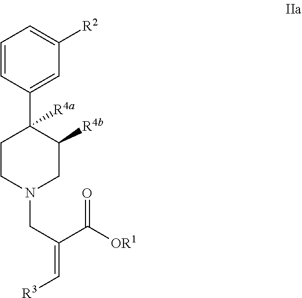

wherein:
R¹ is H, alkyl, or aralkyl;
R² is Cl, Br, I, —C(═O)OR^{5b}, —CN, —OR⁶, or —CONR⁷R⁸;
R³ is H, alkyl, cycloalkyl, or aryl;
R^{4a} and R^{4b} are each independently C₁₋₆alkyl;
R^{5b} is independently H or alkyl;
R⁶ is H, alkyl, cycloalkyl, alkylcycloalkyl, aralkyl, or an hydroxyl protecting group;
R⁷ is H, alkyl, cycloalkyl, alkylcycloalkyl, or aralkyl; and
R⁸ is H, alkyl, aralkyl, or aryl;
   or a salt thereof.

In a particular embodiment, the disclosure provides a compound of Formula IIa′:

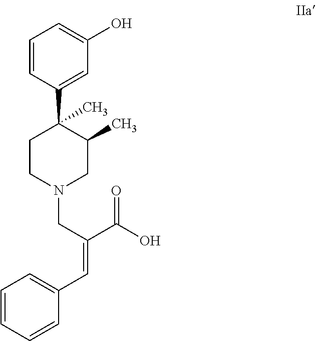 [SMILES: C[C@H]1CN(C/C(=C\c2ccccc2)C(=O)O)CC[C@@]1(C)c1cccc(O)c1]

or a pharmaceutically acceptable salt thereof.

In another particular embodiment, the disclosure provides a compound of Formula IIb′:

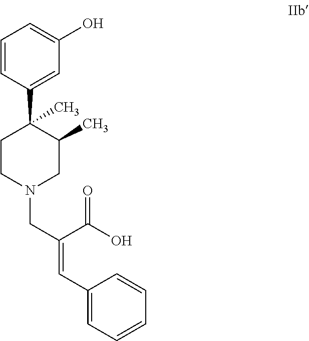 [SMILES: C[C@H]1CN(C/C(=C/c2ccccc2)C(=O)O)CC[C@@]1(C)c1cccc(O)c1]

or a pharmaceutically acceptable salt thereof.

In another embodiment, the disclosure provides compounds compounds of Formula IIb:

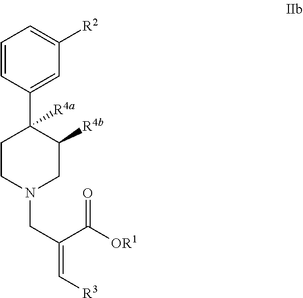

wherein:
R¹ is H, alkyl, or aralkyl;
R² is Cl, Br, I, —C(═O)OR^{5b}, —CN, —OR⁶, or —CONR⁷R⁸;
R³ is H, alkyl, cycloalkyl, or aryl;
R^{4a} and R^{4b} are each independently C₁₋₆alkyl;
R^{5b} is independently H or alkyl;
R⁶ is H, alkyl, cycloalkyl, alkylcycloalkyl, aralkyl, or an hydroxyl protecting group;
R⁷ is H, alkyl, cycloalkyl, alkylcycloalkyl, or aralkyl; and
R⁸ is H, alkyl, aralkyl, or aryl;
   or a salt thereof.

In still another embodiment, the disclosure provides compounds of Formula IIc:

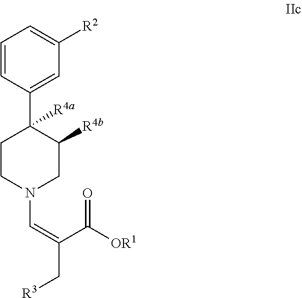

wherein:
R¹ is H, alkyl, or aralkyl;
R² is Cl, Br, I, —C(═O)OR^{5b}, —CN, —OR⁶, or —CONR⁷R⁸;
R³ is H, alkyl, cycloalkyl, or aryl;
R^{4a} and R^{4b} are each independently C₁₋₆alkyl;
R^{5b} is independently H or alkyl;
R⁶ is H, alkyl, cycloalkyl, alkylcycloalkyl, aralkyl, or an hydroxyl protecting group;
R⁷ is H, alkyl, cycloalkyl, alkylcycloalkyl, or aralkyl; and
R⁸ is H, alkyl, aralkyl, or aryl;
   or a salt thereof.

In yet another embodiment, the disclosure provides compounds of Formula IId:

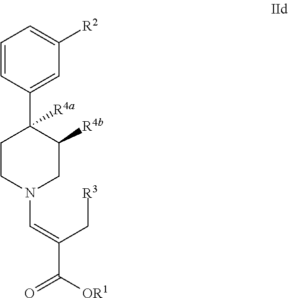

wherein:
R¹ is H, alkyl, or aralkyl;
R² is Cl, Br, I, —C(═O)OR^{5b}, —CN, —OR⁶, or —CONR⁷R⁸;
R³ is H, alkyl, cycloalkyl, or aryl;
R^{4a} and R^{4b} are each independently C₁₋₆alkyl;
R^{5b} is independently H or alkyl;
R⁶ is H, alkyl, cycloalkyl, alkylcycloalkyl, aralkyl, or an hydroxyl protecting group;
R⁷ is H, alkyl, cycloalkyl, alkylcycloalkyl, or aralkyl; and
R⁸ is H, alkyl, aralkyl, or aryl;
   or a salt thereof.

### BRIEF DESCRIPTION OF THE DRAWINGS

FIG. 1 shows a scheme for the synthesis of Alvimopan according to an embodiment of the invention.

### DETAILED DESCRIPTION OF PREFERRED EMBODIMENTS

As used herein, the term “contacting” refers to the bringing together of compounds to within distances that allow for intermolecular interactions and chemical transformations accompanying such interactions. Often, contacting compounds are in solution phase.

As used herein, “alkyl” refers to a saturated straight, branched, or cyclic hydrocarbon, preferably straight or branched hydrocarbon, having from about 1 to about 20 carbon atoms (and all combinations and subcombinations of ranges and specific numbers of carbon atoms therein), preferably from about 1 to about 8, more preferably from about 1 to about 6, with from about 1 to about 4 carbon atoms being more preferred. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl. Alkyl groups can be substituted or unsubstituted.

As used herein, “alkenyl” refers to an alkyl group having one or more double bonds.

As used herein, “alkynyl” refers to an alkyl group having one or more triple bonds.

As used herein, “aryl” refers to a mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl. Aryl groups can be substituted or unsubstituted.

As used herein, “aralkyl” refers to aryl-substituted alkyl radicals having from about 6 to about 50 carbon atoms (and all combinations and subcombinations of ranges and specific numbers of carbon atoms therein), with from about 6 to about 10 carbon atoms being preferred. Non-limiting examples include, for example, benzyl, diphenylmethyl, triphenylmethyl, phenylethyl, and diphenylethyl. Aralkyl groups can be substituted or unsubstituted.

As used herein, “heteroaryl” refers to a mono-, di-, tri-, or other multicyclic aromatic ring system that includes at least one, and preferably from 1 to about 4 sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl. Heteroaryl groups can be substituted or unsubstituted.

As used herein, “heterocyclyl” refers to a mono-, di-, tri-, or other multicyclic aliphatic ring system that includes at least one, and preferably from 1 to about 4 sulfur, oxygen, or nitrogen heteroatom ring members. Heterocyclyl groups can have from about 3 to about 20 carbon atoms (and all combinations and subcombinations of ranges and specific numbers of carbon atoms therein), with from about 4 to about 10 carbons being preferred. The heterocyclyl group can also comprise unsaturations, and can also be fused to aromatic rings. Examples of heterocyclyl groups include, for example, tetrahydrofuranyl, tetrahydrothienyl, piperidinyl, pyrrolidinyl, isoxazolidinyl, isothiazolidinyl, pyrazolidinyl, oxazolidinyl, thiazolidinyl, and imidazolidinyl. Heterocyclyl groups can be substituted or unsubstituted.

As used herein, “silyl” refers to a group having the formula SiR′₃ where each R′ is, independently, H, alkyl, aryl, aralkyl.

As used herein, “alkylcarbonyl” refers to an alkyl—C(═O)— group.

As used herein, “arylcarbonyl” refers to an aryl—C(═O)— group.

As used herein, “aralkylcarbonyl” refers to an aralkyl—C(═O)— group.

As used herein, “heteroarylcarbonyl” refers to a heteroaryl—C(═O)— group.

As used herein, “heterocyclylcarbonyl” refers to a heterocyclyl—C(═O)— group.

As used herein, “carboxyl” refers to a —C(═O)—OH group.

Typically, substituted chemical moieties include one or more substituents that replace hydrogen. Exemplary substituents include, for example, halo (e.g., F, Cl, Br, I), alkyl, alkenyl, alkynyl, aralkyl, aryl, heteroaryl, heterocyclyl, hydroxyl (OH), nitro (NO₂), cyano (CN), cyanato (CNO), thiocyanato (SCN), amino (e.g., NH₃, NHR″, NR″₂), azido (N₃), carboxyl (COOH), C(O)R″, OR″, C(O)OR″, NHC(O)R″, aminocarbonyl, thiol, thiolato (SR″), sulfonic acid (SO₃H), phosphonic acid (PO₃H), SO₂R″, phosphino (PH₂, PHR″, PR″₂), silyl (SiR″₃, SiHR″₂, SiH₂R″, SiH₃) and the like. In relation to the aforementioned substituents, each moiety R″ can be, independently, any of H, alkyl, aryl, aralkyl, heteroaryl, or heterocyclyl, for example.

As used herein, the phrase “protecting group” refers to a moiety that renders a chemical functionality of a molecule inert to specific reaction conditions. The protecting group can later be removed from such functionality in a molecule, preferably without altering or substantially altering the remainder of the molecule. Protecting groups are well known in the art and are well described, for example, in Greene, T. W., et al., Protecting Groups in Organic Synthesis 2nd edition, John Wiley and Sons, Inc., New York, (1991), the disclosure of which is incorporated herein by reference in its entirety.

Accordingly, the phrase “hydroxyl protecting group” refers to a chemical moiety that renders a hydroxyl group inert to certain reaction conditions, such as reaction conditions designed to alter or change the molecule containing the hydroxyl group at a location other than the hydroxyl group. Hydroxyl protecting groups typically replace the hydrogen of the hydroxyl group and can be removed under conditions that do not substantially affect the remainder of the molecule. Exemplary hydroxyl protecting groups include, for example, alkyl, aryl, aralkyl, heteroaryl, heterocyclyl, alkylcarbonyl, arylcarbonyl, aralkylcarbonyl, heteroarylcarbonyl, heterocyclylcarbonyl, or silyl groups.

The phrase “activating group” refers to a moiety that renders a chemical functionality more sensitive to modification under certain reaction conditions. For example, an activating group may convert a poor leaving group into a good leaving group or increase (or decrease) susceptibility to nucleophilic attack or other chemical transformations.

Accordingly, the phrase “hydroxyl activating group” refers to a moiety that replaces the hydrogen of the hydroxyl group, thereby altering the chemical and electronic properties of the hydroxyl group such that the hydroxyl group is more susceptible to removal, such as by replacement with hydrogen or a moiety other than a hydroxyl group. Exemplary hydroxyl activating groups include, for example, alkyl, aryl, aralkyl, heteroaryl, heterocyclyl, alkylcarbonyl, arylcarbonyl, aralkylcarbonyl, heteroarylcarbonyl, heterocyclylcarbonyl, C(S)O-aryl, C(S)O-alkyl, or silyl.

As used herein, “side effect” refers to a consequence other than the one(s) for which an agent or measure is used, as the adverse effects produced by a drug, especially on a tissue or organ system other than the one sought to be benefitted by its administration. In the case, for example, of opioids, the term “side effect” may preferably refer to such conditions as, for example, constipation, nausea and/or vomiting.

“Pharmaceutically acceptable” refers to those compounds, materials, compositions, salts and/or dosage forms which, within the scope of sound medical judgment, are suitable for administration to patients without excessive toxicity, irritation, allergic response, or other problems or complications commensurate with a reasonable benefit/risk ratio.

“Salts” refer to derivatives of the disclosed compounds wherein the parent compound is modified by making acid or base salts thereof, or wherein the parent compound is in its zwitterionic form. When contacted with an acid, for example, resulting in the protonation of an amine functionality, the compound becomes associated with an anion, i.e., the counter ion of the acid. When contacted with a base, for example, resulting in the deprotonation of an acid functionality, the compound is associated with a cation, i.e., the counterion of the base. Examples of salts include, but are not limited to, mineral or organic acid salts of basic residues such as amines, alkali or organic base salts of acidic residues such as carboxylic acids, and the like. Suitable mineral or organic acids or bases that may be employed in preparing salts of the compounds of the invention would be readily apparent to one of ordinary skill in the art, once placed in possession of the present application.

In certain preferred embodiments, the salts are “pharmaceutically acceptable salts”, which include, for example, conventional salts derived from pharmaceutically acceptable acids or bases, as well as internal or zwitterionic salts. Such pharmaceutically acceptable salts include those derived from inorganic acids such as hydrochloric, hydrobromic, sulfuric, sulfamic, phosphoric or nitric acid and the like; and salts prepared from organic acids such as acetic, propionic, succinic, glycolic, stearic, lactic, malic, tartaric, citric, ascorbic, pamoic, maleic, hydroxymaleic, phenylacetic, aspartic, glutamic, benzoic, salicylic, sulfanilic, acetoxybenzoic, fumaric, toluenesulfonic, naphthyldisulfonic, methanesulfonic, ethane disulfonic, oxalic or isethionic acid, and the like. Pharmaceutically acceptable salts also include those derived from metal bases, including alkali metal bases, for example, alkali hydroxides such as sodium hydroxide, potassium hydroxide and lithium hydroxide in which the metal is a monovalent species, alkaline earth metal bases, for example, alkaline earth metal hydroxides such as magnesium hydroxide and calcium hydroxide in which the metal is a polyvalent species, basic amines such as, for example, N,N′-dibenzylethylenediamine, arginine, chloroprocaine, choline, diethanolamine, ethylenediamine, meglumine (N-methylglucamine) and procaine, ammonium bases or alkoxides.

Physiologically acceptable salts as described herein may be prepared by methods known in the art, for example, by dissolving the free amine bases with an excess of the acid in aqueous alcohol, or neutralizing a free carboxylic acid with a metal base, preferably an alkali metal base such as a hydroxide, a substituted or unsubstituted ammonium hydroxide, an alkoxide, or an amine In addition, it is well known to ordinarily skilled artisans that in compounds containing, for example, both a basic nitrogen atom and an acidic group, the nitrogen atom and the acidic functionalities may exist in equilibrium with their zwitterionic form depending, for example, on the characteristics of the involved aqueous medium including, for example, its ionic strength, pH, temperature, salts involved when the aqueous medium is in the form of a buffer, and the like. These zwitterionic salts are, in essence, internal pharmaceutically acceptable salts, and are contemplated to be within the scope of the present invention. Thus, any of the compounds described hereinthroughout that contain, for example, both amino and carboxyl groups, also include reference to their corresponding zwitterions. Similarly, any of the compounds described hereinthroughout that are expressed as zwitterions also include reference to their free amino/carboxylic acid forms.

The term “ammonium base”, as used herein, refers to ammonium hydroxide (NH₄OH), as well as substituted ammonium hydroxides, i.e., NR₄OH, where one, two, three or four of the R groups may be, independently, alkyl, cycloalkyl, alkenyl, aryl, aralkyl, heteroaryl, or heterocycloalkyl. Exemplary substituted ammonium hydroxides include, for example, tetraalkyl ammonium hydroxides, such as tetramethyl ammonium hydroxide.

The term “alkoxide”, as used herein, refers to the product from the reaction of an alkyl alcohol with a metal. Exemplary alkoxides include, for example, sodium ethoxide, potassium ethoxide and sodium t-butoxide.

Compounds described herein may be used or prepared in alternate forms. For example, many amino-containing compounds can be used or prepared as acid addition salts. Often such salts improve isolation and handling properties of the compound. The acid employed in forming acid addition salts is not generally limited. Pharmaceutically acceptable and pharmaceutically unacceptable acids may be used to prepare acid addition salts. For example, depending on the reagents, reaction conditions and the like, compounds as described herein can be used or prepared, for example, as their hydrochloride, hydrogen sulfate, sulfate, methanesulfonate, or tosylate salts. Similarly, compounds as described herein can be used or prepared, for example, as their oxalic acid or succinic acid salts, wherein one or both, preferably one, of the carboxylic acid groups in oxalic or succinic acid protonates the basic nitrogen atom in the compound of Formula Ia, Ib, IIa, IIb, IIc, IId, Va, or Vb, preferably the compound of Formula Va or Vb.

Generally speaking, pharmaceutically unacceptable salts are not useful as medicaments in vivo. However, such salts may in certain cases demonstrate improved crystallinity and thus may be useful, for example, in the synthesis of compounds of Formula Ia, Ib, IIa, IIb, IIc, IId, Va, or Vb, preferably the compound of Formula Va or Vb, such as in connection with the formation, isolation and/or purification of compounds of Formula Ia, Ib, IIa, IIb, IIc, IId, Va, or Vb, preferably the compound of Formula Va or Vb, and/or intermediates thereto. This may result, for example, in improved synthesis, purification or formulation by preparing and/or using compounds of the invention as salts that may not typically be considered to be pharmaceutically acceptable salts. These non-pharmaceutically acceptable salts may be prepared from acids or bases that are not typically considered to be pharmaceutically acceptable. Examples of such salts include, for example, acid addition salts prepared from trifluoroacetic acid, perchloric acid and tetrafluoroboric acid. Non-pharmaceutically acceptable salts may be employed in certain embodiments of the present invention including, for example, methods for the in vitro binding of opioid receptors. In addition, if desired, such non-pharmaceutically acceptable salts may be converted to pharmaceutically acceptable salts by using techniques well known to the ordinarily skilled artisan, for example, by exchange of the acid that is non-pharmaceutically acceptable, for example, trifluoroacetic, perchloric or tetrafluoroboric acid, with an acid that is pharmaceutically acceptable, for example, the pharmaceutically acceptable acids described above.

Acid addition salts of the present invention include, for example, about one or more equivalents of monovalent acid per mole of the compound of the invention, depending in part on the nature of the acid as well as the number of basic lone pairs of electrons available for protonation. Similarly, acid addition salts of the present invention include, for example, about one-half or more equivalents of a divalent acid (such as, for example, sulfuric acid, oxalic acid or succinic acid) or about one third or more equivalents of trivalent acid (such as, for example, citric acid) per mole of the compound of the invention, depending in part on the nature of the acid as well as the number of basic lone pairs of electrons available for protonation. Generally speaking, the number of acid equivalents may vary up to about the number of equivalents of basic lone pairs of electrons in the compounds described herein.

Salts of the present invention which are derived from metal bases or basic amines include, for example, about one or more equivalents of monovalent metal or amine per mole of the compound of the invention, depending in part on the nature of the base as well as the number of available acidic protons. Similarly, salts of the present invention include, for example, about one-half or more equivalents of a divalent base (such as, for example, magnesium hydroxide or calcium hydroxide). Generally speaking, the number of basic equivalents may vary up to about the number of equivalents of acidic protons in the compounds described herein.

As used herein, the term “hydrate” refers to a compound or salt as described herein which is associated with water in the molecular form, i.e., in which the H—OH bond is not split, and may be represented, for example, by the formula R.H₂O, where R is a compound as described herein. A given compound or salt may form more than one hydrate including, for example, monohydrates (R.H₂O) or polyhydrates (R.nH₂O wherein n is an integer>1) including, for example, dihydrates (R.2H₂O), trihydrates (R.3H₂O), and the like, or hemihydrates, such as, for example, R.n_{/2}H₂O, R.n_{/3}H₂O, R.n_{/4}H₂O and the like wherein n is an integer.

As used herein, the term “solvate” refers to a compound or salt as described herein which is associated with solvent in the molecular form, i.e., in which the solvent is coordinatively bound, and may be represented, for example, by the formula R.(solvent), where R is a compound as described herein. A given compound or salt may form more than one solvate including, for example, monosolvates (R.(solvent)) or polysolvates (R.n(solvent)) wherein n is an integer>1) including, for example, disolvates (R.2(solvent)), trisolvates (R.3(solvent)), and the like, or hemisolvates, such as, for example, R.n_{/2}(solvent), R.n_{/3}(solvent), R.n_{/4}(solvent) and the like wherein n is an integer. Solvents herein include mixed solvents, for example, methanol/water, and as such, the solvates may incorporate one or more solvents within the solvate.

As used herein, the term “acid salt hydrate” refers to a complex that may be formed through association of a compound having one or more base moieties with at least one compound having one or more acid moieties, the complex being further associated with water so as to form a hydrate.

“Patient” refers to animals, including mammals, preferably humans.

When any variable occurs more than one time in any constituent or in any formula, its definition in each occurrence is independent of its definition at every other occurrence. Combinations of substituents and/or variables are permissible only if such combinations result in stable compounds.

When any variable occurs more than one time in any two or more diastereomers of a mixture, or in any two or more reactants of a process, its definition in each occurrence is independent of its definition at every other occurrence. Thus, for example, if one constituent of a mixture of diastereomers is shown to bear an R group, where R¹ is, for example, methyl, then other constituents of said mixture of diastereomers may each bear, independently, R¹ groups that are the same as, or different from, the R¹ of the first constituent, so long as they are selected from the defined list of R¹. Further, for example, if one reactant of a process is shown to bear an R¹ group, where R¹ is, for example, methyl, then other reactants of said process may each bear, independently, R¹ groups that are the same as, or different from, the R¹ of the first reactant, so long as they are selected from the defined list of R¹. Combinations of substituents and/or variables are permissible only if such combinations result in stable compounds.

The processes and synthetic methods described hereinthroughout may be carried out in any suitable solvent. Generally, suitable solvents are solvents which are substantially non-reactive with the starting materials (reactants), the intermediates, or products at the temperatures at which the reactions are carried out, i.e., temperatures which may range from the solvent's freezing temperature to the solvent's boiling temperature. A given reaction may be carried out in one solvent or a mixture of more than one solvent. Depending on the particular reaction, suitable solvents for a particular work-up following the reaction may be selected.

In certain embodiments, the diastereoselectivity of a reaction may be controlled or affected by the type of solvent employed, such as a protic solvent or an aprotic solvent. Protic solvents include, for example, water and alcohols such as methanol, ethanol, propanols, including n-propanol and isopropanol, butanols, including 1-butanol, 2-butanol, i-butanol, and t-butanol, substituted ethanols, including 2-nitroethanol, 2-fluoroethanol, 2,2,2-trifluoroethanol, 2-methoxyethanol and 2-ethoxyethanol, polyols, including ethylene glycol and diethylene glycol, pentanols, including 1-, 2-, or 3-pentanol, neo-pentanol, and t-pentanol, ethers, including monomethyl ether and diethylene glycol monoethyl ether, cyclic alcohols, including cyclohexanol, aromatic alcohols, including benzyl alcohol and phenol, and glycerol, to name a few.

Aprotic solvents include, for example, hydrocarbon solvents, and halogenated derivatives thereof, such as cyclohexane, pentane, toluene, benzene, cycloheptane, methylcyclohexane, ethylbenzene, m-, o-, or p-xylene, octane, indane, nonane, and the like. Aprotic solvents further include ethers, such as diethyl ether, dimethoxymethane, tetrahydrofuran (THF), alkylated tetrahydrofurans, including for example, methyltetrahydrofuran, preferably 2-methyltetrahydrofuran, 1,3-dioxane, 1,4-dioxane, furan, ethylene glycol dimethyl ether, ethylene glycol diethyl ether, diethylene glycol dimethyl ether, diethylene glycol diethyl ether, triethylene glycol diisopropyl ether, anisole, or t-butylmethyl ether. Other aprotic solvents include, for example, dimethylformamide (DMF), dimethylacetamide (DMAC), 1,3-dimethyl-3,4,5,6-tetrahydro-2(1H)-pyrimidinone (DMPU), 1,3-dimethyl-2-imidazolidinone (DMI), N-methyl-pyrrolidinone (NMP), formamide, N-methylacetamide, N-methylformamide, acetonitrile (MeCN), dimethylsulfoxide (DMSO), propionitrile, ethyl formate, methyl acetate, hexachloroacetone, acetone, ethyl methyl ketone, ethyl acetate, isopropyl acetate, t-butyl acetate, sulfolane, N,N-dimethylpropionamide, nitromethane, nitrobenzene, and hexamethylphosphoramide.

In accordance with certain embodiments of the present invention, processes described herein may be carried out such that contacting of compounds and reagents occurs in the presence of microwave energy. Microwave technology may help increase reaction rates of various addition reactions such as, for example, Michael addition and related reactions. The use of microwaves in accelerating reaction rates is well known in the art of synthetic organic chemistry, and is described, for example, in Lidstrom, et al. Tetrahedron, 2001, 57(45), 9225-9283, the disclosure of which is hereby incorporated herein by reference in its entirety.

Certain processes of the present invention may yield mixtures of diastereomers. Thus, in some embodiments, processes may, if desired, include a separation step to isolate diastereomers. Methods for separation of diastereomers are well known in the art and include, for example, chiral column chromatography, HPLC, recrystallization, or classical resolution methods involving selective reactivity.

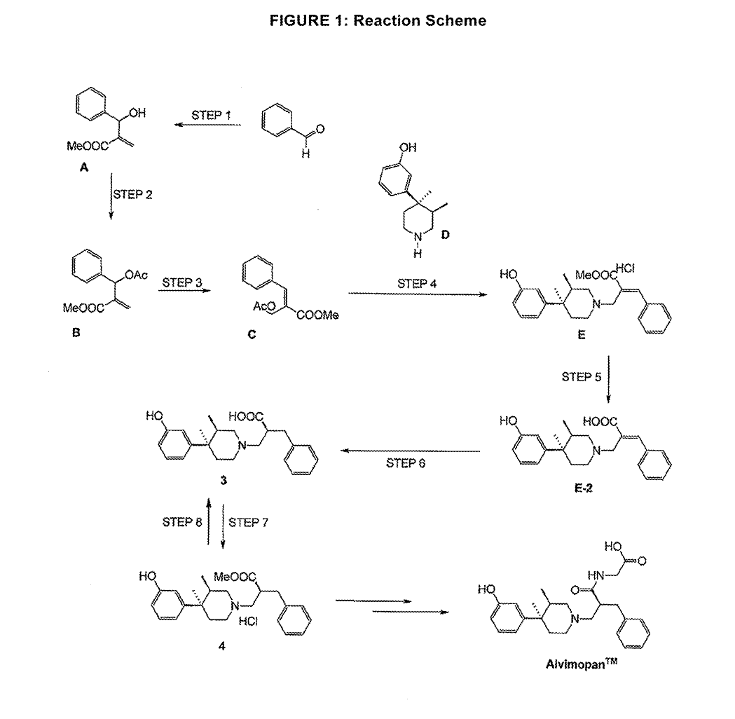

In preferred form, the processes and intermediates of the present invention provide for improved syntheses of Alvimopan and related compounds, such as intermediates, diastereomers, and salts of Alvimopan. More particularly, the present methods may, for example, desirably eliminate or replace the inefficient step(s) of transforming certain intermediates in prior art processes, advantageously resulting in higher overall yields, improved diastereoselectivity, and the like. Accordingly, the present methods and intermediates generally pertain to the modification of piperidine intermediates (see, e.g., intermediate D in FIG. 1). For example, catalytic hydrogenation of N-alkenyl piperidine intermediates to N-alkylpiperidine intermediates may proceed with high diastereoselectivity depending on the solvent in which the reaction is carried out.

Accordingly, in one embodiment, the present invention provides processes for preparing compounds of Formula Ia compounds of Formula Ib, and mixtures thereof:

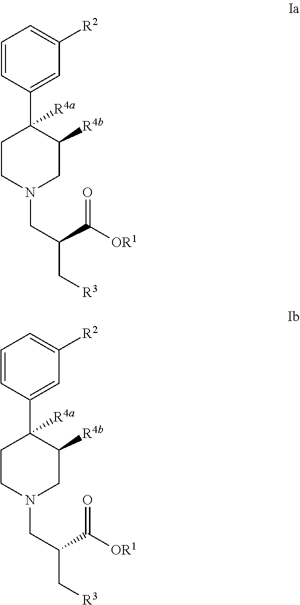

wherein:
each R¹ is independently H, alkyl, or aralkyl;
each R² is independently Cl, Br, I, —C(═O)OR^{5b}, —CN, —OR⁶, or —CONR⁷R⁸;
each R³ is independently H, alkyl, cycloalkyl, or aryl;
each R^{4a} and R^{4b} is independently C₁₋₆alkyl;
each R^{5b} is independently H or alkyl;
each R⁶ is independently H, alkyl, cycloalkyl, alkylcycloalkyl, aralkyl, or an hydroxyl protecting group;
each R⁷ is independently H, alkyl, cycloalkyl, alkylcycloalkyl, or aralkyl; and
each R⁸ is independently H, alkyl, aralkyl, or aryl;
or a salt thereof;
   comprising contacting an N-alkenylpiperidine compound of Formula IIa, Formula IIb, Formula IIc or Formula IId, or mixture thereof:

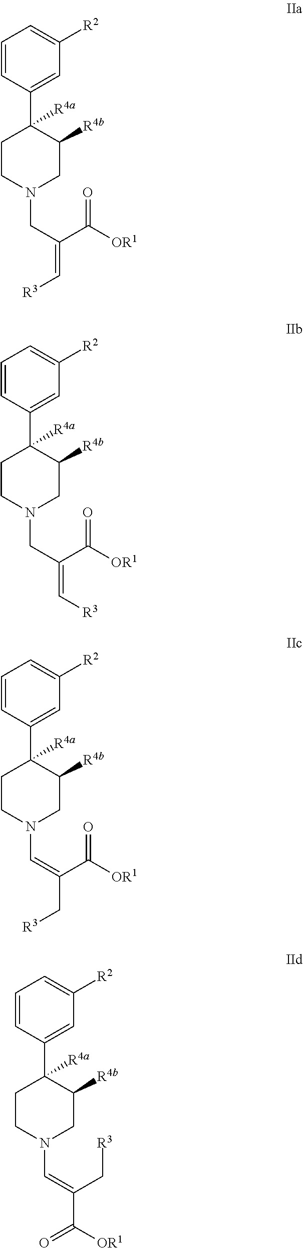

with hydrogen in the presence of a hydrogenation catalyst and a chiral phosphorus-containing ligand for a time and under conditions effective to provide the compound of Formula Ia, the compound of Formula Ib, or mixture thereof. A synthetic procedure that exemplifies the above preparatory process is depicted in FIG. 1.

In any of the hereinabove described processes and/or compounds of the present invention, each R¹ is independently H, alkyl, or aralkyl. In certain preferred embodiments, each R¹ is independently H or alkyl, more preferably H or C₁₋₆alkyl, still more preferably H or CH₃, with H being even more preferred. In some preferred embodiments wherein R¹ is C₁₋₆alkyl, it is more preferably methyl or ethyl.

Also any of the hereinabove described processes and/or compounds of the present invention, each R² is independently Cl, Br, I, —C(═O)OR^{5b}, —CN, —OR⁶, or —CONR⁷R⁸. In certain preferred embodiments, each R² is independently —C(═O)OR^{5b}, —CN, —OR⁶, or —CONR⁷R⁸, more preferably —OR⁶ or —CONR⁷R⁸, with —OR⁶ being even more preferred. In certain alternatively preferred embodiments, R² is —CONR⁷R⁸.

In any of the hereinabove described processes and/or compounds of the present invention, each R³ is independently H, alkyl, cycloalkyl, or aryl. In certain preferred embodiments, each R³ is independently alkyl, cycloalkyl or aryl, more preferably cycloalkyl or aryl, with aryl being even more preferred. In certain alternatively preferred embodiments, R³ is cycloalkyl. In embodiments wherein R³ is independently cycloalkyl, the cycloalkyl is preferably C₃₋₈ cycloalkyl, more preferably C₃₋₆ cycloalkyl, still more preferably C₆ cycloalkyl, with optionally substituted cyclohexyl being even more preferred. In embodiments wherein R³ is independently aryl, the aryl is preferably C₆₋₁₀ aryl, more preferably C₆ aryl, with optionally substituted phenyl being even more preferred.

Also any of the hereinabove described processes and/or compounds of the present invention, each R^{4a} and R^{4b} is independently C₁₋₆alkyl, preferably C₁₋₃alkyl, more preferably C₁₋₃alkyl, still more preferably C₁alkyl, with methyl being even more preferred.

In any of the hereinabove described processes and/or compounds of the present invention, each R^{5b} is independently H or alkyl. In embodiments wherein R^{5b} is alkyl, it is preferably C₁₋₁₀, more preferably C₁₋₆, still more preferably C₁₋₄, yet more preferably C₁₋₃alkyl, even more preferably C₁alkyl.

Also in any of the hereinabove described processes, each R⁶ is independently H, alkyl, cycloalkyl, alkylcycloalkyl, aralkyl, or an hydroxyl protecting group; preferably H, alkyl, aralkyl, or an hydroxyl protecting group; more preferably H, aralkyl, or an hydroxyl protecting group; still more preferably H or an hydroxyl protecting group, with H being even more preferred.

In any of the hereinabove described processes and/or compounds of the present invention, each R⁷ is independently H, alkyl, cycloalkyl, alkylcycloalkyl, or aralkyl. Also the above process, each R⁸ is independently H, alkyl, aralkyl, or aryl. In certain preferred embodiments, at least one of R⁷ and R⁸ is H; more preferably wherein R⁷ and R⁸ are H.

In certain preferred embodiments of any of the hereinabove described processes and/or compounds of the present invention, R¹ is independently H or C₁₋₆alkyl, R² is —OH, R³ is phenyl, and R^{4a} and R^{4b} are methyl.

In accordance with some preferred embodiments of the hereinabove described processes, the N-alkenylpiperidine compound of Formula IIa, Formula IIb, Formula IIc, or Formula IId, or mixture thereof is an N-alkenylpiperidine compound of Formula IIa or Formula IIb, or mixture thereof; more preferably an N-alkenylpiperidine compound of Formula IIa. Alternatively preferred, the N-alkenylpiperidine compound is an N-alkenylpiperidine compound of Formula IIb, while in still other alternatively preferred embodiments, the N-alkenylpiperidine compound comprises a mixture of N-alkenylpiperidine compounds of Formula IIa and Formula IIb.

In accordance with some other preferred embodiments, more preferably those embodiments wherein the N-alkenylpiperidine compound is an N-alkenylpiperidine compound of Formula IIa or Formula IIb, or mixture thereof, the N-alkylpiperidine compound prepared by hydrogenation of the compound of Formula IIa, IIb, IIc, or IId, or mixture thereof, preferably Formula IIa or IIb, or mixture thereof, forms a diastereomeric mixture of a compound of Formula Ia and a compound of Formula Ib, said diastereomeric mixture prepared with a molar ratio of greater than about 1 to about 1 based on the moles of the compound of Formula Ia to the moles of the compound of Formula Ib; more preferably with a molar ratio of at least about 2 to about 1; still more preferably at least about 4 to about 1; yet more preferably at least about 9 to about 1; with a molar ratio of at least about 19 to about 1 being even more preferred.

As noted hereinabove, and in accordance with some preferred embodiments, the N-alkylpiperidine compound prepared by hydrogenation is provided as a mixture of diastereomers. In some embodiments, the mixture is characterized by a diastereomeric excess of one compound relative to another. For example, mixtures provided in accordance with the present invention may have a diastereomeric excess of the compound of Formula Ia relative to the compound of Formula Ib or, conversely, a diastereomeric excess of the compound of Formula Ib relative to the compound of Formula Ia. In certain preferred, embodiments, the compound of Formula Ia is prepared in a diastereomeric excess of greater than about 1 relative to the compound of Formula Ib. More preferably, the compound of Formula Ia is prepared in a diastereomeric excess ranging from about 2:1 to about 100:1 (and all combinations and subcombinations of ranges and specific ratios therein), with from about 2:1 to about 10:1 being even more preferred relative to the compound of Formula Ib. In certain other preferred embodiments, the compound of Formula Ib is prepared in a diastereomeric excess of greater than about 1 relative to the compound of Formula Ia. More preferably, the compound of Formula Ib is prepared in a diastereomeric excess ranging from about 2:1 to about 100:1 (and all combinations and subcombinations of ranges and specific ratios therein) relative to the compound of Formula Ia, with from about 2:1 to about 10:1 being even more preferred. Methods for determining diastereomeric excess are well known to those skilled in the art and would be readily apparent once placed in possession of the present disclosure.

In accordance with certain preferred embodiments described herein, the N-alkenylpiperidine compound of Formula IIa or Formula IIb or mixture thereof is prepared by a process comprising contacting a piperidine compound of Formula III:

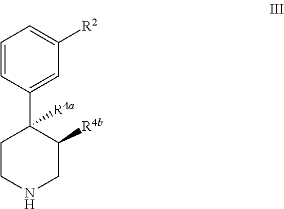

with an alkene compound of Formula IVa or Formula IVb or mixture thereof:

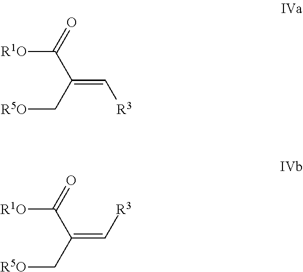

wherein:
each R¹, R², R³, R^{4a}, and R^{4b}, is independently as described hereinabove;
each R⁵ is independently alkyl, aralkyl, or —C(═O)R^{5a}; and
each R^{5a} is independently H, alkyl, or aralkyl;
   for a time and under conditions effective to provide the N-alkenylpiperidine compound of Formula IIa or Formula IIb, or mixture thereof.

In certain alternatively preferred processes for preparing N-alkenylpiperidine compounds of Formula IIa or Formula IIb, or mixture thereof, the moiety, R⁵—O— for compounds of formula IVa and/or IVb may be replaced by another satisfactory leaving group, such as for example, halide (e.g., chloride, bromide or iodide), or R⁵ may be an hydroxyl activating group, or any of the numerous leaving groups available to the synthetic organic chemist for use in displacement reactions.

In some preferred embodiments, each R⁵ is independently alkyl, aralkyl, or —C(═O)R^{5a}; with R⁵ is —C(═O)R^{5a} being even more preferred.

In other preferred embodiments, each R^{5a} is independently H, alkyl, or aralkyl; more preferably alkyl.

In accordance with some embodiments, the contacting of the N-alkenylpiperidine compound of Formula IIa, Formula IIb, Formula IIc or Formula IId, or mixture thereof may be carried out at any temperature that is effective to provide the N-alkenylpiperidine compound of Formula IIa or Formula IIb, or mixture thereof. In certain preferred embodiments, the contacting is carried out at a temperature of from about 10° C. to about 100° C.; more preferably from about 20° C. to about 85° C.; with a temperature of from about 20° C. to about 65° C. being even more preferred.

In accordance with other embodiments, the contacting of the N-alkenylpiperidine compound of Formula IIa, Formula IIb, Formula IIc or Formula IId, or mixture thereof may be carried out at any hydrogen charging pressure that is effective to provide the N-alkylpiperidine compound of Formula Ia or Formula Ib, or mixture thereof. Preferably, in some embodiments, the contacting is carried out in a reactor into which hydrogen gas is charged at a pressure of from about 1 bar to about 150 bar; more preferably from about 1 bar to about 80 bar; still more preferably from about 3 bar to about 50 bar; yet more preferably from about 3 bar to about 30 bar, still more preferably from about 3 to about 15 bar.

While a wide range of catalyst loadings may be employed in the contactings of the hereinabove disclosed hydrogenation processes, it may be useful, in certain embodiments, to set the catalyst level based on a molar ratio of the catalyst to the N-alkenylpiperidine compound of Formula IIa, Formula IIb, Formula IIc or Formula IId, or mixture thereof. Accordingly, in certain preferred processes, the molar ratio of the N-alkenylpiperidine compound to the hydrogenation catalyst is from about 10 to about 50,000; more preferably from about 100 to about 10,000, still more preferably from about 100 to about 4,000, yet more preferably from about 100 to about 3000.

The time of contacting in the above described hydrogenation processes is generally not critical. By way of general guidance, the contacting may be carried out for from about 10 minutes to about 250 hours, preferably from about 1 to about 100 hours; more preferably from about 1 to about 24 hours; with from about 2 to about 20 hours being even more preferred.

The hydrogenation catalyst or catalysts employed in the contactings of the above disclosed processes may be heterogeneous or homogeneous. In some preferred embodiments the catalysts are heterogeneous. In other preferred embodiments, the catalysts are homogenous. In embodiments wherein the catalyst is heterogeneous, it is preferably a catalyst comprising palladium. In embodiments wherein the catalyst is homogeneous, it is preferably a catalyst comprising a Group VIII transition metal, preferably wherein the Group VIII transition metal catalyst comprises rhodium, ruthenium, or iridium, more preferably rhodium.

In addition to the hydrogenation catalyst, the processes comprising a contacting with hydrogen may also employ a phosphorus-containing ligand. In some preferred embodiments, the ligand is chiral, more preferably, the phosphorus-containing ligand is a chiral tertiary diphosphine. Exemplary ligands include:

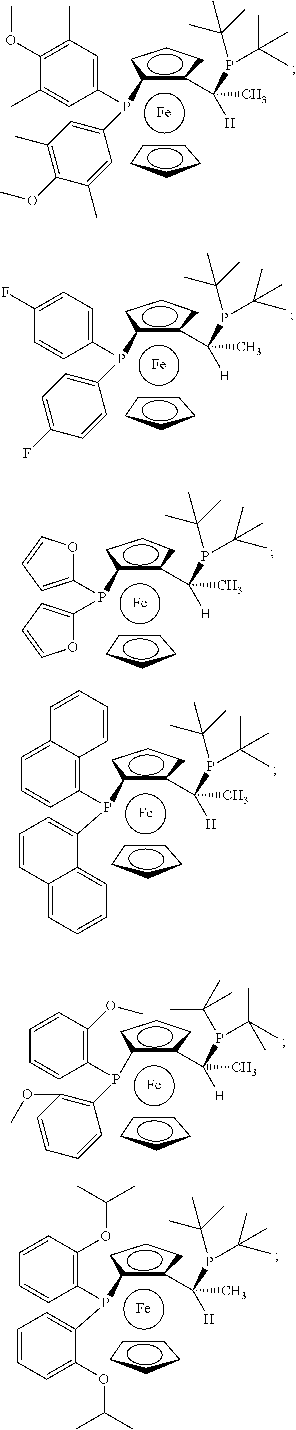

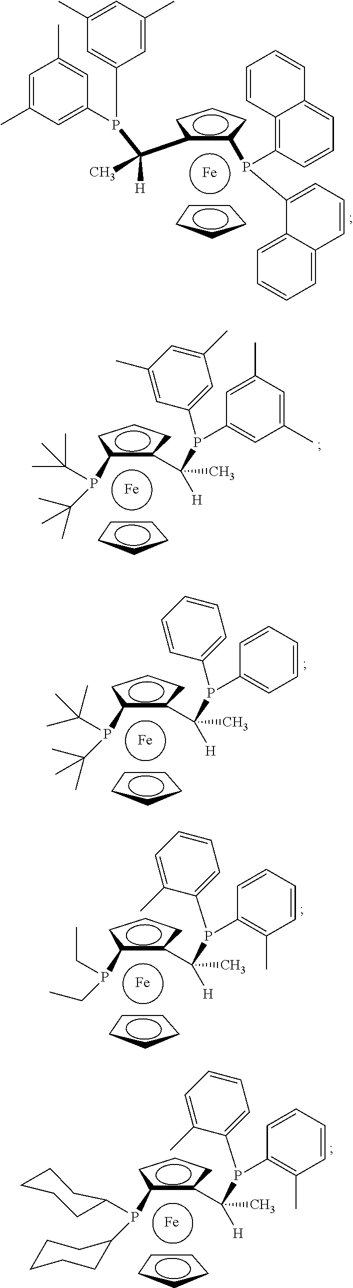

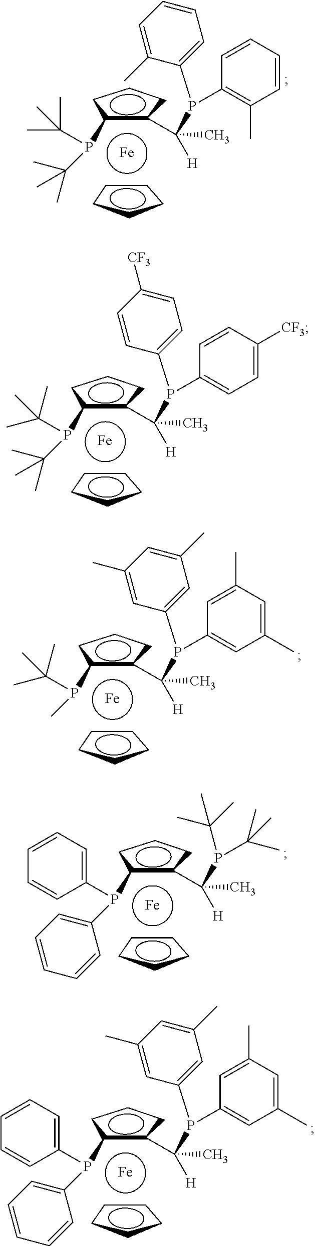

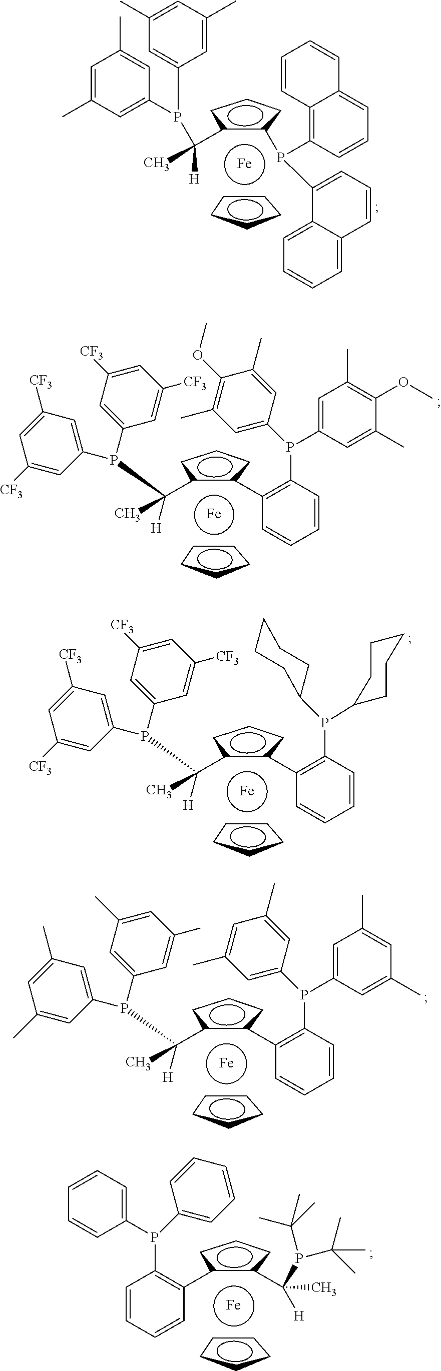

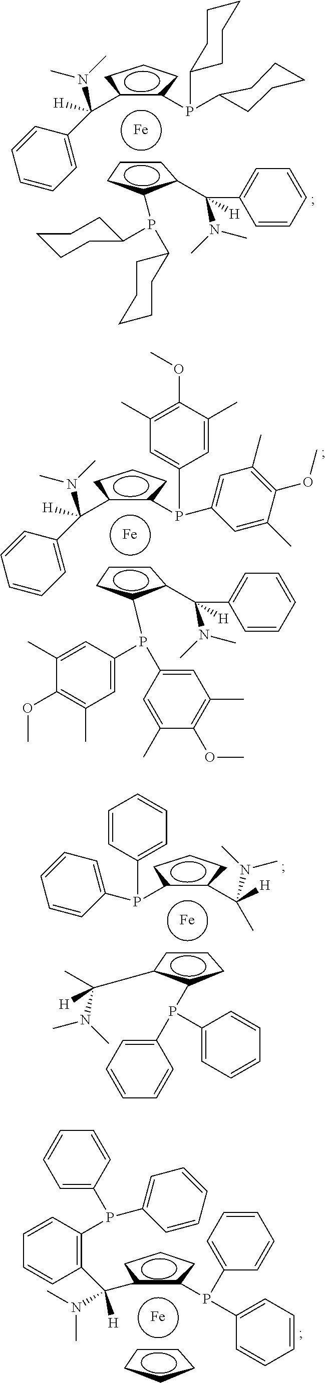

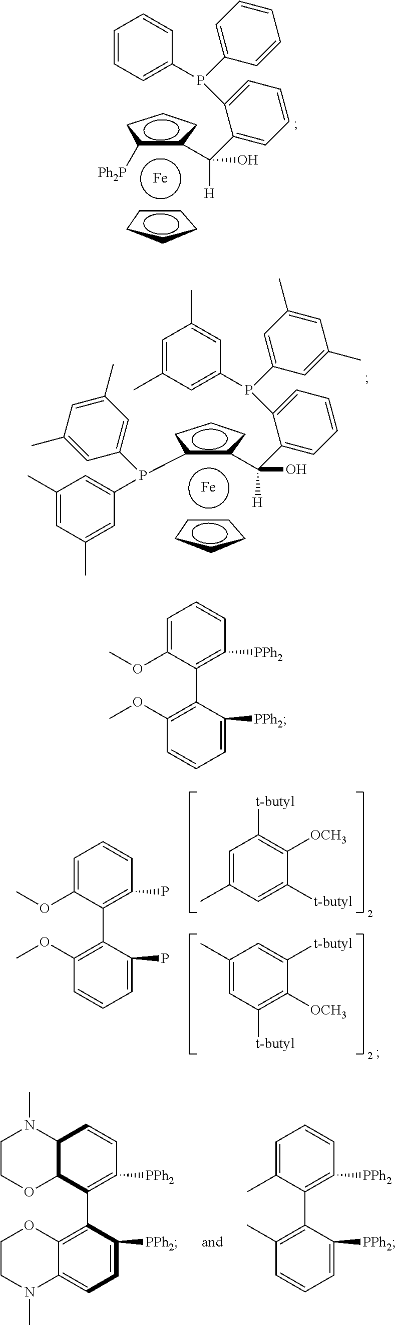

or a mixture thereof. In other embodiments, the enantiomer of the phosphorus-containing ligands drawn above is employed in the hydrogenation reaction.

In certain preferred embodiments, the chiral tertiary diphosphine is selected from the group consisting of:

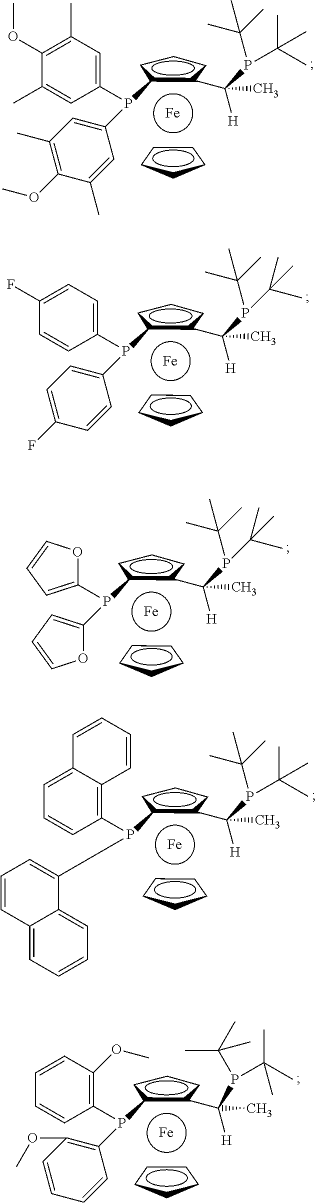

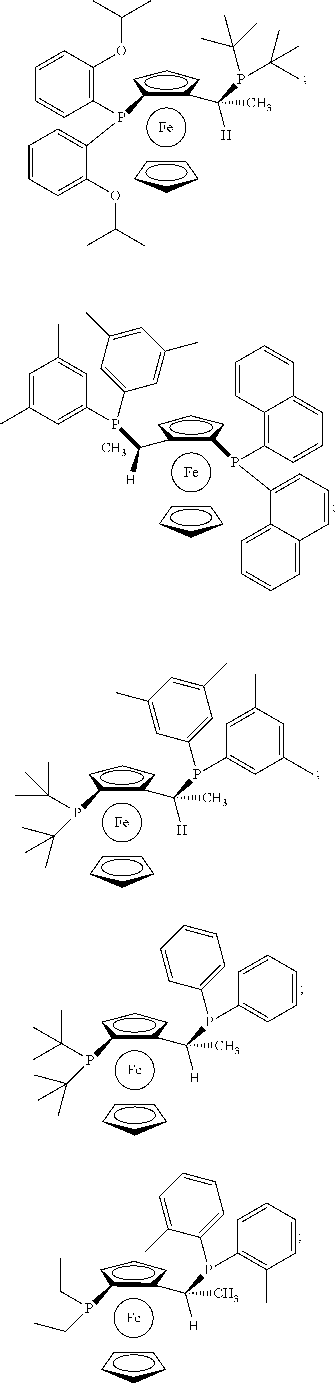

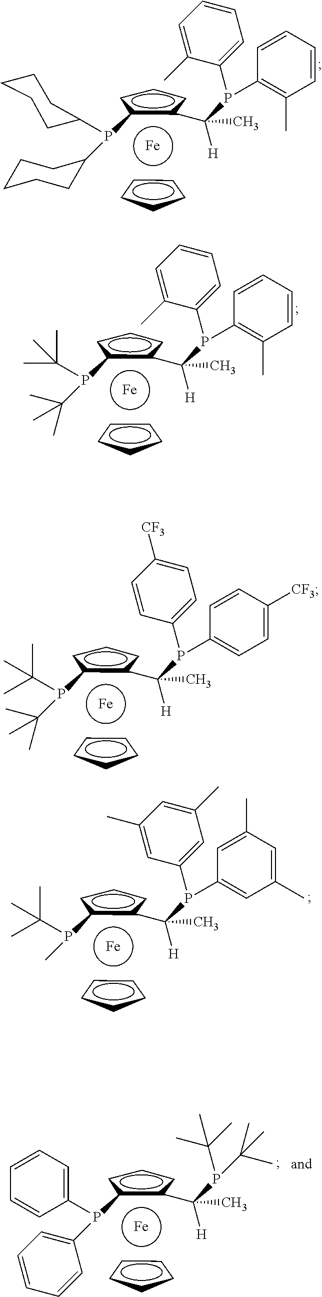

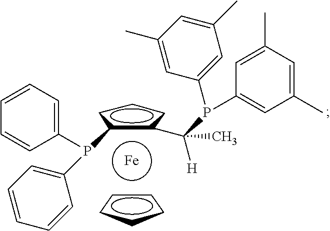

or a mixture thereof. In other embodiments, the enantiomer of the phosphorus-containing ligands drawn above is employed in the hydrogenation reaction.

Alternatively preferred in certain embodiments, the chiral tertiary diphosphine is selected from the group consisting of:

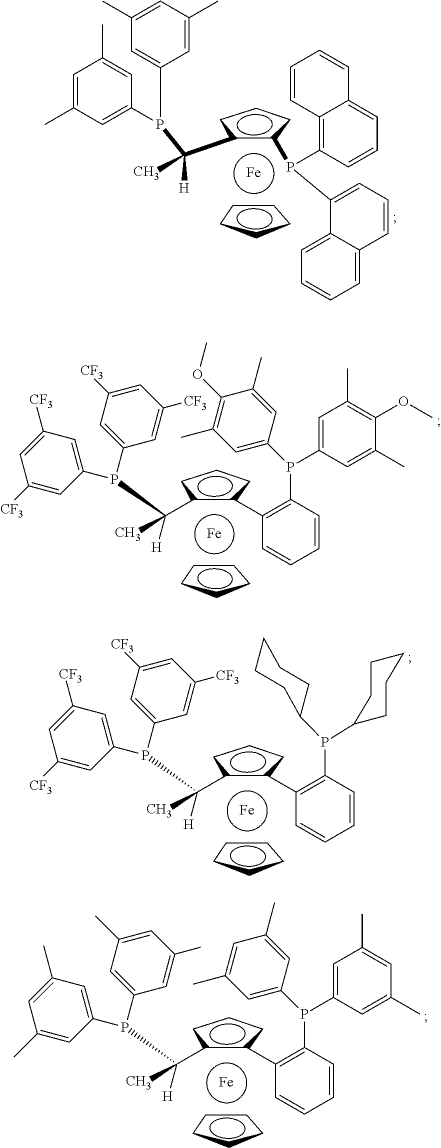

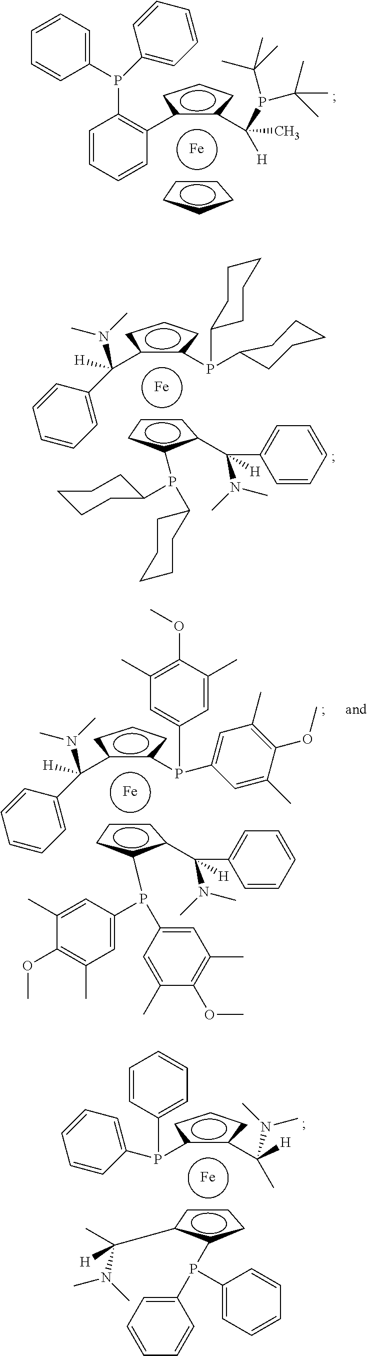

or a mixture thereof. In other embodiments, the enantiomer of the phosphorus-containing ligands drawn above is employed in the hydrogenation reaction.

In still other alternatively preferred embodiments, the chiral tertiary diphosphine is selected from the group consisting of:

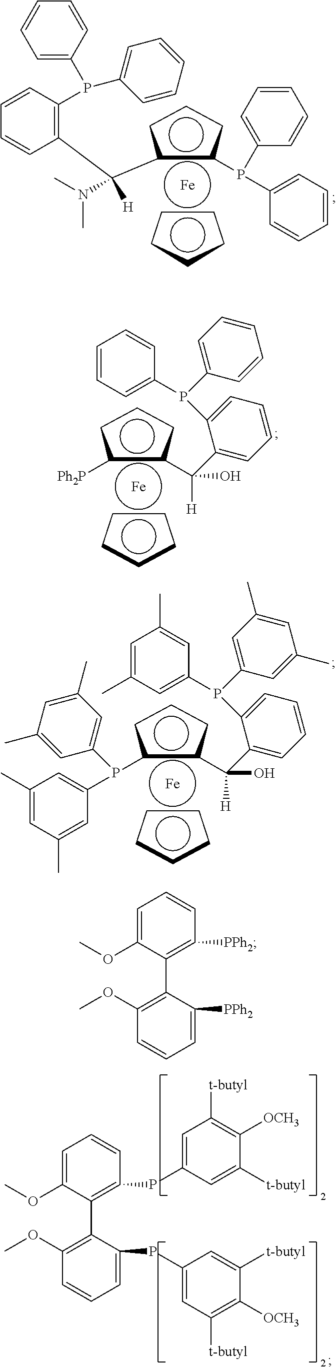

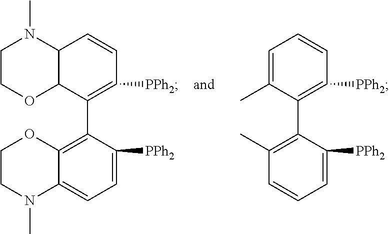

or a mixture thereof. In other embodiments, the enantiomer of the phosphorus-containing ligands drawn above is employed in the hydrogenation reaction.

Other exemplary chiral tertiary diphosphine ligands that may be employed in some preferred embodiments of the present invention include:

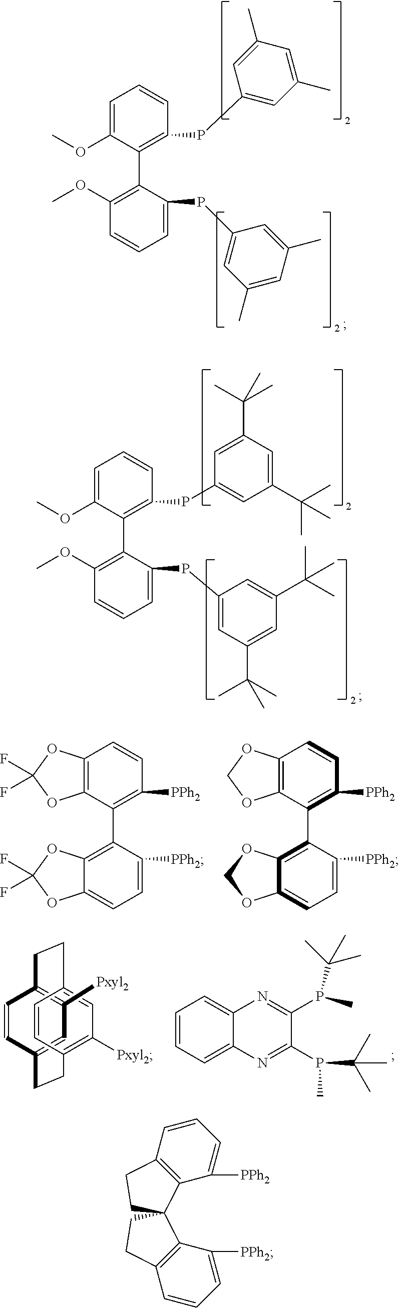

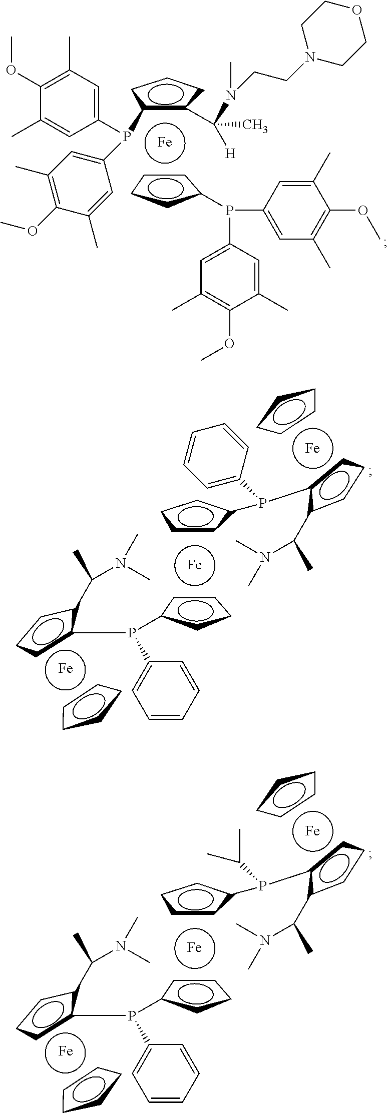

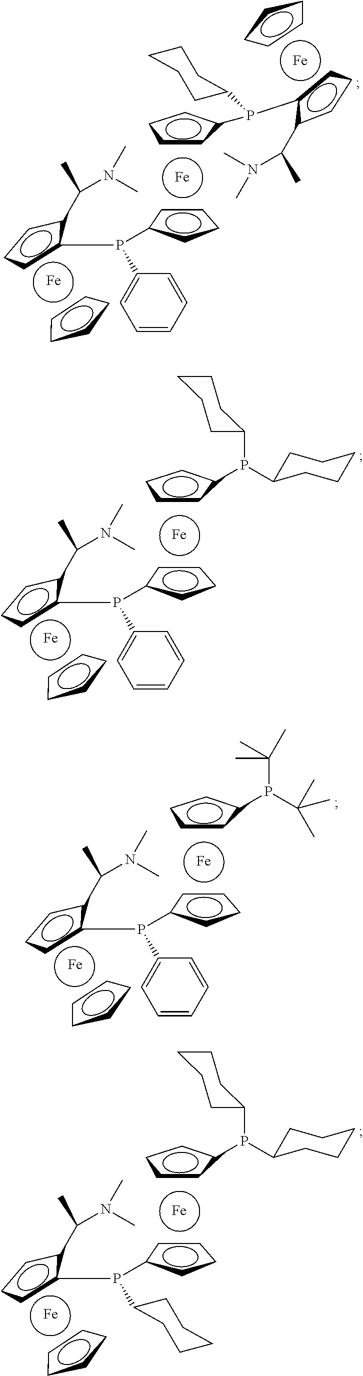

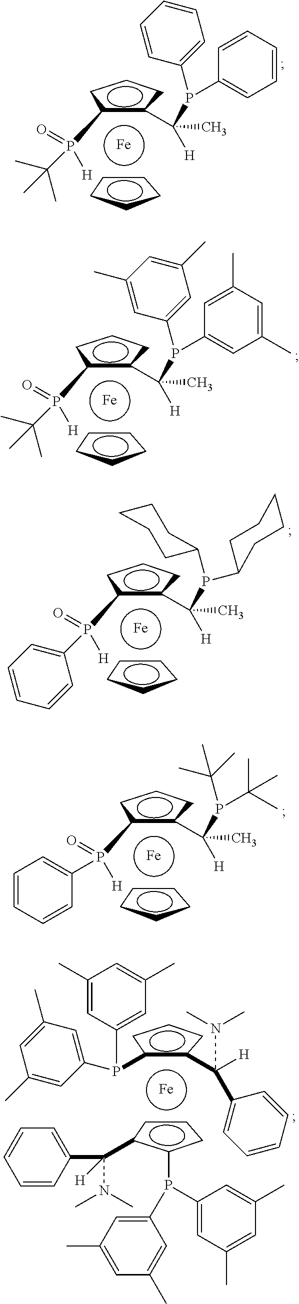

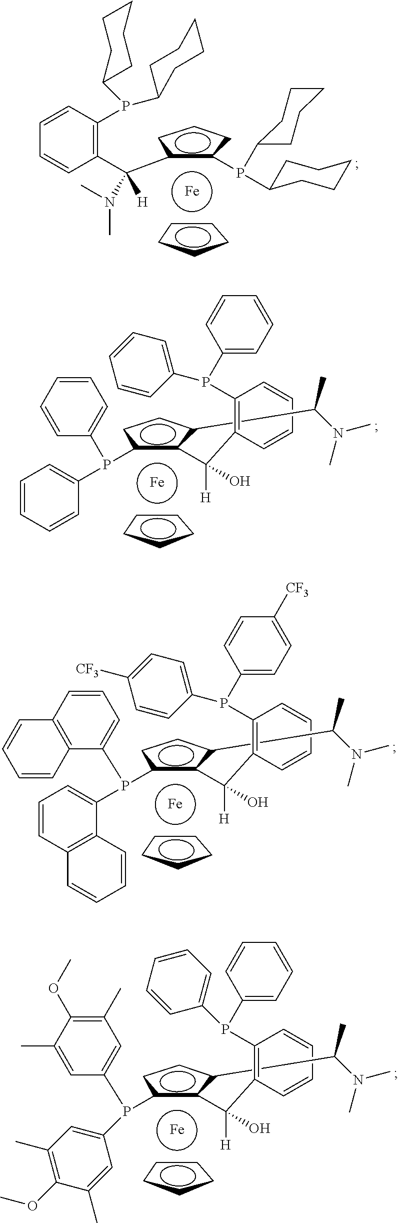

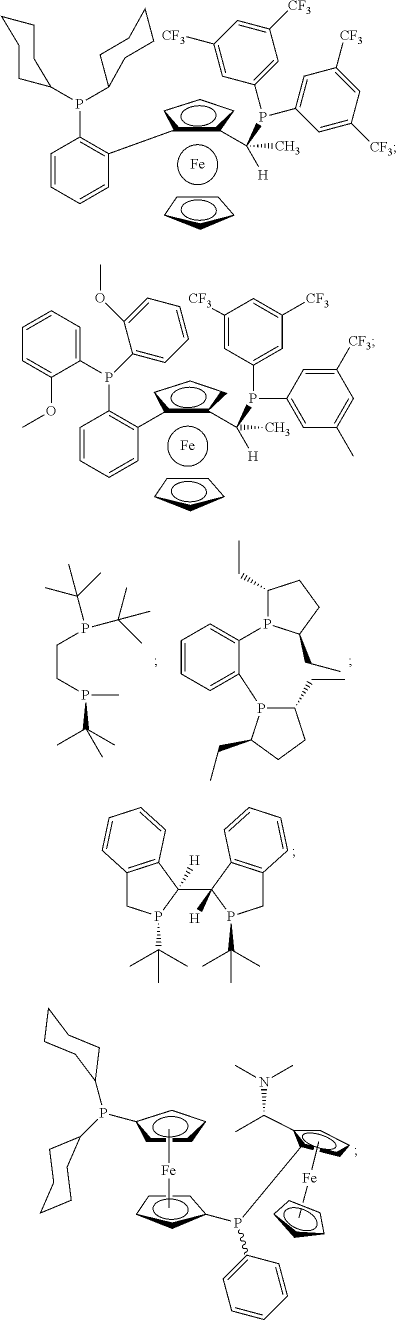

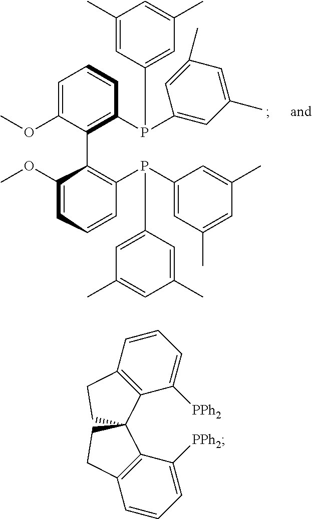

or a mixture thereof. In other embodiments, the enantiomer of the phosphorus-containing ligands drawn above is employed in the hydrogenation reaction.

In still other preferred embodiments of the present invention; the chiral tertiary diphosphine is selected from the group consisting of:

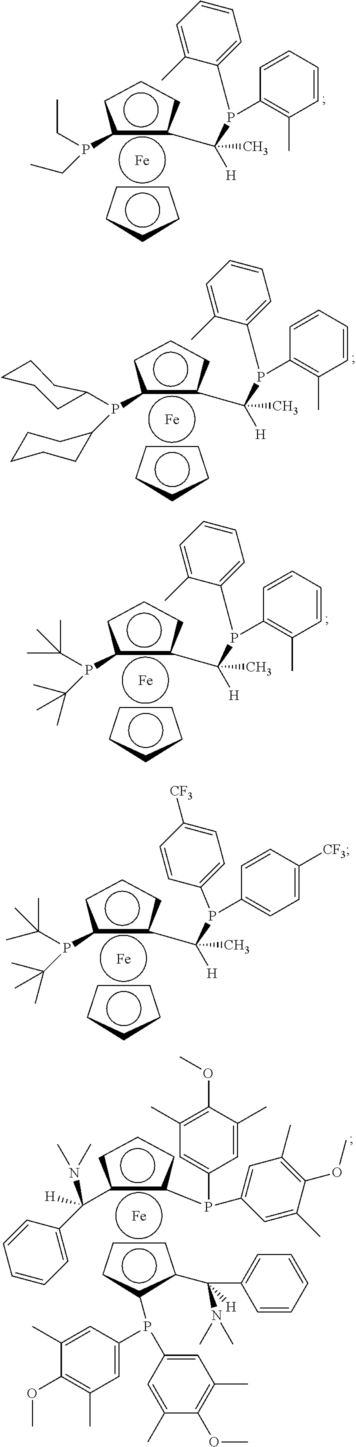

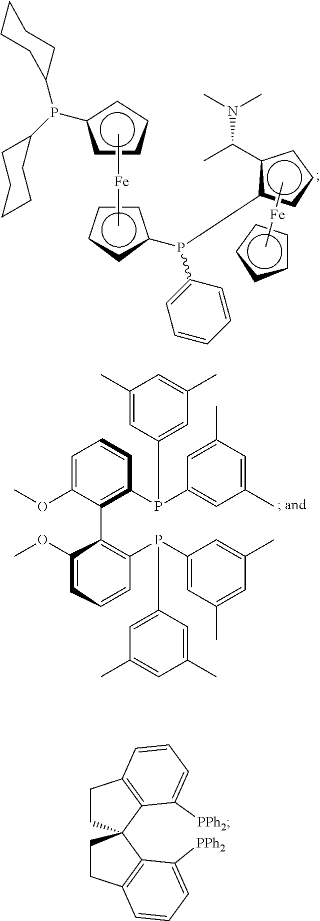

or a mixture thereof. In other embodiments, the enantiomer of the phosphorus-containing ligands drawn above is employed in the hydrogenation reaction.

In accordance with some preferred embodiments, the contacting of the N-alkenylpiperidine compound of Formula IIa, Formula IIb, Formula IIc or Formula IId, or mixture thereof with hydrogen may be carried out in solution comprising a protic or aprotic solvent. Exemplary solvents include alcoholic solvents, ethers, aromatic hydrocarbons, chlorinated hydrocarbons, esters or lactones, or mixture thereof. The particular type of solvent chosen may affect the diastereoselectivity of the reaction.

According to certain other preferred embodiments, the contacting with hydrogen is carried out in a protic solvent, such as an alcohol, optionally further comprising water. Suitable alcohols may have the formula R¹⁰OH, where R¹⁰ is an alkyl group as hereinbefore defined. Exemplary alcohols include methanol, ethanol, isopropanol, n-propanol, any of the isomeric butanols, and the like. More preferably, the alcohol solvent comprises an alcohol wherein the R¹⁰ alkyl group is C₁₋₆alkyl, still more preferably C₁₋₃alkyl. In certain even more preferred embodiments, the protic solvent comprises methanol, said methanol further optionally comprising water. In certain more preferred embodiments wherein the solvent comprises methanol and further comprises water, the amount of water present is from about 0.01% to about 1% by volume based on the volume of methanol solvent present in the reaction; more preferably from about 0.01% to about 0.75% by volume; with from about 0.01% to about 0.5% by volume being even more preferred.

In accordance with some embodiments, the contacting of the N-alkenylpiperidine compound of Formula IIa, Formula IIb, Formula IIc or Formula IId, or mixture thereof may be carried out with one or more additives to improve one or more aspects of the catalytic hydrogenation providing the N-alkylpiperidine compound of Formula Ia or Formula Ib, or mixture thereof. The additive comprises a proton acid additive or amine, preferably a proton acid additive. In some other preferred embodiments, the additive comprises an amine, still more preferably a difunctional amine, yet more preferably wherein the amine is N,N,N,N-tetramethylguanidine. In preferred embodiments wherein the additive comprises a proton acid, more preferably the proton acid is selected from the group consisting of an alkylsulfonic acid, an arylsulfonic acid, sulfuric acid, hydrochloric acid, and a carboxylic acid, still more preferably selected from the group consisting of an alkylsulfonic acid and an arylsulfonic acid, yet more preferably an alkylsulfonic acid, with methanesulfonic acid being even more preferred. In yet other preferred embodiments, wherein the additive comprises a proton acid, the proton acid is preferably sulfuric acid. In still other preferred embodiments, the proton acid is a carboxylic acid, more preferably trifluoroacetic acid.

In another embodiment, the present invention provides processes for preparing N-alkenylpiperidine compounds of Formula IIa or Formula IIb, or mixture thereof:

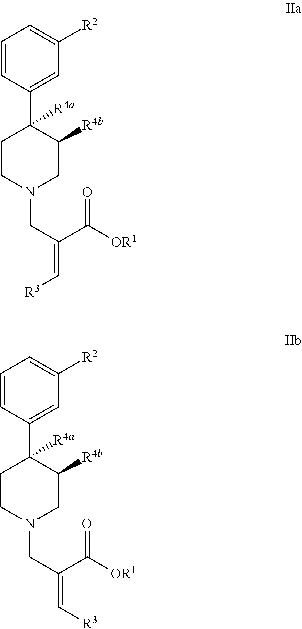

or a salt thereof;
   comprising contacting a piperidine compound of Formula III:

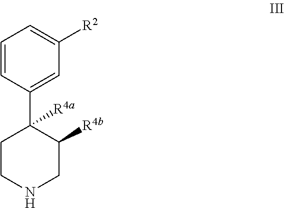

with an alkene compound of Formula IVa or Formula IVb, or mixture thereof:

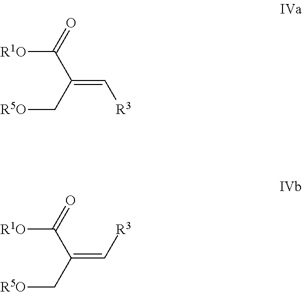

wherein:
   each R¹, R², R³, R^{4a}, R^{4b}, R⁵, R^{5b}, R⁶, R⁷, and R⁸ is independently as described hereinabove;
for a time and under conditions effective to provide the N-alkenylpiperidine compound of Formula IIa, Formula IIb, or mixture thereof. In certain alternatively preferred processes for preparing N-alkenylpiperidine compounds of Formula IIa or Formula II, or mixture thereof, the moiety, R⁵—O— for compounds of formula IVa and/or IVb may be replaced by another satisfactory leaving group, such as for example, halide (e.g., chloride, bromide or iodide), or R⁵ may be an hydroxyl activating group, or any of the numerous leaving groups available to the synthetic organic chemist in displacement reactions.

In certain preferred embodiments of the processes for preparing N-alkenylpiperidine compounds of Formula IIa or Formula IIb, or mixture thereof, the molar ratio of the compound of formula IIa to the compound of formula IIb is in the range of from about 5:1 to about 99.5:0.5; preferably from about 10:1 to about 99.5:0.5; still more preferably from about 19:1 to about 99.5:0.5; with from about 98:2 to about 99.5:0.5 being even more preferred.

A wide variety of hydroxyl activating groups are available and would be suitable for use in the present contacting of compounds of Formula III with an alkene of Formula IVa or IVb, or mixture thereof. In preferred embodiments, the hydroxyl activating group is, independently, alkylcarbonyl, arylcarbonyl, aralkylcarbonyl, heteroarylcarbonyl, heterocyclylcarbonyl, C(S)O-aryl, C(S)O-alkyl, or R^{Z}₃Si—, wherein each R^{Z} is, independently, alkyl or aryl, with alkylcarbonyl being more preferred. A particularly preferred hydroxyl activating agent is (—C(O)CH₃).

In accordance with some preferred embodiments, contacting the compound of Formula III with a compound of Formula IVa or Formula IVb, or mixture thereof may be carried out in solution comprising a protic or aprotic solvent. The particular type of solvent chosen may affect the regioselectivity of the reaction.

According to certain preferred embodiments, contacting is carried out in a protic solvent, such as an alcohol. Suitable alcohols may have the formula R¹⁰OH, where R¹⁰ is an alkyl group as hereinbefore defined. Exemplary alcohols include methanol, ethanol, isopropanol, n-propanol, any of the isomeric butanols, and the like. In preferred embodiments, the protic solvent is methanol. In other preferred embodiments, contacting is carried out in an aprotic solvent such as an ether. Any ether is suitable, including, for example non-cyclic ethers, and cyclic ethers, such as THF or dioxane. In certain more preferred embodiments, the solvent comprises THF. In other more preferred embodiments, the solvent comprises dioxane.

Thus, contacting the compound of Formula III with a compound of Formula IVa or Formula IVb, or mixture thereof in the hereinabove described processes may result in the preparation of a compound of Formula IIa, a compound of Formula IIb, a compound of Formula IIc, or a compound of Formula IId, or mixture thereof; preferably a compound of Formula IIa, a compound of Formula IIb, or mixture of compounds of Formulas IIa and IIb.

Contacting the compound of Formula III with a compound of Formula IVa or Formula IVb, or mixture thereof may be conducted under conditions, for example, temperature, and for a time effective to provide compounds of Formulas IIa, IIb, IIc, and/or IId, preferably IIa or IIb. By way of general guidance, the reaction may be conducted over a wide range of temperatures. Preferably, the reaction is conducted at a temperature and for a time sufficient to form compounds of Formulas IIa, IIb, IIc, and/or IId, preferably Formulas IIa and/or IIb. The particular temperatures and times may vary, depending, for example, on the particular Formula III and Formula IVa compounds involved, as well as the particular solvent employed. In preferred form, the reaction may be conducted at a temperature of from about −78° C. to about 150° C., with from about −20° C. to about 50° C. being more preferred. The reaction may be conducted for a suitable period of time, for example, from about 1 minute to about 7 days, preferably from about 30 minutes to about 48 hours, more preferably from about 1 hour to about 24 hours, still more preferably from about 2 to about 16 hours, with from about 4 to about 12 hours being even more preferred. The reaction may be monitored by any of a number of standard analytical techniques, such as thin layer chromatography (TLC).

In embodiments in which a mixture of compounds of Formula IIa and IIb, or a mixture of compounds of Formula Ia and Ib are prepared, the present processes may further include a step for separating the compounds of Formula IIa and Formula IIb, or the compounds of Formula Ia and Ib. For example, a diastereomeric mixture of compounds of Formulas Ia and Ib may be separated using any suitable method in the art. In some embodiments, separation may be carried out by chiral column chromatography, HPLC, recrystallization, or classical resolution methods. Other methods for separating the diastereomeric mixtures would be readily apparent to one ordinarily skilled in the art, once placed in possession of the present disclosure.

The compounds of Formula Ia and/or Formula Ib may undergo further transformations in accordance with the methods of the present invention. In this regard, the present invention provides processes for preparing N-alkylpiperidine compounds of Formula Va or Formula Vb, or mixture thereof:

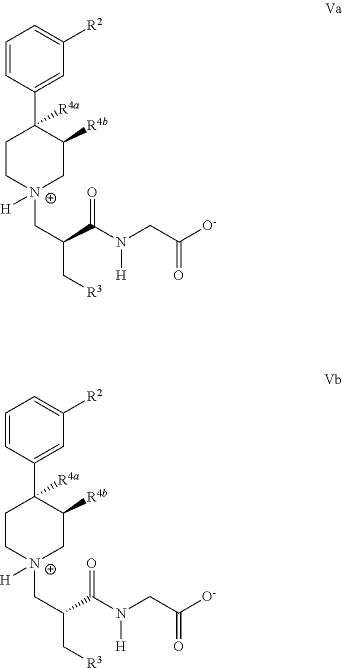

or a salt thereof;

comprising providing an N-alkenylpiperidine compound of Formula IIa, Formula IIb, Formula IIc, or Formula IId, or mixture thereof:

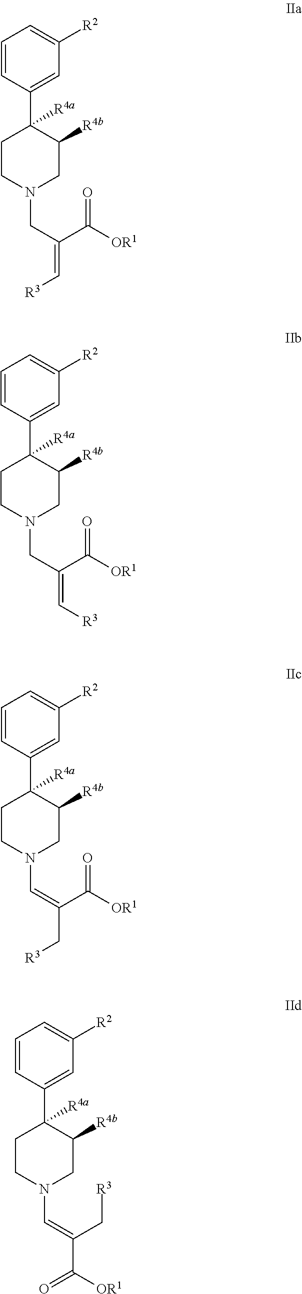

contacting the N-alkenylpiperidine compound of Formula IIa, Formula IIb, Formula IIc or Formula IId, or mixture thereof, with hydrogen in the presence of a hydrogenation catalyst and a chiral phosphorus-containing ligand for a time and under conditions effective to provide a compound of Formula Ia, a compound of Formula Ib, or mixture thereof:

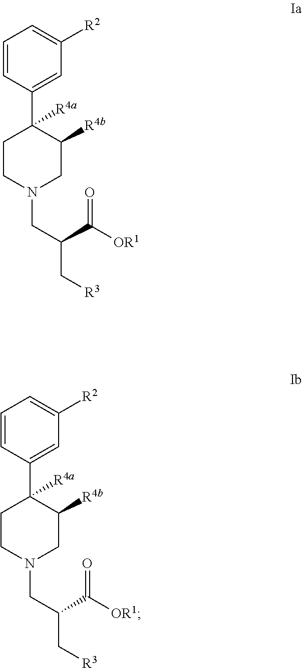

wherein:
each R¹, R², R³, R^{4a}, and R^{4b} is independently as described hereinabove; and

contacting the compound of Formula Ia, Formula Ib, or mixture thereof, with NH₂CH₂CO₂H for a time and under conditions effective to provide the compound of Formula Va, Formula Vb, or mixture thereof.

The processes comprise providing a compound of Formula Ia, a compound of Formula Ib, or mixture thereof (which may be prepared, for example, employing a method described herein), and selectively converting the —OR¹ moiety of the compound of Formula Ia, the compound of Formula Ib, or mixture thereof to —NHCH₂COOH. In accordance with certain embodiments, this selective conversion may proceed directly from compounds of Formula Ia and/or Ib. In accordance with other preferred embodiments, the conversion may first involve optionally converting —OR¹ of the compounds of Formula Ia and/or Ib to —X, where X is halo or —OC(O)R¹. Techniques for the optional conversion of the compounds of Formula Ia and/or Ib to acid halides or acid anhydrides are well known in the art, and are described, for example, in Larock, R. C., Comprehensive Organic Transformations, VCH Publishers, Inc., NY (1989), and Carey, F. A., and Sundberg, R. J., Advanced Organic Chemistry, 3^{rd} Edition, Plenum Press, NY (1990), the disclosures of each of which are hereby incorporated herein by reference in their entireties.

In the preparation of the compounds of Formulas Va and/or Vb from the compounds of Formulas Ia and/or Ib, each R¹ in compounds Ia and/or Ib is, independently, H, alkyl, or aralkyl. In certain preferred embodiments, In certain preferred embodiments, each R¹ is independently H or alkyl, more preferably H or C₁₋₆alkyl, still more preferably H or CH₃, with H being even more preferred. In some preferred embodiments wherein R¹ is C₁₋₆alkyl, it is more preferably methyl or ethyl. Also in the preparation of the compounds of Formulas Va and/or Vb from the compounds of Formulas Ia and/or Ib, each R² is independently Cl, Br, I, —C(═O)OR^{5b}, —CN, —OR⁶, or —CONR⁷R⁸. In certain preferred embodiments, each R² is independently —C(═O)OR^{5b}, —CN, —OR⁶, or —CONR⁷R⁸, more preferably —OR⁶ or —CONR⁷R⁸, with —OR⁶ being even more preferred. In certain alternatively preferred embodiments, R² is —CONR⁷R⁸. In even more preferred embodiments, the above transformation reaction provides Alvimopan and/or diastereomers thereof. In any of these preferred embodiments, each R^{5b}, R⁶, R⁷, and R⁸ is independently as described hereinabove.

A wide variety of techniques are available for selectively converting —OR¹ to —NHCH₂COOH in the above process, and would be readily apparent to one of ordinary skill in the art, once armed with the teachings of the present disclosure. Suitable conversion techniques are described, for example, in Werner et al., J. Org. Chem., 1996, 61, 587, the disclosure of which is hereby incorporated herein by reference in its entirety. An example of a suitable selective conversion includes contacting NH₂CH₂COOH, or an acid addition salt, ester or other derivative thereof with a compound of Formula Ia, Formula Ib, or mixture thereof.

Contacting a compound of Formula Ia and/or Ib with a compound NH₂CH₂COOH may be carried out in a protic solvent, such as an alcohol, or in an aprotic solvent such as an ether. Suitable alcohols and ethers include those discussed hereinthroughout. In addition, the conversion may be conducted under conditions, for example, temperature, and for a time effective to provide compounds of Formulas Va and/or Vb. The particular temperatures and times may vary, depending, for example, on the particular Formula Ia and/or Ib compounds involved, as well as the particular solvent employed. In preferred form, the reaction may be conducted at a temperature of from about −20° C. to about 100° C., with from about 0° C. to about 25° C. being more preferred. The reaction may be conducted for a suitable period of time, for example, from about 5 minutes to about 48 hours, preferably from about 1 hour to about 24 hours. The reaction may be monitored by standard analytical techniques, such as thin layer chromatography (TLC).

The invention is also directed, in part, to compounds of Formula IIa:

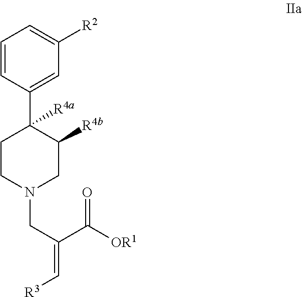

wherein:
R¹, R², R³, R^{4a}, and R^{4b} are as described hereinabove.

The invention is also directed, in part, to compounds of Formula IIb:

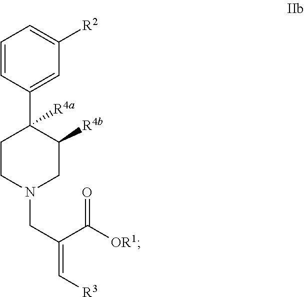

wherein:
   R¹, R², R³, R^{4a}, and R^{4b} are as described hereinabove.

The invention is also directed, in part, to compounds of Formula IIc:

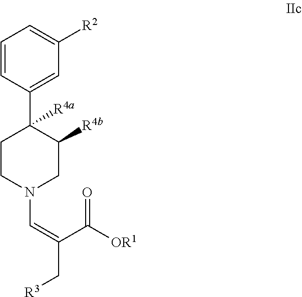

wherein:
   R¹, R², R³, R^{4a}, and R^{4b} are as described hereinabove.

The invention is also directed, in part, to compounds of Formula IId:

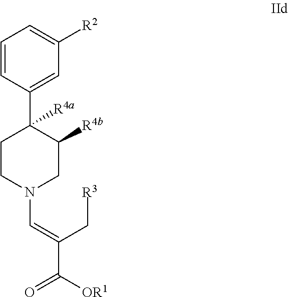

wherein:
R¹, R², R³, R^{4a}, and R^{4b} are as described hereinabove.

Compounds within the scope of the present invention including, for example, compounds of Formulas Ia, Ib, Va or Vb, may also exhibit significant activity as opioid antagonist compounds, including mu, kappa and delta opioid antagonist activity, and thereby may desirably possess therapeutic value, for example, in the treatment of gastro-intestinal motility disorders. In particular, compounds of the present invention may be useful in blocking peripheral opioid receptors, thereby providing utility for preventing and/or treating ileus. The term “ileus”, as used herein, refers to the obstruction of the bowel or gut, especially the colon. See, e.g., Dorland's Illustrated Medical Dictionary, p. 816, 27th ed. (W. B. Saunders Company, Philadelphia 1988). Ileus should be distinguished from constipation, which refers to infrequent or difficulty in evacuating the feces. See, e.g., Dorland's Illustrated Medical Dictionary, p. 375, 27th ed. (W. B. Saunders Company, Philadelphia 1988). Ileus may be diagnosed by the disruption of normal coordinated movements of the gut, resulting in failure of the propulsion of intestinal contents. See, e.g., Resnick, J. Am. J. of Gastroenterology 1997, 92, 751 and Resnick, J. Am. J. of Gastroenterology, 1997, 92, 934. In some instances, particularly following surgery, including surgery of the abdomen, the bowel dysfunction may become quite severe, lasting for more than a week and affecting more than one portion of the GI tract. This condition is often referred to as post-surgical (or post-operative) paralytic ileus and most frequently occurs after laparotomy (see Livingston, E. H. and Passaro, E. D. Jr. Digestive Diseases and Sciences 1990, 35, 121). “Post-surgical ileus”, which may follow surgery such as laparotomy, may be characterized by such symptoms as, for example, obstruction of the gut, particularly in the colon, resulting in nausea, vomiting, lack of passage of flatus and/or stools, abdominal distention and lack of bowel sounds. This condition generally lasts from about 3 to about 5 days, but may endure longer, including up to about one week. Longer durations are generally characteristic of a more severe form of ileus, termed post-surgical paralytic ileus, which may affect other portions of the GI tract in addition to the colon. Similarly, post-partum ileus is a common problem for women in the period following childbirth, and is thought to be caused by similar fluctuations in natural opioid levels as a result of birthing stress. “Post-partum ileus” generally refers to obstruction of the gut, particularly the colon, following parturition. Both natural and surgically-assisted procedures during parturition may lead to post-partum ileus treated by the present invention. Symptoms of postpartum ileus and post-surgical ileus are similar.

Compounds of the present invention may also be useful in preventing and/or treating peripheral opiate induced side effects. These side effects may be induced by administration of an opiate such as morphine to a mammal. The opiate induced side effects may include, for example, constipation, nausea, and vomiting. Thus, compounds of this invention may be useful for treating one or more opiate induced side effects. Compounds as described herein may also be useful in the treatment of irritable bowel syndrome, non-ulcer dyspepsia, and idiopathic constipation. Compounds of the invention do not substantially pass through the blood-brain barrier and therefore do not mitigate the opioid's effect on central (brain and spinal cord) opioid receptors. Consequently, these characteristics indicate that the compounds will also be substantially free of other centrally mediated effects. Other conditions that may be treated or prevented with compounds of the present invention, and techniques for formulating and administering such compounds, are described for example, in co-pending U.S. application Ser. No. 09/725,708, filed Nov. 29, 2000, now allowed, and co-pending U.S. application Ser. No. 09/725,661, filed Nov. 29, 2000, now allowed, the disclosures of each of which are hereby incorporated herein by reference, in their entireties.

In certain preferred embodiments, compounds of the present invention are peripheral opioid antagonist compounds, and preferably, mu opioid antagonist compounds. The term peripheral designates that the compound acts primarily on physiological systems and components external to the central nervous system, i.e., the compound preferably does not readily cross the blood-brain barrier. In preferred form, the peripheral opioid antagonist compounds employed in the methods of the present invention exhibit high levels of activity with respect to gastrointestinal tissue, while exhibiting reduced, and preferably substantially no, central nervous system (CNS) activity. The term “substantially no CNS activity”, as used herein, means that less than about 20% of the pharmacological activity of the peripheral opioid antagonist compounds employed in the present methods is exhibited in the CNS. In preferred embodiments, the peripheral opioid antagonist compounds employed in the present methods exhibit less than about 15% of their pharmacological activity in the CNS, with less than about 10% being more preferred. In even more preferred embodiments, the peripheral opioid antagonist compounds employed in the present methods exhibit less than about 5% of their pharmacological activity in the CNS, with about 0% (i.e., no CNS activity) being still more preferred.

Accordingly, embodiments of the present invention are directed to pharmaceutical compositions involving mu opioid antagonist compounds, as well as methods involving the administration to a patient of a mu opioid antagonist compound. The methods of the present invention may be used to treat patients who are also being administered compounds that may slow gut motility including, for example, opiates and/or opioids, such as opioid analgesics, prior to, during, and subsequent to the onset of ileus. The administration of such opiate or opioid compounds may induce bowel dysfunction which, in turn, may delay recovery from ileus, including postoperative ileus. The methods of the present invention may also be used to treat patients who have not received any exogenous opiates and/or opioids. Thus, in certain embodiments, the present methods comprise administering a compound to patients who have not received any opioid analgesic drugs including, for example, any mu opioid agonists.

Compounds as described herein may be administered by any means that results in the contact of the active agent(s) with the agents' sit or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

The compounds are preferably combined with a pharmaceutical carrier selected on the basis of the chosen route of administration and standard pharmaceutical practice as described, for example, in Remington's Pharmaceutical Sciences (Mack Pub. Co., Easton, Pa., 1980), the disclosures of which are hereby incorporated herein by reference, in their entirety.

Compounds of the present invention can be administered to a mammalian host in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol and rectal systemic.

The active compound may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained. Preferred compositions or preparations according to the present invention may be prepared so that an oral dosage unit form contains from about 0.1 to about 1000 mg of active compound.

Tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

The active compound may also be administered parenterally or intraperitoneally. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols and mixtures thereof, and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and the freeze drying techniques which yield a powder of the active ingredient, plus any additional desired ingredient from the previously sterile-filtered solution thereof.

The therapeutic compounds of this invention may be administered to a patient alone or in combination with a pharmaceutically acceptable carrier. As noted above, the relative proportions of active ingredient and carrier may be determined, for example, by the solubility and chemical nature of the compounds, chosen route of administration and standard pharmaceutical practice.

The dosage of the compounds of the present invention that will be most suitable for prophylaxis or treatment will vary with the form of administration, the particular compound chosen and the physiological characteristics of the particular patient under treatment. Generally, small dosages may be used initially and, if necessary, increased by small increments until the desired effect under the circumstances is reached. Generally speaking, oral administration may require higher dosages.

Although the proper dosage of compounds of this invention will be readily ascertainable by one skilled in the art, once armed with the present disclosure, by way of general guidance, for example, typically a daily dosage may range from about 0.001 to about 100 milligrams of the peripheral opioid antagonist (and all combinations and subcombinations of ranges and specific dosages therein), per kilogram of patient body weight. Preferably, a daily dosage may be from about 0.01 to about 10 milligrams of the opioid antagonist per kilogram of patient body weight.

### EXAMPLES

The invention is further described in the following examples (also see FIG. 1). All of the examples are actual examples. These examples are for illustrative purposes only, and are not to be construed as limiting the appended claims.

### Example 1

### Baylis-Hillman Reaction¹

¹ A recent example from patent literature uses 20 mol % DABCO/7d/rt in the coupling of butyraldehyde with methyl acrylate. The product was isolated in 61% yield; U.S. Pat. No. 74,522,999B2. Other examples of Baylis-Hillman reactions include Perlmutter, et al, J. Org. Chem., 1995, 60, 6515,Perlmutter, et al., Tetrahedron Left., 1988, 29, 949, Organic Syntheses, Vol. 75, p. 106 (1998), Amos B. Smith III, Ed.; and Lee et al., Tetrahedron Letters, Volume 40, Issue 23, 4 Jun. 1999, Pages 4363-4366 the disclosures of which are hereby incorporated herein by reference in their entireties.

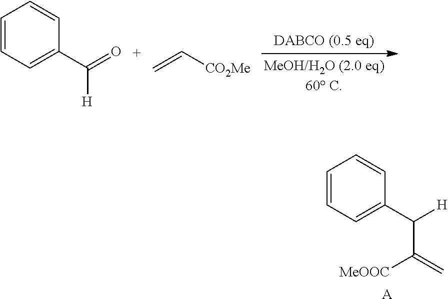

Into a reaction apparatus including a 30 L glass reactor and a 30 L extraction vessel was introduced 2.1 kg benzaldehyde, 5.3 L methyl acrylate, 0.3 kg of DABCO, 1.6 L methanol, and 0.7 L of water. The reaction was heated to 60° C. for 48 hours, at which time about 75% of the benzaldehyde was converted. An additional 0.2 kg of DABCO was added and heating was continued at 60° C. for another 24 hours. Excess methyl acrylate was removed by distillation at reduced pressure (60-65 C/200 mbar). The remaining crude was diluted with toluene and the solution was washed several times with aqueous sodium sulphite solution. The organics were then evaporated to dryness to provide Compound A (1.85 kg; 49% yield, 90% g.c. purity)

### Example 2

### Baylis-Hillman Reaction

Into a reaction apparatus including a 30 L glass reactor and a 30 L extraction vessel was introduced benzaldehyde (1.90 L, 1.99 kg, 18.7 mol, 1.0 eq), methyl acrylate (5.06 L, 4.83 kg, 56.1 mol, 3.0 eq), DABCO(1.05 kg, 9.4 mol, 0.5 eq), 1.51 L methanol, and 0.67 L of water under an inert atmosphere. The reaction was heated to 60° C. for 48 hours, at which time about 85% of the benzaldehyde was converted. An additional 0.19 kg of methyl acrylate was added and heating was continued at 60° C. for another 24 hours. Toluene (7 L) was added followed by NaHSO₃ solution (38%, 3.0 L). The temperature rose from 25 to 34° C. After agitation and separation, the layer NaHSO₃ was removed and the organics were contacted with fresh NaHSO₃ solution (38%, 3.0 L). After separation the remaining organic crude was washed several times with water and subsequently with brine. The organics were then evaporated to dryness (40 C/50 mbar) to provide Compound A (2.52 kg; 60% yield, 85% g.c. purity

### Example 3

### Acylation/Rearrangement

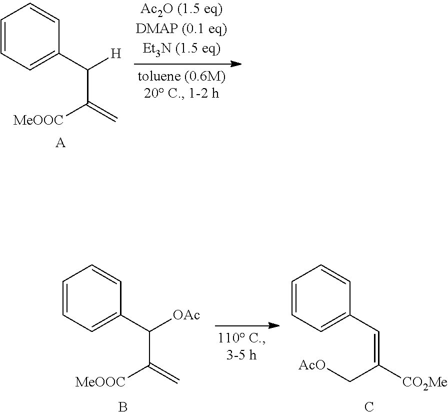

Into a reaction apparatus including a 30 L glass reactor and a 50 L extraction vessel was introduced Compound A (1.85 kg, 9.6 mol, 1.0 eq), triethylamine (2.0 L, 1.46 kg, 14.4 mol, 1.5 eq), DMAP (0.12 kg, 0.9 mol, 0.1 eq) and 16 L toluene under an inert atmosphere. The reaction was maintained at a temperature of 22-25° C. for one hour while acetic anhydride (1.47 kg) was added. The reaction temperature was held at 22-25° C. for an additional hour and subsequently heated to reflux (˜110° C.) for 4 hours. The mixture was slowly cooled to RT. The organics were washed twice with 0.5N HCl (8 L) with cooling to counter the exotherm, once with sat'd. NaHCO₃ ((7.0 L) and brine (8.0 L), and the toluene was evaporated to provide Compound C (1.9 kg, 85% purity, 72%, yield, approx. 93/7 E/Z as determined by ¹H-NMR, which is in agreement with reports from literature.²). The crude product was short path distilled (0.03 mbar/136-139° C.) to a yellow oil. ² Tetrahedron Lett. 2003, 44, 4673.

### Example 4

### Alkylation of trans 3(R),4-dimethyl-4(R)-(3-hydroxyphenyl)piperidine

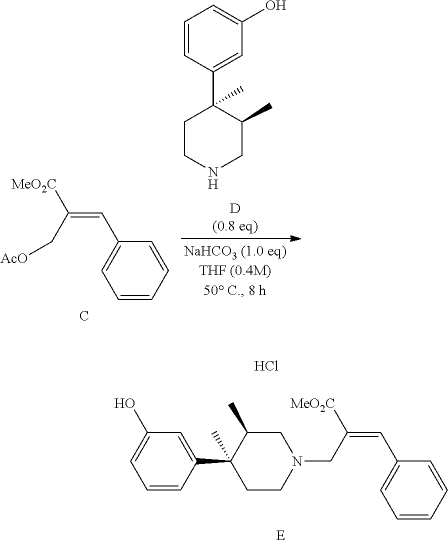

Into a reaction apparatus including a 30 L glass reactor and a suction filter was introduced Compound C (1.81 kg, 7.72 mol, 1.0 eq), Compound D³ (1.27 kg, 6.18 mol, 0.8 eq), and NaHCO₃ (0.55 kg, 6.49 mol, 0.85 eq) in 15.0 L THF. The reaction was heated to 50° C. for 20 hours under an inert atmosphere. The initially heterogeneous reaction slowly turned homogeneous over the course of the reaction. The crude reaction mixture was cooled to RT and filtered to remove the NaHCO₃. The filtrate was concentrated to half its initial volume (to approx. 12.5 L) and an equal volume of heptane was added (˜12.51L). The mixture was cooled to 0-5° C. and HCl gas (0.26 kg, 7.13 mol, 1.2 eq) was added with cooling to maintain the temperature and stirred for approx. 3.5 hours after HCl addition was completed. Compound E was precipitated as its HCl salt. During filtration of the salt, the product congealed. After rinsing, the crude product was dried for 12 hours and pestled. The yield of Compound E was 2.90 kg, 106%, 94% purity (HPLC), which was hygroscopic at this stage. The free base, which was not isolated and used as is in the hydrogenation step, could be recovered by liberation with aqueous NaHCO₃ and purified by flash chromatography. ³ Compound D may be synthesized using the method described in Werner et al., J Org. Chem., 1996, 61, 587.

### Example 5

### Ester Hydrolysis

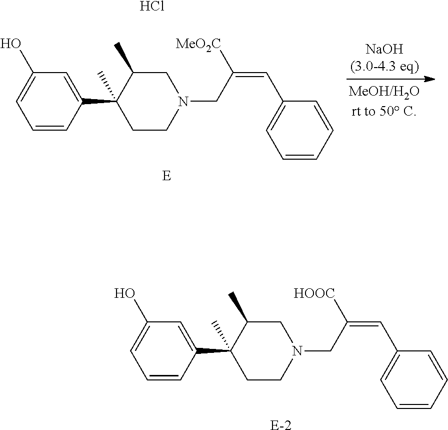

Into a reaction apparatus including a 20 L glass reactor and a 50 L extraction vessel was introduced water with cooling to 10 C. NaOH (0.89 kg, 22.2 mol, 4.0 eq) was added and dissolved under an inert atmosphere. Compound E (2.58 kg, 5.52 mol, 1.0 eq) was dissolved in 4.5 L MeOH and slowly added over 40 minutes while maintaining the temperature at less than 38 C. The reaction was heated for 1.5 hours/50° C. The mixture was cooled to room temperature and neutralized by slow addition of conc. H₂SO₄ (0.41 L, 7.31 mol, 1.3 eq.) until a pH of 7.0 was obtained. pH 7 buffer(3.0 L pH 7-buffer) was added slowly; some brine (1 L) was added to limit the amount of water that was extracted into organic phase. Mixture was extracted with EtOAc (3× 5 L). The organic phases were combined, dried from Na₂SO₄ (0.5 kg Na₂SO₄), and filtered over a plug of SiO₂ (1.0 kg SiO₂). The filtrate was evaporated, dried by repeated addition/removal of a mixture of methanol and ETOAc (0.2 L/4.0 L) and re-crystallized from hot acetone (10 L). The yield of Compound E-2 after vacuum drying (40 C/200 mbar) was 1.32 kg, 64%, >98% purity, E/Z 97:3).

### Example 6

### Asymmetric Hydrogenation with Rhodium Catalysts

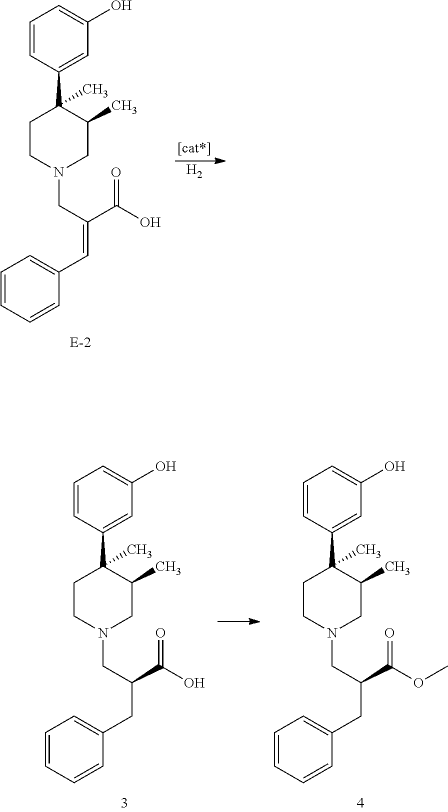

A broad screening of the asymmetric hydrogenation using a representative selection of Rh catalysts was performed with pure E-2, the results of which are summarized in Table 1.

TABLE 1
Asymmetric hydrogenation of E-2. Screening of Rh catalysts

| | | | | | | | | | d.r. |
|---|---|---|---|---|---|---|---|---|---|
| | | | | | | | | | (αR, 3R, 4R) |
| | | | | | | | | | isomer |
| | | | | | | | | | Compound 4′ |
| | | | | | | | | | vs. |
| | | | | | | | | Con- | (αS, 3R, 4R) |
| Entry | | | Ligand | Abs. | Ligand | p(H₂) | Temp. | version | isomer |
| No. | a) | Precursor | #SL- | Config. | Acronym | (bar) | (° C.) | (%) | Compound 4 |
| 1 | E | [Rh(nbd)₂]BF₄ | J504-1 | (R)-(S) | cy₂PF—P(oTol)₂ | 80 | 25 | 99.5 | 4:96 |
| 2 | E | [Rh(nbd)₂]BF₄ | J505-1 | (R)-(S) | tBu₂PF—P(oTol)₂ | 80 | 25 | 97 | 6:94 |
| 3 | E | [Rh(nbd)₂]BF₄ | J503-1 | (R)-(S) | Et₂PF—P(oTol)₂ | 80 | 25 | 98 | 9:91 |
| 4 | E | [Rh(nbd)₂]BF₄ | J506-1 | (R)-(S) | tBu₂PF—P(4-CF₃Ph)₂ | 80 | 25 | 99 | 9:91 |
| 5 | E | [Rh(nbd)₂]BF₄ | M004-2 | (S)-(R) | MOD-mandyphos | 80 | 25 | 100 | 9.3:90.7 |
| 6 | E | [Rh(nbd)₂]BF₄ | W005-2 | (S)-(S) | (3,5-Me-4-MeOPh)₂P— | 80 | 25 | 100 | |
| | | | | | PhFc-CH—(CH₃)P(3,5- | | | | 10.5:89.5 |
| | | | | | CF₃)₂ | | | | |
| 7 | E | [Rh(nbd)₂]BF₄ | W001-2 | (S)-(S) | Ph₂PPhFc-CH— | 80 | 25 | 100 | 10.6:89.4 |
| | | | | | (CH₃)P(3,5-CF₃)₂ | | | | |
| 8 | E | [Rh(nbd)₂]BF₄ | W009-2 | (S)-(S) | xyl₂PPhFc-CH— | 80 | 25 | 100 | 13.7:86.3 |
| | | | | | (CH₃)Pxyl₂ | | | | |
| 9 | E | [Rh(nbd)₂]BF₄ | J502-1 | (R)-(S) | tBu₂PF—PPh₂ | 80 | 25 | 99 | 19:81 |
| 10 | E | [Rh(nbd)₂]BF₄ | J501-1 | (R)-(S) | tBu₂PF—Pxyl₂ | 80 | 25 | 76 | 20:80 |
| 11 | E | [Rh(nbd)₂]BF₄ | M002-2 | (S)-(R) | Pcy2-Mandyphos | 80 | 25 | 100 | 24.6:75.4 |
| 12 | E | [Rh(nbd)₂]BF₄ | W008-2 | (S)-(S) | cy₂PPhFc-CH— | 80 | 25 | 100 | 25:75 |
| | | | | | (CH₃)P(3,5-CF₃)₂ | | | | |
| 13 | E | [Rh(nbd)₂]BF₄ | A109-2 | (S) | 3,5-tBu-4-MeO— | 80 | 25 | 60 | 26:74 |
| | | | | | MeObiphep | | | | |
| 14 | E | [Rh(nbd)₂]BF₄ | A131-1 | (R) | biphemp | 80 | 25 | 34 | 27:73 |
| 15 | E | [Rh(nbd)₂]BF₄ | T001-1 | (R)-(S) | Taniaphos | 80 | 25 | 99 | 28:72 |
| 16 | E | [Rh(nbd)₂]BF₄ | J216-1 | (R)-(S) | (1-Naphtyl)₂PF—PtBu₂ | 80 | 25 | 100 | 29:71 |
| 17 | E | [Rh(nbd)₂]BF₄ | A101-1 | (R) | MeObiphep | 80 | 25 | 36 | 31:69 |
| 18 | E | [Rh(nbd)₂]BF₄ | W012-1 | (S)-(S) | Ph₂PPhFc-CH— | 80 | 25 | 100 | 38:62 |
| | | | | | (CH₃)PtBu₂ | | | | |
| 19 | E | [Rh(nbd)₂]BF₄ | J226-1 | (R)-(S) | (2-iPrOPhl)₂PF—PtBu₂ | 80 | 25 | 100 | 40:60 |
| 20 | E | [Rh(nbd)₂]BF₄ | J013-1 | (R)-(S) | (3,5-Me-4-MeOPh)₂PF— | 80 | 25 | 99 | 45:55 |
| | | | | | PtBu₂ | | | | |
| 21 | E | [Rh(nbd)₂]BF₄ | J510-1 | (R)-(S) | (Me)(tBu)PF—Pxyl₂ | 80 | 25 | 55 | 46:54 |
| 22 | E | [Rh(nbd)₂]BF₄ | J014-1 | (R)-(S) | (4-FPh)₂PF—PtBu₂ | 80 | 25 | 99 | 47.5:52.5 |
| 23 | E | [Rh(nbd)₂]BF₄ | M031-2 | (S)-(R) | ferriphos | 80 | 25 | 95 | 48:52 |
| 24 | E | [Rh(nbd)₂]BF₄ | J219-1 | (R)-(S) | (2-Anisyll)₂PF—PtBu₂ | 80 | 25 | 100 | 50:50 |
| 25 | E | [Rh(nbd)₂]BF₄ | T021-2 | (S)-(S) | Hydroxy-Taniaphos | 80 | 25 | 100 | 55:45 |
| 26 | E | [Rh(nbd)₂]BF₄ | T025-2 | (S)-(S) | (2,2′-Pxyl₂)-Hydroxy- | 80 | 25 | 100 | 57:43 |
| | | | | | Taniaphos | | | | |
| 27 | E | [Rh(nbd)₂]BF₄ | J404-2 | (S)-(R) | (1-Naphtyl)₂PF—Pxyl₂ | 80 | 25 | 100 | 66:34 |
| 28 | E | [Rh(nbd)₂]BF₄ | J216-2 | (S)-(R) | (1-Naphtyl)₂PF—PtBu₂ | 80 | 25 | 100 | 72:28 |

Reaction conditions: E-2:: 100 mg; s/c: 50; solvent MeOH 4 ml; time: 17 hrs. Additive: 0.5 eq MsOH
d.r. means diastereomeric ratio
^{a)} E-isomer: The stereochemistry at the C═C bond was confirmed by ¹H-NMR (NOE). The E-2 isomer was stereochemically pure (¹H-NMR)
^{b)} The last digit in the ligand identifier indicates whether the ligand is enantiomer 1 or enantiomer 2 (i.e., J404-1 and J404-2 are enantiomers of one another. It follows that if J404-1 has an (R)-(S) configuration, then J404-2 has an (S)-(R) configuration). Chiral phosphine ligands, such as those identified in the hydrogenation tables, are available from Solvias AG, Römerpark 2, 4303 Kaiseraugst, Switzerland.

### Diastereomers of Compounds 3 and 4

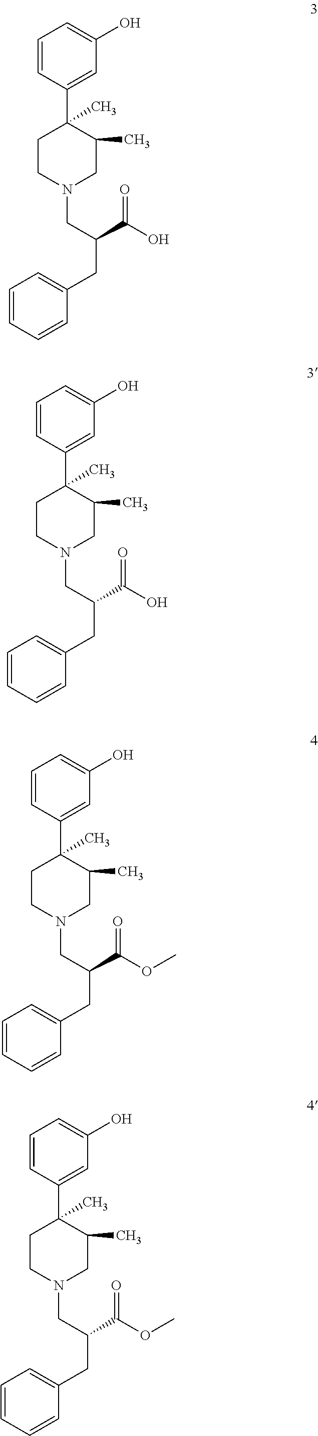 [SMILES: C[C@H]1CN(C[C@H](Cc2ccccc2)C(=O)O)CC[C@@]1(C)c1cccc(O)c1 ; C[C@H]1CN(C[C@@H](Cc2ccccc2)C(=O)O)CC[C@@]1(C)c1cccc(O)c1 ; COC(=O)[C@@H](Cc1ccccc1)CN1CC[C@@](C)(c2cccc(O)c2)[C@@H](C)C1 ; COC(=O)[C@H](Cc1ccccc1)CN1CC[C@@](C)(c2cccc(O)c2)[C@@H](C)C1]

### Example 7

### Asymmetric Hydrogenation with Ruthenium Catalysts

Screening of Ru Diphosphine Catalysts

Two types of precursors, [Ru(cod)(2-metallyl)₂] and [RuI₂(p-cymene)]₂ were used for the in situ formation of the catalysts. Twenty five chiral hydrogenation screening experiments were performed and the results are compiled in Table 2.

TABLE 2
Asymmetric hydrogenation of E-2. Screening of Ru catalysts

| | | | | | | | | | | d.r. |
|---|---|---|---|---|---|---|---|---|---|---|
| | | | | | | | | | | (αR,3R,4R) |
| | | | | | | | | | | isomer |
| | | | | | | | | | | Compound 4′ |
| | | | | | | | | | | vs. |
| | | | | | | | | | Con- | (αS,3R,4R) |
| Entry.- | | | Ligand | abs. | | | p(H₂) | Temp. | version | isomer |
| No. | a) | Precursor | #SL- | Config. | Acronym | | (bar) | (° C.) | (%) | Compound 4 |
| 29 | E | [Ru(cod)(metallyl)₂] | W001-2 | (S)-(S) | Ph₂PPhFc-CH— | MsOH | 50 | 40 | 98 | 18:82 |
| | | | | | (CH₃)P(3,5-CF₃)₂ | (0.5) | | | | |
| 30 | E | [Ru(p-cymene)I₂] | T001-1 | (R)-(S) | taniaphos | KOH | 50 | 40 | 20 | 31:69 |
| | | | | | | (0.9)/ | | | | |
| | | | | | | NEt₃ | | | | |
| | | | | | | (1.1) | | | | |
| 31 | E | [Ru(p-cymene)I₂] | J212-1 | (R)-(S) | (2-Furyl)₂PF—PtBu₂ | MsOH | 50 | 40 | 96 | 32:68 |
| | | | | | | (0.5) | | | | |
| 32 | E | [Ru(cod)(metallyl)₂] | J212-1 | (R)-(S) | (2-Furyl)₂PF—PtBu₂ | MsOH | 50 | 40 | 90 | 34:66 |
| | | | | | | (0.5) | | | | |
| 33 | E | [Ru(p-cymene)I₂] | J503-2 | (S)-(R) | Et₂PF—P(oTol)₂ | NEt₃ | 50 | 40 | 16 | 35:65 |
| | | | | | | (1.0) | | | | |
| 34 | E | [Ru(p-cymene)I₂] | M004-2 | (S)-(R) | MOD-mandyphos | NEt₃ | 50 | 40 | 11 | 42:58 |
| | | | | | | (1.0) | | | | |
| 35 | E | [Ru(p-cymene)I₂] | J002-1 | (R)-(S) | PPF—PtBu₂ | MsOH | 50 | 40 | 92 | 43:57 |
| | | | | | | (0.5) | | | | |
| 36 | E | [Ru(p-cymene)I₂] | W005-2 | (S)-(S) | (3,5-Me-4-MeOPh)₂P— | NEt₃ | 50 | 40 | 17 | 46:54 |
| | | | | | PhFc-CH—(CH₃)P(3,5- | (1.0) | | | | |
| | | | | | CF₃)₂ | | | | | |
| 37 | E | [Ru(p-cymene)I₂] | W005-2 | (S)-(S) | (3,5-Me-4-MeOPh)₂P— | NEt₃ | 50 | 40 | 17 | 46:54 |
| | | | | | PhFc-CH—(CH₃)P(3,5- | (1.0) | | | | |
| | | | | | CF₃)₂ | | | | | |
| 38 | E | [Ru(p-cymene)I₂] | A001-2 | (S) | solphos | TMG | 50 | 40 | 12 | 49:51 |
| | | | | | | (1.0) | | | | |
| 39 | E | [Ru(p-cymene)I₂] | A131-2 | (S) | biphemp | NEt₃ | 50 | 40 | 12 | 70:30 |
| | | | | | | (1.0) | | | | |
| 40 | E | [Ru(p-cymene)I2] | A101-2 | (S) | MeObiphep | NEt₃ | 50 | 40 | 19 | 72:28 |
| | | | | | | (1.0) | | | | |
| 41 | E | [Ru(cod)(metallyl)₂] | T025-2 | (S)-(S) | (2,2′-Pxyl₂)-Hydroxy- | MsOH | 50 | 40 | 78 | 75:25 |
| | | | | | Taniaphos | (0.5) | | | | |
| 42 | E | [Ru(cod)(metallyl)₂] | A101-1 | (R) | MeObiphep | MsOH | 50 | 40 | 41 | 76:42 |
| | | | | | | (0.5) | | | | |
| 43 | E | [Ru(p-cymene)I₂] | A101-1 | (R) | MeObiphep | MsOH | 50 | 40 | 23 | 89:11 |
| | | | | | | (0.5) | | | | |
| 44 | E | [Ru(p-cymene)I₂] | J005-1 | (R)-(S) | PPF—Pxyl₂₋ | MsOH | 50 | 40 | <1 | n.d. |
| | | | | | | (0.5) | | | | |
| 45 | E | [Ru(p-cymene)I₂] | A101-2 | (S) | MeObiphep | TMG | 50 | 40 | <5 | n.d. |
| | | | | | | (1.0) | | | | |
| 46 | E | [Ru(p-cymene)I₂] | A109-2 | (S) | 3,5-tBu-4-MeO— | NEt₃ | 50 | 40 | <5 | n.d. |
| | | | | | MeObiphep | (1.0) | | | | |
| 47 | E | [Ru(p-cymene)I₂] | A109-2 | (S) | 3,5-tBu-4-MeO— | TMG | 50 | 40 | <5 | n.d. |
| | | | | | MeObiphep | (1.0) | | | | |
| 48 | E | [Ru(p-cymene)I₂] | A001-2 | (S) | solphos | NEt₃ | 50 | 40 | <5 | n.d. |
| | | | | | | (1.0) | | | | |
| 49 | E | [Ru(p-cymene)I₂] | A101-2 | (S) | MeObiphep | KOH | 50 | 40 | <5 | n.d. |
| | | | | | | (0.9)/ | | | | |
| | | | | | | NEt₃ | | | | |
| | | | | | | (1.1) | | | | |
| 50 | E | [Ru(p-cymene)I₂] | M004-2 | (S)-(R) | MOD-mandyphos | KOH | 50 | 40 | <5 | n.d. |
| | | | | | | (0.9)/ | | | | |
| | | | | | | NEt₃ | | | | |
| | | | | | | (1.1) | | | | |
| 51 | E | [Ru(p-cymene)I₂] | W012-1 | (R)-(R) | Ph₂PPhFc-CH— | KOH | 50 | 40 | <5 | n.d. |
| | | | | | (CH₃)PtBu₂ | (0.9)/ | | | | |
| | | | | | | NEt₃ | | | | |
| | | | | | | (1.1) | | | | |
| 52 | E | [Ru(p-cymene)I₂] | A001-2 | (S) | solphos | KOH | 50 | 40 | <5 | n.d. |
| | | | | | | (0.9)/ | | | | |
| | | | | | | NEt₃ | | | | |
| | | | | | | (1.1) | | | | |

### Example 8

### Asymmetric Hydrogenation of E-2. Screening of Additives

Additional experiments were performed with Rh(nbd)₂]BF₄ and SL-J505-1 to evaluate the effect of catalyst load, the influence of additives (acids and bases), temperature and pressure. These results are shown in Table 3.

TABLE 3
Asymmetric hydrogenation of E-2 by means of Rh/SL-J505-l. Screening of additives

| | | | | | | | | | | d.r. |
|---|---|---|---|---|---|---|---|---|---|---|
| | | | | | | | | | | (αR,3R,4R) |
| | | | | | | | | | | isomer |
| | | | | | | | | | | Compound 4′ |
| | | | | | | | | | | vs. |
| | | s.m. | | | | | | | Con- | (αS,3R,4R) |
| Entry.- | | (Substrate) | | solvent | | eq/ | p(H₂) | Temp. | version | isomer |
| No. | | g (mmol) | s/c | (ml) | Additive | s.m. | (bar) | (° C.) | (%) | Compound 4 |
| 53 | E | 0.1 | 50 | MeOH (4) | MsOH | 1.0 | 80 | 25 | 99 | 2.5:97.5 |
| | | (0.2736) | | | | | | | | |
| 54 | E | 0.1 | 50 | MeOH (4) | HCl (1N, aq) | 0.5 | 80 | 25 | 99.5 | 3.5:96.5 |
| | | (0.2736) | | | | | | | | |
| 55 | E | 0.1 | 50 | MeOH (4) | H₂SO₄ | 0.5 | 80 | 25 | 99 | 4.5:95.5 |
| | | (0.2736) | | | | | | | | |
| 56 | E | 0.1 | 50 | MeOH (4) | MsOH | 0.5 | 80 | 25 | 97 | 6:94 |
| | | (0.2736) | | | | | | | | |
| 57 ^{a)} | E | 0.2 | 100 | MeOH (8) | MsOH | 0.5 | 50 | 25 | 99.3 | 2.4:97.6 |
| | | (0.5472) | | | | | | | | |
| 58 ^{a)} | E | 0.2 | 100 | MeOH (8) | MsOH | 0.5 | 50 | 40 | 98.6 | 2.9:97.1 |
| | | (0.5472) | | | | | | | | |
| 59 | E | 0.2 | 200 | MeOH (4) | none | — | 20 | 25 | 30 | 11:89 |
| | | (0.5472) | | | | | | | | |
| 60 | E | 0.2 | 200 | MeOH (4) | NEt₃ | 0.5 | 20 | 25 | 6 | 12:87 |
| | | (0.5472) | | | | | | | | |
| 61 | E | 0.2 | 200 | MeOH (4) | MsOH | 0.5 | 20 | 25 | 49.5 | 12:88 |
| | | (0.5472) | | | | | | | | |
| 62 | E | 0.2 | 200 | MeOH (4) | MsOH | 0.5 | 20 | 25 | 16 | 16:84 |
| | | (0.5472) | | | | | | | | |
| 63 | E | 0.2 | 200 | MeOH (4) | AcOH | 1.0 | 20 | 25 | 11 | 27:73 |
| | | (0.5472) | | | | | | | | |
| 64 | E | 0.2 | 200 | MeOH (4) | H₂SO₄ | 1.0 | 20 | 25 | 91 | 3.2:96.8 |
| | | (0.5472) | | | | | | | | |
| 65 | E | 0.2 | 200 | MeOH (4) | HCl (1N, aq) | 1.0 | 20 | 25 | 17 | 3.3:96.7 |
| | | (0.5472) | | | | | | | | |
| 66 | E | 0.2 | 200 | MeOH (4) | KOH | 0.95 | 20 | 25 | 49 | 4:96 |
| | | (0.5472) | | | | | | | | |
| 67 | E | 0.2 | 200 | MeOH (4) | H₂SO₄ | 0.5 | 20 | 25 | 91 | 5:95 |
| | | (0.5472) | | | | | | | | |
| 68 | E | 0.2 | 200 | MeOH (4) | MsOH | 1.0 | 20 | 25 | 5 | 6:94 |
| | | (0.5472) | | | | | | | | |
| 69 | E | 0.2 | 200 | MeOH (4) | none | — | 20 | 25 | 68.5 | 7:93 |
| | | (0.5472) | | | | | | | | |
| 70 | E | 0.3 | 300 | MeOH (8) | H₂SO₄ | 0.5 | 20 | 40 | 96 | 3:97 |
| | | (0.8208) | | | | | | | | |
| 71 | E | 0.3 | 300 | iPrOH (8) | H₂SO₄ | 0.5 | 20 | 50 | 63 | 6:94 |
| | | (0.8208) | | | | | | | | |
| 72 *⁾ | E | 0.3 | 500 | MeOH (8) | HCl (1N, aq) | 1.0 | 20 | 50 | 0 | — |
| | | (0.8208) | | | | | | | | |
| 73 *⁾ | E | 0.3 | 500 | MeOH (8) | H₂SO₄ | 1.0 | 20 | 50 | 0 | — |
| | | (0.8208) | | | | | | | | |
| 74 | E | 1.0 | 500 | MeOH (15) | none | — | 20 | 35 | 0 | — |
| | | (2.74) | | | | | | | | |
| 75 | E | 1.0 | 500 | MeOH (15) | H₂SO₄ | 0.5 | 20 | 40 | 97.3 | 12:88 |
| | | (2.74) | | | | | | | | |
| 76 *⁾ | E | 0.3 | 500 | MeOH (8) | H₂SO₄ | 0.5 | 20 | 50 | 4 | 6:94 |
| | | (0.8208) | | | | | | | | |
| 77 *⁾ | E | 0.3 | 500 | MeOH (8) | HCl (1N, aq) | 0.5 | 20 | 50 | 1 | n.d |
| | | (0.8208) | | | | | | | | |
| 78 | E | 1.0 | 1000 | MeOH (15) | H₂SO₄ | 0.5 | 20 | 35 | 84.5 | 11:89 |
| | | (2.74) | | | | | | | | |
| 79 | E | 0.75 | 1000 | MeOH (8) | H₂SO₄ | 1.0 | 20 | 60 | 63 | 5:95 |
| | | (2.05) | | | | | | | | |
| 80 | E | 1.5 | 2000 | MeOH (20) | H₂SO₄ | 0.5 | 20 | 40 | 16.5 | 5.4:94.6 |
| | | (4.1) | | | | | | | | |

Reaction conditions: Catalyst: [Rh(nbd)₂]BF₄ + SL-J505-1; Time: 15-19 hrs.
*⁾ No conversion because of problematic experimental setup

Additional screening experiments were also carried out with Rh/SL-J504-1. Results of this experimental series are summarized in Table 4.

TABLE 4
Asymmetric hydrogenation of E-2 by means of Rh/SL-J504-l. Screening of additives

| | | | | | | | | | | d.r. |
|---|---|---|---|---|---|---|---|---|---|---|
| | | | | | | | | | | (αR,3R,4R) |
| | | | | | | | | | | isomer |
| | | | | | | | | | | Compound 4′ |
| | | | | | | | | | | vs. |
| | | s.m. | | | | | | | Con- | (αS,3R,4R) |
| Entry.- | | (Substrate) | | solvent | | eq/ | p(H₂) | Temp. | version | isomer |
| No. | | g (mmol) | s/c | (ml) | Additive | s.m. | (bar) | (° C.) | (%) | Compound 4 |
| 81 | E | 0.1 | 50 | MeOH (4) | MsOH | 0.5 | 80 | 25 | 99.5 | 4:96 |
| | | (0.2736) | | | | | | | | |
| 82 | E | 0.2 | 200 | MeOH (4) | MsOH | 0.5 | 20 | 25 | 98 | 4:96 |
| | | (0.5472) | | | | | | | | |
| 83 | E | 0.2 | 200 | MeOH (4) | MsOH | 1.0 | 20 | 25 | <5 | n.d. |
| | | (0.5472) | | | | | | | | |
| 84 | E | 0.2 | 200 | MeOH (4) | none | — | 20 | 25 | 96 | 5.7:94.3 |
| | | (0.5472) | | | | | | | | |
| 85 | E | 0.2 | 200 | MeOH (4) | H₂SO₄ | 0.5 | 20 | 25 | 98 | 4.7:95.3 |
| | | (0.5472) | | | | | | | | |
| 86 | E | 0.2 | 200 | MeOH (4) | H₂SO₄ | 1.0 | 20 | 25 | 96 | 3.5:96.5 |
| | | (0.5472) | | | | | | | | |
| 87 | E | 0.3 | 300 | MeOH (8) | H₂SO₄ | 0.5 | 20 | 40 | 98.5 | 4.7:95.3 |
| | | (0.8208) | | | | | | | | |
| 88 *⁾ | E | 0.3 | 500 | MeOH (8) | HCl (1N, aq) | 0.5 | 20 | 50 | 0 | — |
| | | (0.8208) | | | | | | | | |
| 89 *⁾ | E | 0.3 | 500 | MeOH (8) | HCl (1N, aq) | 1.0 | 20 | 50 | 0 | — |
| | | (0.8208) | | | | | | | | |
| 90 *⁾ | E | 0.3 | 500 | MeOH (8) | H₂SO₄ | 0.5 | 20 | 50 | 4 | 8:92 |
| | | (0.8208) | | | | | | | | |
| 91 *⁾ | E | 0.3 | 500 | MeOH (8) | H₂SO₄ | 1.0 | 20 | 50 | 0 | — |
| | | (0.8208) | | | | | | | | |
| 92 *⁾ | E | 0.75 | 1000 | MeOH (8) | H₂SO₄ | 1.0 | 20 | 60 | 0 | — |
| | | (2.05) | | | | | | | | |
| 93 | E | 1.0 | 500 | MeOH (15) | H₂SO₄ | 0.5 | 20 | 40 | 99 | 5:95 |
| | | (2.74) | | | | | | | | |
| 94 | E | 1.0 | 500 | MeOH (10) | H₂SO₄ | 0.5 | 20 | 40 | 100 | 5:95 |
| | | (2.74) | | | | | | | | |
| 95 | E | 1.0 | 750 | MeOH (15) | H₂SO₄ | 0.5 | 20 | 40 | 100 | 4.6:95.4 |
| | | (2.74) | | | | | | | | |
| 96 | E | 1.5 | 1000 | MeOH (20) | H₂SO₄ | 0.5 | 20 | 40 | >99.8 | 5.7:94.3 |
| | | (4.1) | | | | | | | | |
| 97 | E | 1.5 | 2000 | MeOH (20) | H₂SO₄ | 0.5 | 20 | 40 | >99.8 | 5.9:94.1 |
| | | (4.1) | | | | | | | | |
| 98 | E/Z | 1.0 | 750 | MeOH (15) | H₂SO₄ | 0.5 | 20 | 40 | 91 | 8.4:91.6 |
| | | (2.74) | | | | | | | | |
| 99 | E/Z | 1.5 | 2000 | MeOH (20) | H₂SO₄ | 0.5 | 20 | 40 | 74 | 5.4:94.6 |
| | | (4.1) | | | | | | | | |
| 100 ^{a)} | E | 1.5 | 2000 | MeOH (20) | H₂SO₄ | 0.5 | 20 | 40 | 98 | 5.3:94.7 |
| | | (4.1) | | | | | | | | |

Reaction conditions: Catalyst: [Rh(nbd)₂]BF₄ + SL-J504-1
^{a)}: [Rh(cod)₂]BF₄ + SL-J504-1).
*⁾ No conversion because of problematic experimental setup

### Example 9

Additional plated screening experiments were also carried out with a group of diphosphine liganded Group VIII metal catalysts in a range of solvents employing a variety of additives. Certain catalysts were prepared in situ from the identified diphosphine ligand and a catalyst precursor selected from [Rh(nbd)₂]BF₄, [Rh(cod)₂]OTf, or [Ru(OOCCF³)²(cod)]. Results of this experimental series are summarized in Tables 5 and 5A. The data in Table 5 include two columns of information listing the per cent of Compound 3 and percent of Compound 3′. The data in Table 5A express this information as a percent diastereomeric excess and employ positive and negative values for the percent diastereomeric excess to indicate whether Compound 3 or Compound 3′ is the product formed in excess in the stated hydrogenation reaction.

TABLE 5
Asymmetric Hydrogenation of E-2 Screening Results on HTS Plate

| Entry | | Ligand SL | | | | | |
|---|---|---|---|---|---|---|---|
| No. | Precursor | # | Additive | Solvent | Conv. | 3′:3 | Sel. |
| H | [Rh(NBD)₂]BF₄ | SL-F357-1 | | MeOH | 100 | 98.6:1.4 | 77.3 |
| G | [Rh(NBD)₂]BF₄ | SL-F356-1 | | MeOH | 100 | 97.2:2.8 | 73.3 |
| D | [Rh(NBD)₂]BF₄ | SL-F133-1 | | MeOH | 100 | 97.0:3.0 | 70.8 |
| H | [Rh(COD)₂]O₃SCF₃ | SL-F357-1 | TFA | 2-Me—THF | 99.1 | 94.6:5.4 | 76.8 |
| E | [Ru(COD)(OOCCF₃)₂] | SL-A120-2 | | MeOH | 100 | 93.5:6.5 | 50 |
| A | [Rh(NBD)₂]BF₄ | SL-C151-1 | | MeOH | 100 | 92.6:7.4 | 68.7 |
| C | [Rh(NBD)₂]BF₄ | SL-F132-1 | | MeOH | 100 | 91.8:8.2 | 68.2 |
| D | [Rh(NBD)₂]BF₄ | SL-M009-1 | H₂SO₄ | MeOH | 100 | 91.8:8.2 | 82.5 |
| A | [Rh(NBD)₂]BF₄ | SL-C151-1 | H₂SO₄ | MeOH | 100 | 91.4:8.6 | 86.3 |
| D | [Rh(NBD)₂]BF₄ | SL-M009-1 | | MeOH | 100 | 90:10 | 71 |
| D | [Rh(NBD)₂]BF₄ | SL-J686-1 | | MeOH | 100 | 80:10 | 74.2 |
| B | [Rh(COD)₂]O₃SCF₃ | SL-C020-2 | TFA | 2-Me—THF | 100 | 17.8 82.2 | 84.4 |
| C | [Rh(NBD)₂]BF₄ | SL-W024-1 | H₂SO₄ | MeOH | 100 | 17.3 82.7 | 85 |
| E | [Rh(NBD)₂]BF₄ | SL-C048-1 | H₂SO₄ | MeOH | 24 | 16 84 | 50.7 |
| C | [Rh(NBD)₂]BF₄ | SL-W024-1 | | MeOH | 100 | 14 86 | 76.7 |
| E | [Rh(NBD)₂]BF₄ | SL-T125-1 | | MeOH | 25.3 | 13.4 86.6 | 18.9 |

Reaction conditions: 4 mol % catalyst (s/c: 25); 10 bar; 25° C.; 16 hrs
The conversion and the diastereomeric ratio were determined by HPLC after derivatization of the crude product using TMS diazomethane. TFA = trifluoroacetic acid. TFE = 2,2,2-trifluoroethanol. Additives were provided to the reaction at a 0.5 eq additive per eq substrate level.

TABLE 5A
Asymmetric Hydrogenation of E-2 Screening Results on HTS Plate

| Entry | | Ligand | | | | | |
|---|---|---|---|---|---|---|---|
| No. | Precursor | SL# | Additive | Solvent | Conv. | % d.e. | Sel. |
| A | [Rh(NBD)₂]BF₄ | SL-A101-2 | | MeOH | 80.7 | 40.4 | 40.1 |
| A | [Rh(NBD)₂]BF₄ | SL-A101-2 | H₂SO₄ | MeOH | 100 | 39.1 | 85 |
| A | [Ru(COD)(OOCCF₃)₂] | SL-A101-2 | | MeOH | 65.1 | 80 | 15.3 |
| A | [Rh(NBD)₂]BF₄ | SL-F365-1 | | MeOH | 100 | −46.2 | 66.7 |
| A | [Rh(NBD)₂]BF₄ | SL-F365-1 | H₂SO₄ | MeOH | 100 | −25.3 | 84.2 |
| A | [Ru(COD)(OOCCF₃)₂] | SL-F365-1 | | MeOH | 100 | −24.6 | 49.3 |
| A | [Rh(NBD)₂]BF₄ | SL-F124-1 | | MeOH | 47 | 7.9 | 38.5 |
| A | [Rh(NBD)₂]BF₄ | SL-F124-1 | H₂SO₄ | MeOH | 13 | −50.7 | 48.6 |
| A | [Ru(COD)(OOCCF₃)₂] | SL-F124-1 | | MeOH | 89.2 | −5.1 | 24.1 |
| A | [Rh(NBD)₂]BF₄ | SL-C151-1 | | MeOH | 100 | 85.3 | 68.7 |
| A | [Rh(NBD)₂]BF₄ | SL-C151-1 | H₂SO₄ | MeOH | 100 | 83.1 | 86.3 |
| A | [Ru(COD)(OOCCF₃)₂] | SL-C151-1 | | MeOH | 65 | −40.5 | 20.2 |
| B | [Rh(NBD)₂]BF₄ | SL-A109-2 | | MeOH | 100 | −45 | 66.2 |
| B | [Rh(NBD)₂]BF₄ | SL-A109-2 | H₂SO₄ | MeOH | 100 | −42.8 | 82.2 |
| B | [Ru(COD)(OOCCF₃)₂] | SL-A109-2 | | MeOH | 100 | 56.7 | 50.6 |
| B | [Rh(NBD)₂]BF₄ | SL-J681-1 | | MeOH | 100 | 4 | 66.9 |
| B | [Rh(NBD)₂]BF₄ | SL-J681-1 | H₂SO₄ | MeOH | 100 | 0.2 | 75.2 |
| B | [Rh(COD)₂]O₃SCF₃ | SL-J681-1 | TFA | 2-Me—THF | 100 | −53.2 | 70.9 |
| B | [Rh(NBD)₂]BF₄ | SL-F131-1 | | MeOH | 32.7 | 47.4 | 13.6 |
| B | [Rh(NBD)₂]BF₄ | SL-F131-1 | H₂SO₄ | MeOH | 38.6 | 74 | 76.5 |
| B | [Rh(COD)₂]O₃SCF₃ | SL-F131-1 | TFA | 2-Me—THF | 17.3 | n.d. | 9.9 |
| B | [Rh(NBD)₂]BF₄ | SL-C020-2 | | MeOH | 100 | −62.7 | 74.6 |
| B | [Rh(NBD)₂]BF₄ | SL-C020-2 | H₂SO₄ | MeOH | 100 | −60.9 | 80.2 |
| B | [Rh(COD)₂]O₃SCF₃ | SL-C020-2 | TFA | 2-Me—THF | 100 | −64.4 | 84.4 |
| C | [Rh(NBD)₂]BF₄ | SL-A153-1 | | MeOH | 23.2 | n.d. | 2.4 |
| C | [Rh(NBD)₂]BF₄ | SL-A153-1 | H₂SO₄ | MeOH | 29.5 | −14.9 | 68.3 |
| C | [Ru(COD)(OOCCF₃)₂] | SL-A153-1 | | MeOH | 29.5 | n.d. | 4.4 |
| C | [Rh(NBD)₂]BF₄ | SL-J683-1 | | MeOH | 100 | 4 | 70.1 |
| C | [Rh(NBD)₂]BF₄ | SL-J683-1 | H₂SO₄ | MeOH | 100 | −3 | 79.1 |
| C | [Ru(COD)(OOCCF₃)₂] | SL-J683-1 | | MeOH | 52.3 | −21.8 | 41.3 |
| C | [Rh(NBD)₂]BF₄ | SL-F132-1 | | MeOH | 100 | 84.3 | 68.2 |
| C | [Rh(NBD)₂]BF₄ | SL-F132-1 | H₂SO₄ | MeOH | 33.8 | 41.7 | 74.3 |
| C | [Ru(COD)(OOCCF₃)₂] | SL-F132-1 | | MeOH | 47.3 | n.d. | 0.3 |
| C | [Rh(NBD)₂]BF₄ | SL-W024-1 | | MeOH | 100 | −72 | 76.7 |
| C | [Rh(NBD)₂]BF₄ | SL-W024-1 | H₂SO₄ | MeOH | 100 | −65.4 | 85 |
| C | [Ru(COD)(OOCCF₃)₂] | SL-W024-1 | | MeOH | 34.1 | n.d. | 0 |
| D | [Rh(NBD)₂]BF₄ | SL-A241-1 | | MeOH | 55.1 | −37.7 | 46 |
| D | [Rh(NBD)₂]BF₄ | SL-A241-1 | H₂SO₄ | MeOH | 100 | −29.7 | 83.4 |
| D | [Rh(COD)₂]O₃SCF₃ | SL-A241-1 | TFA | 2-Me—THF | 53.5 | −37.2 | 70.1 |
| D | [Rh(NBD)₂]BF₄ | SL-J686-1 | | MeOH | 100 | 79.9 | 74.2 |
| D | [Rh(NBD)₂]BF₄ | SL-J686-1 | H₂SO₄ | MeOH | 96 | 32.8 | 85.6 |
| D | [Rh(COD)₂]O₃SCF₃ | SL-J686-1 | TFA | 2-Me—THF | 17.5 | −8.5 | 18.5 |
| D | [Rh(NBD)₂]BF₄ | SL-F133-1 | | MeOH | 100 | 94 | 70.8 |
| D | [Rh(NBD)₂]BF₄ | SL-F133-1 | H₂SO₄ | MeOH | 22.7 | 53.5 | 61 |
| D | [Rh(COD)₂]O₃SCF₃ | SL-F133-1 | TFA | 2-Me—THF | 18.5 | 76.3 | 24.7 |
| D | [Rh(NBD)₂]BF₄ | SL-M009-1 | | MeOH | 100 | 80 | 71 |
| D | [Rh(NBD)₂]BF₄ | SL-M009-1 | H₂SO₄ | MeOH | 100 | 84.2 | 82.5 |
| D | [Rh(COD)₂]O₃SCF₃ | SL-M009-1 | TFA | 2-Me—THF | 99.1 | 60.4 | 73.5 |
| E | [Rh(NBD)₂]BF₄ | SL-A120-2 | | MeOH | 100 | 36.9 | 76 |
| E | [Rh(NBD)₂]BF₄ | SL-A120-2 | H₂SO₄ | MeOH | 100 | 36.6 | 89.1 |
| E | [Ru(COD)(OOCCF₃)₂] | SL-A120-2 | | MeOH | 100 | 87 | 50 |
| E | [Rh(NBD)₂]BF₄ | SL-J688-1 | | MeOH | 100 | 7.6 | 73.6 |
| E | [Rh(NBD)₂]BF₄ | SL-J688-1 | H₂SO₄ | MeOH | 100 | 7.9 | 86.9 |
| E | [Ru(COD)(OOCCF₃)₂] | SL-J688-1 | | MeOH | 100 | 8.4 | 67 |
| E | [Rh(NBD)₂]BF₄ | SL-C048-1 | | MeOH | 34.4 | −53 | 19.1 |
| E | [Rh(NBD)₂]BF₄ | SL-C048-1 | H₂SO₄ | MeOH | 24 | −68.1 | 50.7 |
| E | [Ru(COD)(OOCCF₃)₂] | SL-C048-1 | | MeOH | 43.5 | n.d. | 1.5 |
| E | [Rh(NBD)₂]BF₄ | SL-T125-1 | | MeOH | 25.3 | −73.3 | 18.9 |
| E | [Rh(NBD)₂]BF₄ | SL-T125-1 | H₂SO₄ | MeOH | <10 | n.d. | n.d. |
| E | [Ru(COD)(OOCCF₃)₂] | SL-T125-1 | | MeOH | 38.3 | n.d. | 2.2 |
| F | [Rh(NBD)₂]BF₄ | SL-A121-1 | | MeOH | 100 | 31.4 | 67 |
| F | [Rh(NBD)₂]BF₄ | SL-A121-1 | H₂SO₄ | MeOH | 100 | 30.5 | 85.4 |
| F | [Rh(COD)₂]O₃SCF₃ | SL-A121-1 | TFA | 2-Me—THF | 94.8 | 39 | 73.6 |
| F | [Rh(NBD)₂]BF₄ | SL-T001-1 | | MeOH | 98.4 | −46.2 | 73.7 |
| F | [Rh(NBD)₂]BF₄ | SL-T001-1 | H₂SO₄ | MeOH | 98.9 | −39.5 | 88.3 |
| F | [Rh(COD)₂]O₃SCF₃ | SL-T001-1 | TFA | 2-Me—THF | 96.9 | −59 | 81.2 |
| F | [Rh(NBD)₂]BF₄ | SL-T121-1 | | MeOH | 100 | 28.8 | 76.5 |
| F | [Rh(NBD)₂]BF₄ | SL-T121-1 | H₂SO₄ | MeOH | 100 | 40.5 | 91.4 |
| F | [Rh(COD)₂]O₃SCF₃ | SL-T121-1 | TFA | 2-Me—THF | 100 | 7.6 | 77 |
| F | [Rh(COD)(SL-P102- | | | MeOH | 38.7 | −40.2 | 23 |
| | 1)]O₃SCF₃ | | | | | | |
| F | [Rh(COD)(SL-P102- | | H₂SO₄ | MeOH | 23.8 | −17.5 | 24.9 |
| | 1)]O₃SCF₃ | | | | | | |
| F | [Rh(COD)(SL-P102- | | | TFE | 43.4 | 2.4 | 26.7 |
| | 1)]O₃SCF₃ | | | | | | |
| G | [Rh(NBD)₂]BF₄ | SL-F356-1 | | MeOH | 100 | 94.5 | 73.3 |
| G | [Rh(NBD)₂]BF₄ | SL-F356-1 | H₂SO₄ | MeOH | 100 | 50.2 | 88.7 |
| G | [Ru(COD)(OOCCF₃)₂] | SL-F356-1 | | MeOH | 43.4 | n.d. | 0.4 |
| G | [Rh(NBD)₂]BF₄ | SL-T002-1 | | MeOH | 100 | −36 | 72.1 |
| G | [Rh(NBD)₂]BF₄ | SL-T002-1 | H₂SO₄ | MeOH | 100 | −38.3 | 84.2 |
| G | [Ru(COD)(OOCCF₃)₂] | SL-T002-1 | | MeOH | 100 | 10.3 | 58.9 |
| G | [Rh(NBD)₂]BF₄ | SL-T129-1 | | MeOH | 100 | 13.2 | 72.6 |
| G | [Rh(NBD)₂]BF₄ | SL-T129-1 | H₂SO₄ | MeOH | 100 | 30.1 | 84.3 |
| G | [Ru(COD)(OOCCF₃)₂] | SL-T129-1 | | MeOH | 75.7 | 73.4 | 27 |
| G | [Rh(COD)(SL-P114- | | | MeOH | 66.5 | −56 | 18.8 |
| | 1)]BF₄ | | | | | | |
| G | [Rh(COD)(SL-P114- | | H₂SO₄ | MeOH | 23.6 | −35 | 10.2 |
| | 1)]BF₄ | | | | | | |
| G | [Rh(COD)(SL-P114- | | | TFE | 32.7 | n.d. | 7.1 |
| | 1)]BF₄ | | | | | | |
| H | [Rh(NBD)₂]BF₄ | SL-F357-1 | | MeOH | 100 | 97.3 | 77.3 |
| H | [Rh(NBD)₂]BF₄ | SL-F357-1 | H₂SO₄ | MeOH | 100 | 14.9 | 88.2 |
| H | [Rh(COD)₂]O₃SCF₃ | SL-F357-1 | TFA | 2-Me—THF | 99.1 | 89.3 | 76.8 |
| H | [Rh(NBD)₂]BF₄ | SL-F102-1 | | MeOH | 100 | −24.2 | 70.3 |
| H | [Rh(NBD)₂]BF₄ | SL-F102-1 | H₂SO₄ | MeOH | 100 | 30.6 | 90.7 |
| H | [Rh(COD)₂]O₃SCF₃ | SL-F102-1 | TFA | 2-Me—THF | 14.6 | n.d. | 5.5 |
| H | [Rh(NBD)₂]BF₄ | SL-W008-1 | | MeOH | 100 | 68.1 | 77.8 |
| H | [Rh(NBD)₂]BF₄ | SL-W008-1 | H₂SO₄ | MeOH | 100 | 68.7 | 93.1 |
| H | [Rh(COD)₂]O₃SCF₃ | SL-W008-1 | TFA | 2-Me—THF | 98.2 | 76.9 | 76.4 |
| H | [Rh(COD)(SL-C040- | | | MeOH | 77.1 | −31.4 | 66.8 |
| | 1)]BF₄ | | | | | | |
| H | [Rh(COD)(SL-C040- | | H₂SO₄ | MeOH | 63.8 | −23.5 | 71.5 |
| | 1)]BF₄ | | | | | | |
| H | [Rh(COD)(SL-C040- | | | TFE | 31.5 | n.d. | 4 |
| | 1)]BF₄ | | | | | | |

The conversion and the diastereoelectivity were determined by HPLC. Uncorrected integrals were used for the calculation of the conversion and the diastereomeric excess (d.e.). Positive d.e. values mean that Compound 3′ is predominately formed, negative d.e.-values indicate preferential formation of Compound 3.

### Example 10

### Ligand Chirality and its Stereochemical Impact on Product

A group of pairs of enantiomeric ligands were tested with some Group VIII metal catalysts to determine the effect of ligand chirality on the diastereoselectivity of the hydrogenation of E-2. Results of this experimental series are summarized in Table 6.

TABLE 6
Asymmetric Hydrogenation of E-2 With Pairs of Enantiomeric Liganded Catalysts

| Entry | | Ligand SL | | | |
|---|---|---|---|---|---|
| No. - | Precursor | # | Solvent | Conv. | 3′:3 |
| 101a | [Rhnbd)₂]BF₄ | SL-F356-2 | MeOH | 100 | 2.1:97.9 |
| 101b | [Rhnbd)₂]BF₄ | SL-F356-1 | MeOH | 100 | 97.2:2.8 |
| 102a | [Ru(cod)(OOCCF₃)₂] | SL-A120-1 | MeOH | 99.1 | 9.1:90.9 |
| 102b | [Ru(cod)(OOCCF₃)₂] | SL-A120-2 | MeOH | 100 | 93.5:6.5 |
| 103a | [Rhnbd)₂]BF₄ | SL-C151-1 | MeOH | 100 | 92.6:7.4 |
| 103b | [Rhnbd)₂]BF₄ | SL-C151-2 | MeOH | 100 | 7.2:92.8 |

Reaction conditions: E-acid: 500 mg (1.37 mmol); s/c: 25; Solvent volume: 17 ml; p(H₂): 10 bar; T: 25° C.; Time 16hrs.

### Experimental Procedure

Exemplary Experiment 101a from Table 6 is described. All manipulations were carried out under an atmosphere of argon. 44.9 mg (0.060 mmol) SL-F356-2 ligand and 20.5 mg (0.055 mmol) [Rh(nbd)₂]BF₄ were placed in a 10 ml Schlenk flask that was previously set under an atmosphere of argon. Then 5 ml degassed methanol was added and this solution and stirred for 15 min at room temperature. Into a second 25 ml Schlenk flask was placed 0.5 g (1.37 mmol) of compound E-2, followed by 12 ml MeOH. The clear solution was stirred for 5 min, and subsequently, the substrate and the catalyst solution were each transferred via canula into a 50 ml stainless steel reactor that was previously set under an atmosphere of argon. The reactor was sealed, purged with argon in three cycles (1 bar/20 bar) and thereafter, the argon replaced by hydrogen (4 cycles 1 bar/20 bar). The reactor pressure was set to 10 bar hydrogen and stirring started. After 16 hrs reaction time, the pressure was released. Upon derivatization with TMS diazomethane, the crude product was analyzed with respect to conversion, chemoselectivity and diastereomeric ratio using the HPLC method described in the Appendix. The conversion was >99.8%, the product formation quantitative and the diastereomeric purity of hydrogenated acid product was 97.9% (Compound 3).

### Example 11

### Typical Procedure for the Asymmetric Hydrogenation of E-2

The following procedure is typical for all hydrogenation experiments. All manipulations were carried out under an atmosphere of argon.
1.34 mg (0.022 mmol) SL-J504-1 ((R)—(S)-cy₂PF—P(oTol)₂) and 0.83 mg (0.020 mmol) [Rh(cod)₂]BF₄ are placed in a 10 ml Schlenk flask that was previously set under an atmosphere of argon. Then 1 ml degassed methanol was added and this solution stirred for 10 min. In a second Schlenk flask, a solution of 1.5 g E-2 (SOL03293-8; 4.1 mmol) in 19 ml degassed methanol was prepared and stirred for 15 min, and subsequently 110 μl (0.5 eq/eq E-2) sulphuric acid was added to this solution. Both, the substrate and the catalyst solution were transferred via cannula into a 50 ml stainless steel autoclave. The reactor was sealed, purged with argon (setting the pressure to 10-12 bar and releasing it; four times repeated) and finally, the argon replaced by hydrogen (4 cycles 20 bar/1 bar). The reactor was heated to 40° C., the pressure set to 20 bar, and stirring started. After 16 hrs reaction time, the pressure was released. The reaction mixture was a clear solution.

A portion of the crude reaction mixture (0.5 ml) was dissolved with 0.5 ml methanol. An approximate ten-fold excess of TMS-diazomethane (2M in Et₂O) was added and the mixture stirred for 30 min. Then, the solvent and the remaining TMS-diazomethane were removed to dryness under reduced pressure. This crude product was analyzed with respect to conversion, chemoselectivity and diastereoselectivity using the HPLC method described herein as well as by LC/MS (Electrospray Ionization). The conversion was 98% and the diastereomeric ratio (αS,3R,4R) isomer Compound 4):(αR,3R,4R) isomer Compound 4′ (94.7:5.3).

### Example 12

### Alternate Typical Hydrogenation Procedure

The equipment included a 16 L-Inconel reactor equipped with: hollow shaft stirrer, sampling tube, internal cooling coils, electric heating, pre-conditioned through a dilute sacrifice run; 1 L-Schlenk flask; 10 L glass vessel; and 10 L glass reactor.

The reactor was charged with solid E-2 through a 4 cm diameter tube. 5 L MeOH was added through the 4 cm diameter tube and the mixture was slowly stirred. A mixture of H₂SO₄ in 0.5 L MeOH was added. The reactor was closed and stirred for 30 min at rt. The reactor was degassed through pressurizing with nitrogen (10 bar, under stirring) and subsequent depressurization (5 times). A gentle vacuum (50 mbar) was applied on the reactor. Chiral phosine ligand SL-J504-1 and [Rh(NBD)2]BF4 were placed in a 1 L Schlenk bulb (equipped with a rubber septum) and set under an atmosphere of argon. The Schlenk bulb was charged with dried and degassed MeOH (700 mL) and the mixture was stirred for 30 min at rt. The rubber septum of the Schlenk bulb (attached to an argon line) was punctured with a thin tube attached to the reactor and the catalyst solution was sucked in. The reactor was degassed through pressurizing with nitrogen (10 bar, under stirring) and subsequent depressurization (3 times). The reactor was set under hydrogen through pressurizing with hydrogen (8 bar, under stirring) and subsequent depressurization (3 times). The stirrer was set to 1000 rpm and the mixture was heated to 60° C. under a constant pressure of 8 bar. After 7 hours, a sample was removed through the sampling tube. After 7 hours, the mixture was heated to 80° C. After an additional 15 hours at 80° C., a sample was removed through the sampling tube. After a total reaction time of 22 hours the heater was switched off. After reaching rt, the reactor was set under nitrogen through pressurizing with nitrogen (10 bar, under stirring) and subsequent depressurization (3 times). The mixture was pumped out of the reactor into a 10 L glass vessel through the sampling tube applying a gentle nitrogen pressure. The reactor was rinsed with 0.5 L MeOH, and the wash solution was added to the 10 L glass vessel. A 10 L-glass reactor was equipped with addition funnel, nitrogen inlet, reflux condenser. The reactor was set under inert atmosphere. The solution from the hydrogenation was transferred into the 10 L-glass reactor. H₂SO₄ (173 mL, 318 g, 3.24 mol, 1.0 eq) was added drop wise via an addition funnel. The temperature rose to 30° C. The mixture was heated to 65° C. during 30 minutes and maintained at 65° C. for 22 hours with stirring. The heating source was removed and reaction was cooled to rt over 3 hours. The reactor was cooled with an ice-bath. 100 mg of seeding crystals were added. By the time the mixture reached a temperature of approx. 15° C., crystallisation had started. The ice bath was replaced with a cool bath that was allowed to warm slowly to rt (overnight). The mixture was cooled again to 5° C. (ice-bath) and stirred for 30min at 5° C., and filtered over a suction filter equipped with a cotton filter. The solid was rinsed with cold MeOH (1.0 L) and dried at 40° C./20 mbar for 72 hours to provide the product.
1^{st} crop: 1.26 kg, 80% yield, 96% 4 (4/4′>98:2), 1% E-ester, 2% Z-ester, 1% 3; approx 50% methyl sulfate salt.
Mother liquor was concentrated to approx 1-2 L (45° C., 200 mbar) and cooled to rt. 2nd crop: 0.12 kg, 7% yield, 92% 4 (4/4′ 96:4), 3% E-ester, 1% Z-ester, 4% 3.

Analytical Methods

Conversion and the diastereoselectivity were determined as follows. The crude reaction mixture was reacted with an excess of TMS-diazomethane and the completeness of the reaction was checked by LC/MS. The excess TMS-diazomethane and the solvent were stripped off, and the crude residue dissolved in i-propanol. The most relevant parameters of this HPLC method are as indicated below.

### Determination of Conversion and Chemoselectivity for the Hydrogenation

### ColumnDaicel CHIRALCEL OD H(4.6*250 mm)

| | |
|---|---|
| Solvents | iPrOH (8), hexane (92), diethyl amine DEA (0.5) |
| | isocratic |
| Flow | 1.0 ml/min. |
| Detection: | 273 nm |
| Temperature | 40° C. |
| Sample concentration | 1.0 mg/ml |
| Injection volume | 0.5 *L |
| Run time | 50 min. |

### Retention Times:

| | |
|---|---|
| Z-2 (as methyl ester) | 14.2 min (26.2 min @ flow 0.5 ml/min) |
| E-2 (as methyl ester) | 15.3 min (27.8 min @ flow 0.5 ml/min) |
| (αR,3R,4R)-4′ | 16.6 min (29.3 min @ flow 0.5 ml/min) |
| (αS,3R,4R)-4 | 18.2 min (32.5 min @ flow 0.5 ml/min) |

Table of Chiral Phosphine Ligands

| | |
|---|---|
| 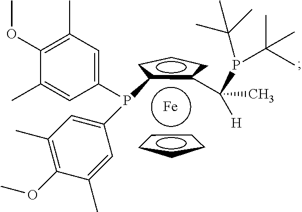 | SL-J013-1 |
| 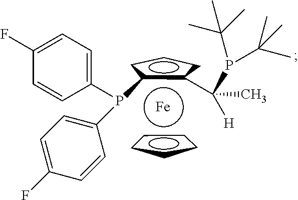 | SL-J014-1 |
| 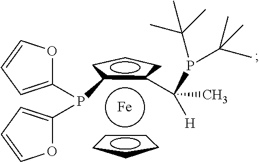 | SL-J212-1 |
| 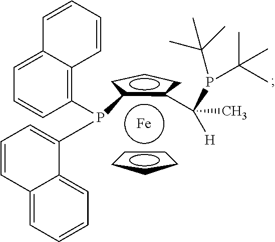 | SL-J216-1 |
| 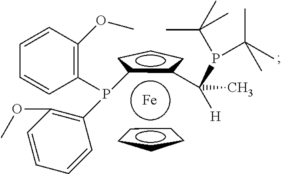 | SL-J219-1 |
| 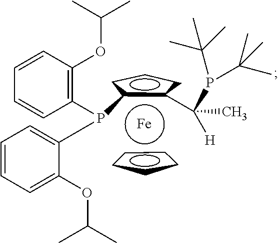 | SL-J226-1 |
| 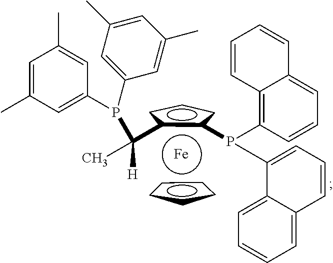 | SL-J404-2 |
| 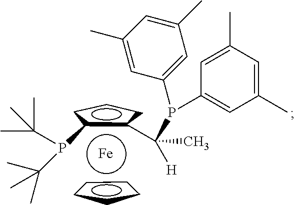 | SL-J501-1 |
| 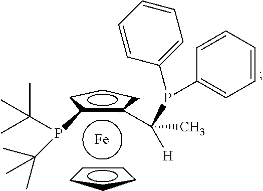 | SL-J502-1 |
| 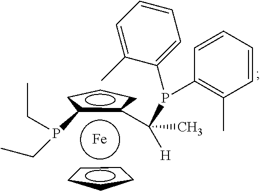 | SL-J503-1 |
| 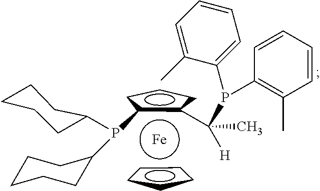 | SL-J504-1 |
| 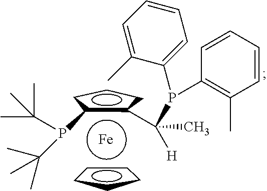 | SL-J505-1 |
| 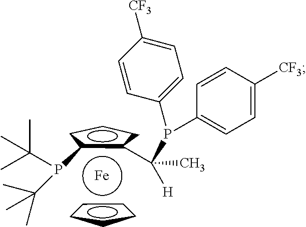 | SL-J506-1 |
| 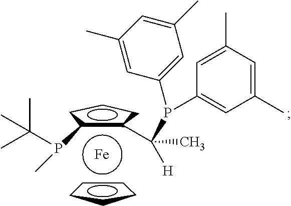 | SL-J510-1 |
| 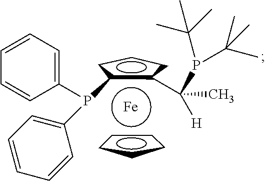 | SL-J002-1 |
| 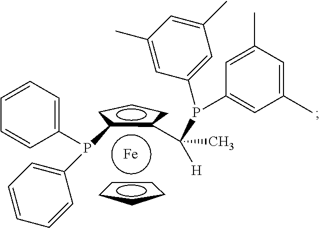 | SL-J005-1 |
| 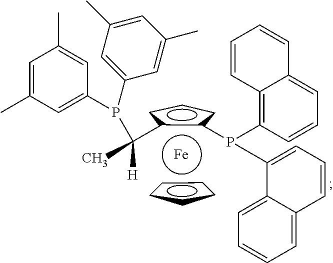 | SL-W001-2 |
| 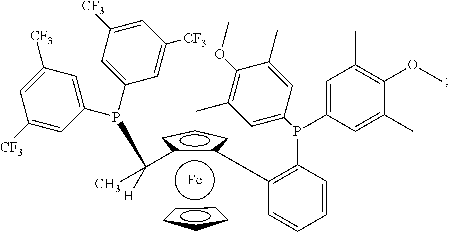 | SL-W005-2 |
| 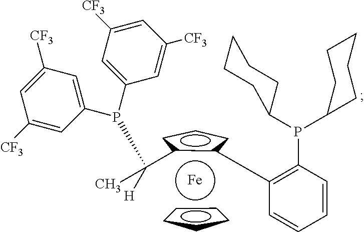 | SL-W008-2 |
| 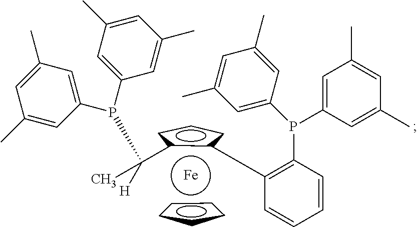 | SL-W009-2 |
| 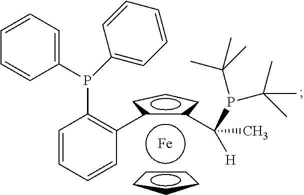 | SL-W012-1 |
| 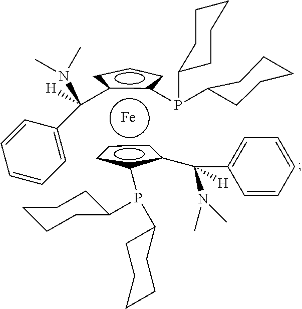 | SL-M002-2 |
| 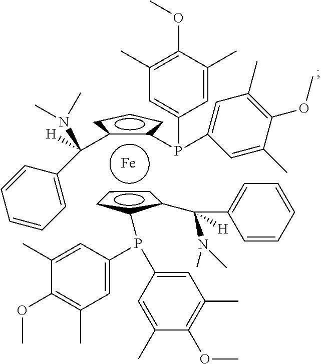 | SL-M004-2 |
| 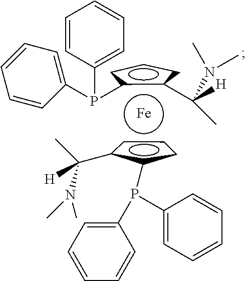 | SL-M031-2 |
| 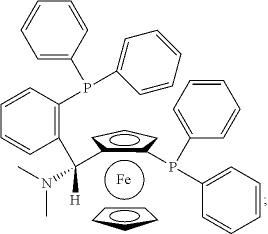 | SL-T001-2 |
| 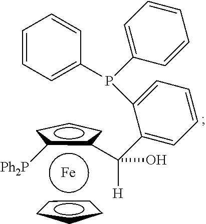 | SL-T021-2 |
| 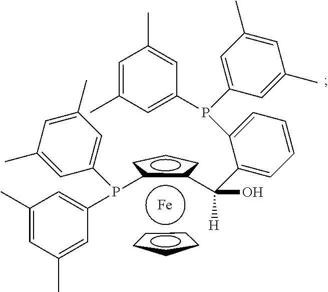 | SL-T025-2 |
| 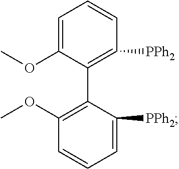 | SL-A101-2 |
| 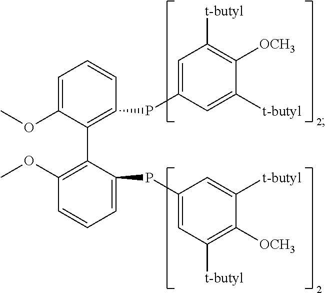 | SL-A109-2 |
| 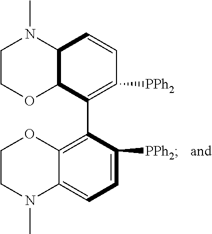 | SL-A001-2 |
| 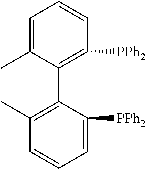 | SL-A131-2 |
| 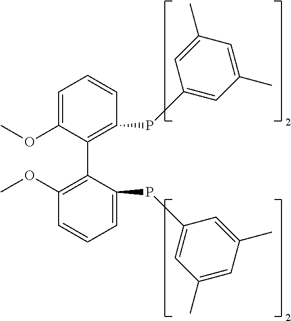 | SL-A120-2 |
| 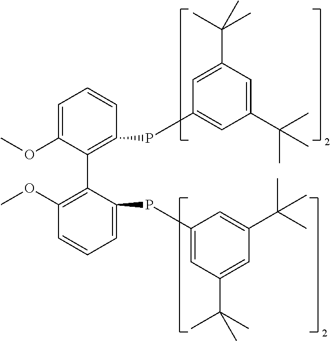 | SL-A121-1 |
| 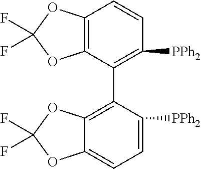 | SL-A153-1 |
| 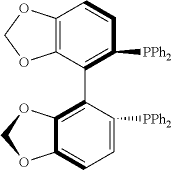 | SL-A241-1 |
| 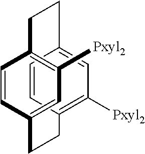 | SL-C020-2 |
| 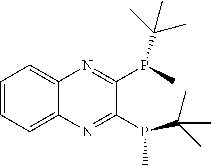 | SL-C048-1 |
| 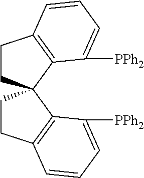 | SL-C151-1 |
| 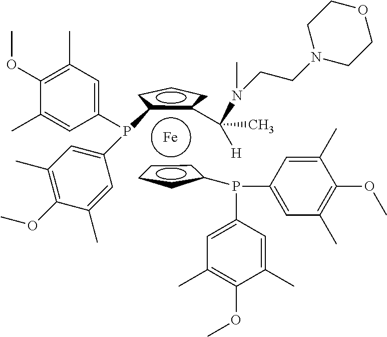 | SL-F124-1 |
| 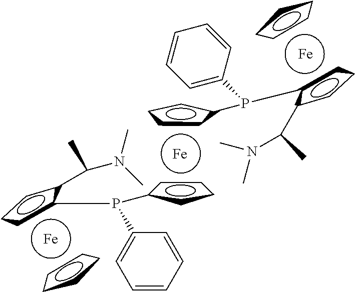 | SL-F131-1 |
| 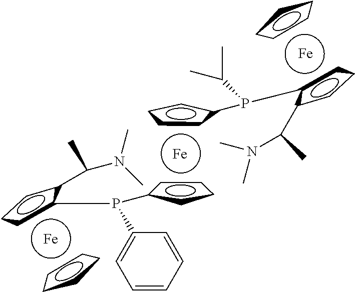 | SL-F132-1 |
| 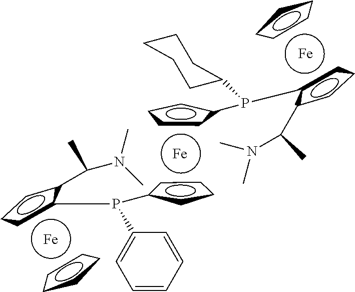 | SL-F133-1 |
| 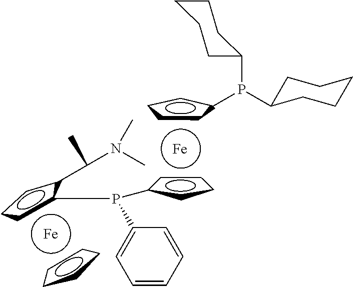 | SL-F356-1 |
| 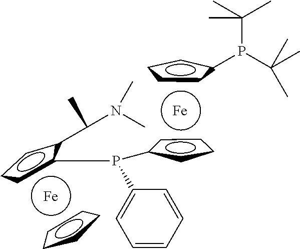 | SL-357-1 |
| 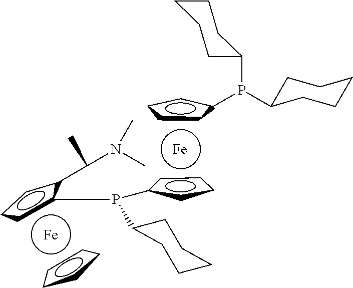 | SL-F365-1 |
| 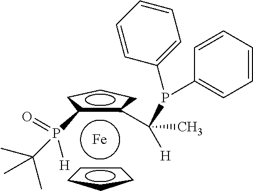 | SL-J681-1 |
| 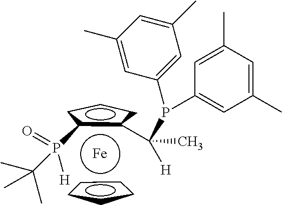 | SL-J683-1 |
| 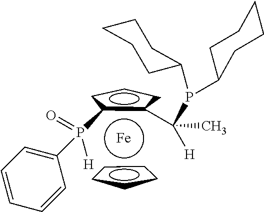 | SL-J686-1 |
| 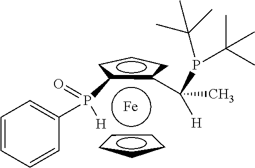 | SL-J688-1 |
| 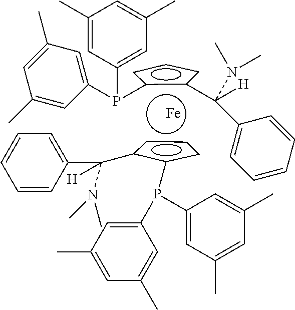 | SL-M009-1 |
| 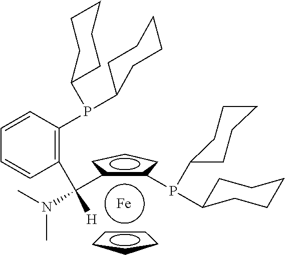 | SL-T002-1 |
| 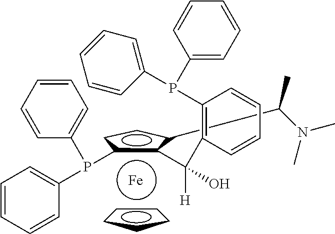 | SL-T121-1 |
| 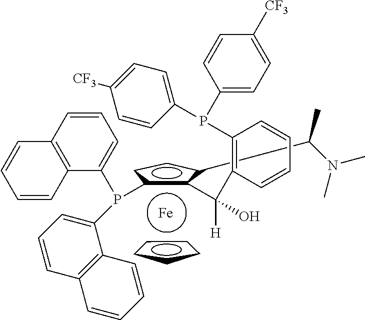 | SL-T125-1 |
| 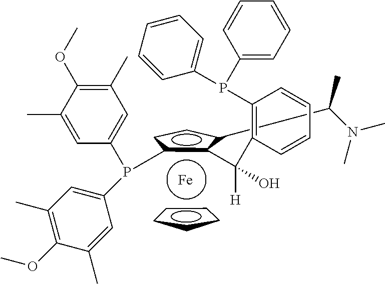 | SLT129-1 |
| 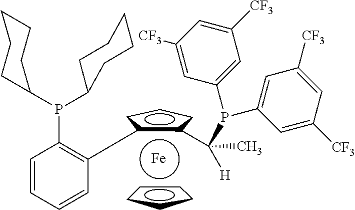 | SLW008-1 |
| 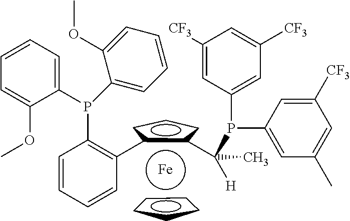 | SL-W024-1 |
| 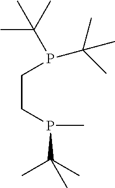 [SMILES: C[P@](CCP(C(C)(C)C)C(C)(C)C)C(C)(C)C] | SL-C040-1 |
| 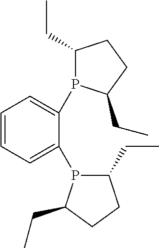 [SMILES: CC[C@@H]1CC[C@@H](CC)P1c1ccccc1P1[C@H](CC)CC[C@H]1CC] | SL-P102-1 |
| 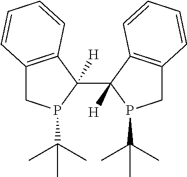 [SMILES: CC(C)(C)[P@]1Cc2ccccc2[C@H]1[C@@H]1c2ccccc2C[P@@]1C(C)(C)C] | SL-P114-1 |
| 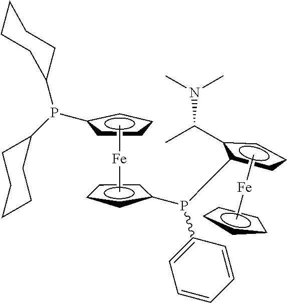 | SL-F356-2 |
| 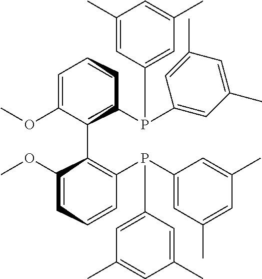 | SL-A120-1 |
| 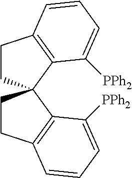 | SL-C151-2 |

The disclosures of each patent, patent application and publication cited or described in this document are hereby incorporated herein by reference, in their entirety.

Various modification of the invention, in addition to those described herein, will be apparent to those skilled in the art from the foregoing description. Such modifications are also intended to fall within the scope of the appended claims.

The invention illustratively disclosed herein suitably may be practiced in the absence of any element which is not specifically disclosed herein. The invention illustratively disclosed herein suitably may also be practiced in the absence of any element which is not specifically disclosed herein and that does not materially affect the basic and novel characteristics of the claimed invention.

All processes disclosed in association with the present invention are contemplated to be practiced on any scale, including milligram, gram, multigram, kilogram, multikilogram or commercial industrial scale.

The invention can be illustrated by the following embodiments enumerated in the numbered paragraphs below:
[Embodiment 1] A process for preparing an N-alkylpiperidine compound of Formula Ia or Formula Ib, or mixture thereof:

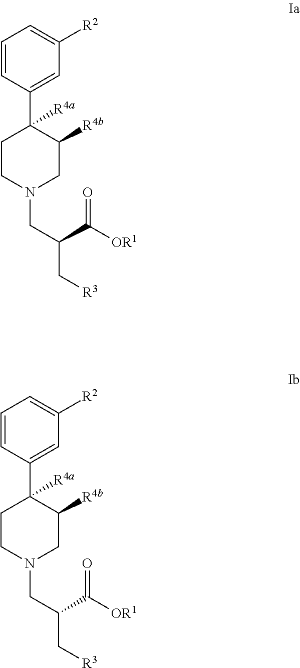

wherein:
each R¹ is independently H, alkyl, or aralkyl;
each R² is independently Cl, Br, I, —C(═O)OR^{5b}, —CN, —OR⁶, or —CONR⁷R⁸;
each R³ is independently H, alkyl, cycloalkyl, or aryl;
each R^{4a} and R^{4b} is independently C₁₋₆alkyl;
each R^{5b} is independently H or alkyl;
each R⁶ is independently H, alkyl, cycloalkyl, alkylcycloalkyl, aralkyl, or an hydroxyl protecting group;
each R⁷ is independently H, alkyl, cycloalkyl, alkylcycloalkyl, or aralkyl; and
each R⁸ is independently H, alkyl, aralkyl, or aryl;
or a salt thereof;
   comprising contacting an N-alkenylpiperidine compound of Formula IIa, Formula IIb, Formula IIc or Formula IId, or mixture thereof:

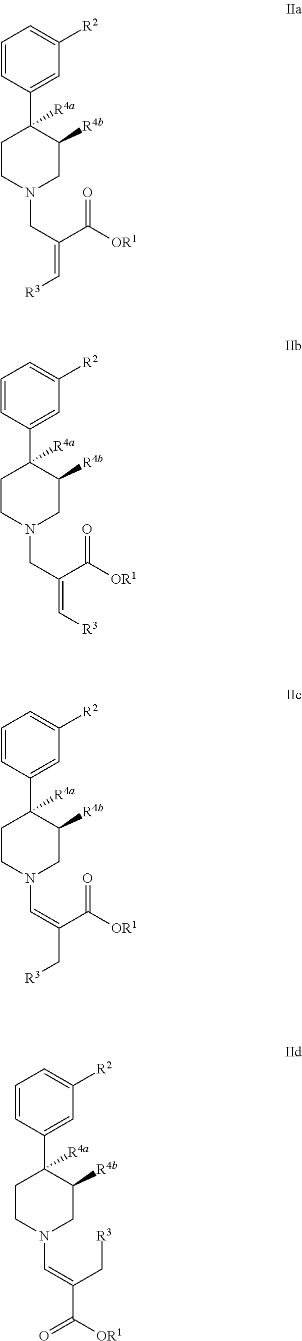

with hydrogen in the presence of a hydrogenation catalyst and a chiral phosphorus-containing ligand for a time and under conditions effective to provide the compound of Formula Ia, the compound of Formula Ib, or mixture thereof.
[Embodiment 2] A process of embodiment 1, wherein the N-alkenylpiperidine compound of Formula IIa or The invention can be illustrated by the following embodiments enumerated in the numbered paragraphs below: Formula IIb or mixture thereof is prepared by a process comprising contacting a piperidine compound of Formula III:

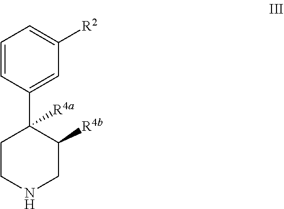

with an alkene compound of Formula IVa or Formula IVb or mixture thereof:

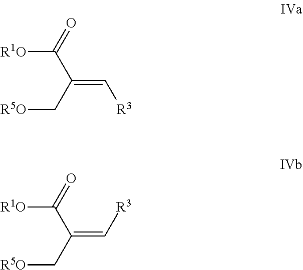

wherein:
each R⁵ is independently alkyl, aralkyl, or —C(═O)R^{5a}; and
each R^{5a} is independently H, alkyl, or aralkyl;
   for a time and under conditions effective to provide the N-alkenylpiperidine compound of Formula IIa or Formula II, or mixture thereof.
[Embodiment 3] A process of embodiment 1, wherein the N-alkylpiperidine compounds of Formula Ia and Formula Ib are prepared in a molar ratio of greater than about 1 to about 1.
[Embodiment 4] A process of embodiment 3, wherein the N-alkylpiperidine compounds of Formula Ia and Formula Ib are prepared in a molar ratio of at least about 2 to about 1.
[Embodiment 5] A process of embodiment 4, wherein the N-alkylpiperidine compounds of Formula Ia and Formula Ib are prepared in a molar ratio of at least about 4 to about 1.
[Embodiment 6] A process of embodiment 5, wherein the N-alkylpiperidine compounds of Formula Ia and Formula Ib are prepared in a molar ratio of at least about 9 to about 1.
[Embodiment 7] A process of embodiment 6, wherein the N-alkylpiperidine compounds of Formula Ia and Formula Ib are prepared in a molar ratio of at least about 19 to about 1.
[Embodiment 8] A process of embodiment 1, wherein each R¹ is independently H or C₁₋₆alkyl.
[Embodiment 9] A process of embodiment 8, wherein each R¹ is independently H or CH₃.
[Embodiment 10] A process of embodiment 9, wherein R¹ is H.
[Embodiment 11] A process of embodiment 1, wherein R² is —OR⁶.
[Embodiment 12] A process of embodiment 1, wherein each R³ is independently alkyl, cycloalkyl, or aryl.
[Embodiment 13] A process of embodiment 12, wherein each R³ is independently cycloalkyl or aryl.
[Embodiment 14] A process of embodiment 13, wherein R³ is optionally substituted cyclohexyl.
[Embodiment 15] A process of embodiment 13, wherein R³ is aryl.
[Embodiment 16] A process of embodiment 15, wherein R³ is optionally substituted phenyl.
[Embodiment 17] A process of embodiment 1, wherein R^{4a} and R^{4b} are each independently C₁₋₃alkyl.
[Embodiment 18] A process of embodiment 17, wherein R^{4a} and R^{4b} are methyl.
[Embodiment 19] A process of embodiment 11, wherein each R⁶ is independently H, alkyl, aralkyl, or an hydroxyl protecting group.
[Embodiment 20] A process of embodiment 19, wherein each R⁶ is independently H, aralkyl, or an hydroxyl protecting group.
[Embodiment 21] A process of embodiment 20, wherein each R⁶ is independently H or an hydroxyl protecting group.
[Embodiment 22] A process of embodiment 21, wherein R⁶ is H.
[Embodiment 23] A process of embodiment 1, wherein at least one of R⁷ and R⁸ is H.
[Embodiment 24] A process of embodiment 23, wherein R⁷ and R⁸ are H.
[Embodiment 25] A process of embodiment 4, wherein each R¹ is independently H or C₁₋₆alkyl, R² is —OH, R³ is phenyl, and R^{4a} and R^{4b} are methyl.
[Embodiment 26] A process of embodiment 2, wherein R⁵ is —C(═O)R^{5a}.
[Embodiment 27] A process of embodiment 26, wherein each R^{5a} is independently alkyl.
[Embodiment 28] A process of embodiment 1, wherein the N-alkenylpiperidine compound is an N-alkenylpiperidine compound of Formula IIa or Formula IIb, or mixture thereof.
[Embodiment 29] A process of embodiment 28, wherein the N-alkenylpiperidine compound is an N-alkenylpiperidine compound of Formula IIa.
[Embodiment 30] A process of embodiment 28, wherein the N-alkenylpiperidine compound comprises a mixture of N-alkenylpiperidine compounds of Formula IIa and Formula IIb.
[Embodiment 31] A process of embodiment 1, wherein the contacting is carried out at a temperature of from about 10° C. to about 100° C.
[Embodiment 32] A process of embodiment 31, wherein the temperature is from about 20° C. to about 85° C.
[Embodiment 33] A process of embodiment 32, wherein the temperature is from about 20° C. to about 65° C.
[Embodiment 34] A process of embodiment 1, wherein the contacting is carried out in a reactor into which hydrogen gas is charged at a pressure of from about 1 bar to about 150 bar.
[Embodiment 35] A process of embodiment 34, wherein hydrogen gas is charged into the reactor at a pressure of from about 1 bar to about 80 bar.
[Embodiment 36] A process of embodiment 35, wherein hydrogen gas is charged into the reactor at a pressure of from about 3 bar to about 50 bar.
[Embodiment 37] A process of embodiment 36, wherein hydrogen gas is charged into the reactor at a pressure of from about 3 bar to about 30 bar.
[Embodiment 38] A process of embodiment 1, wherein the molar ratio of the N-alkenylpiperidine compound to the hydrogenation catalyst is from about 10 to about 50,000.
[Embodiment 39] A process of embodiment 38, wherein the molar ratio of the N-alkenylpiperidine compound to the hydrogenation catalyst is from about 100 to about 10,000.
[Embodiment 40] A process of embodiment 39, wherein the molar ratio of the N-alkenylpiperidine compound to the hydrogenation catalyst is from about 100 to about 2,000.
[Embodiment 41] A process of embodiment 1, wherein the contacting time is from about 1 to about 100 hours.
[Embodiment 42] A process of embodiment 41, wherein the contacting time is from about 1 to about 24 hours.
[Embodiment 43] A process of embodiment 42, wherein the contacting time is from about 1 to about 10 hours.
[Embodiment 44] A process of embodiment 1, wherein the hydrogenation catalyst is heterogeneous.
[Embodiment 45] A process of embodiment 44, wherein the heterogeneous hydrogenation catalyst comprises palladium.
[Embodiment 46] A process of embodiment 1, wherein the hydrogenation catalyst is homogeneous.
[Embodiment 47] A process of embodiment 46, wherein the homogeneous hydrogenation catalyst comprises a Group VIII transition metal.
[Embodiment 48] A process of embodiment 47, wherein the Group VIII transition metal catalyst comprises rhodium, ruthenium, or iridium.
[Embodiment 49] A process of embodiment 48, wherein the Group VIII transition metal catalyst comprises rhodium.
[Embodiment 50] A process of embodiment 1, wherein the chiral phosphorus-containing ligand is a chiral tertiary diphosphine.
[Embodiment 51] A process of embodiment 50, wherein the chiral tertiary diphosphine or mixture thereof is selected from the group consisting of:

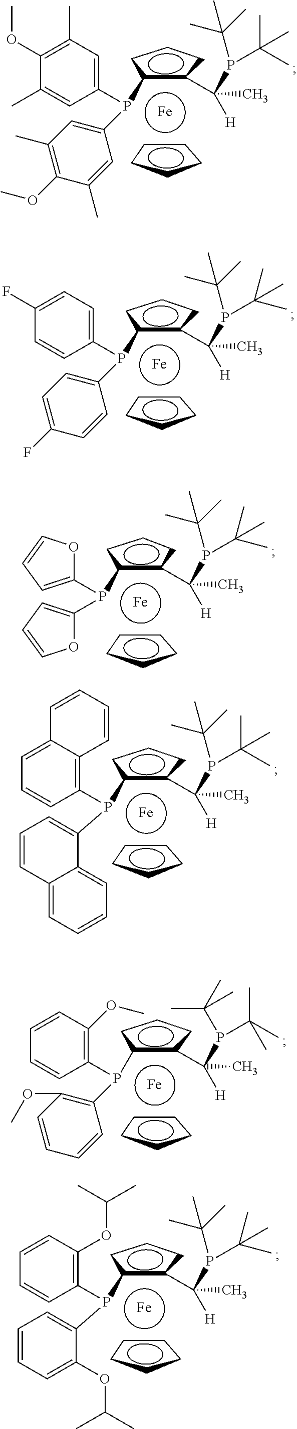

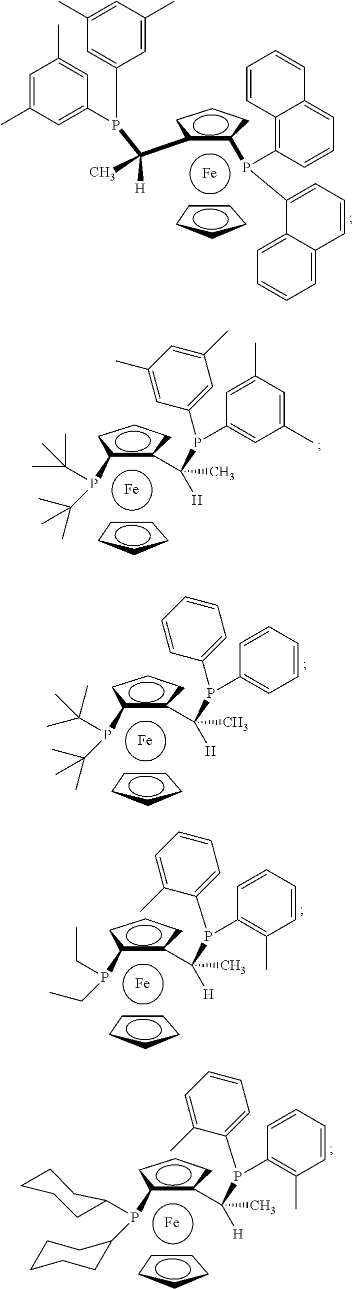

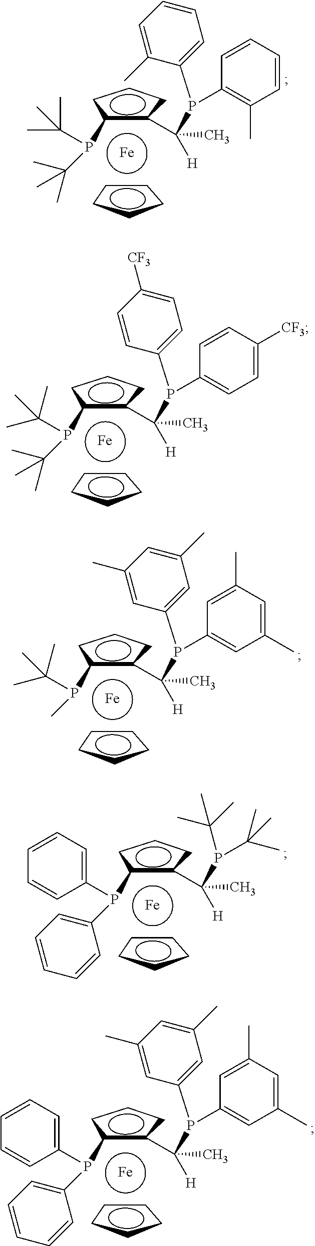

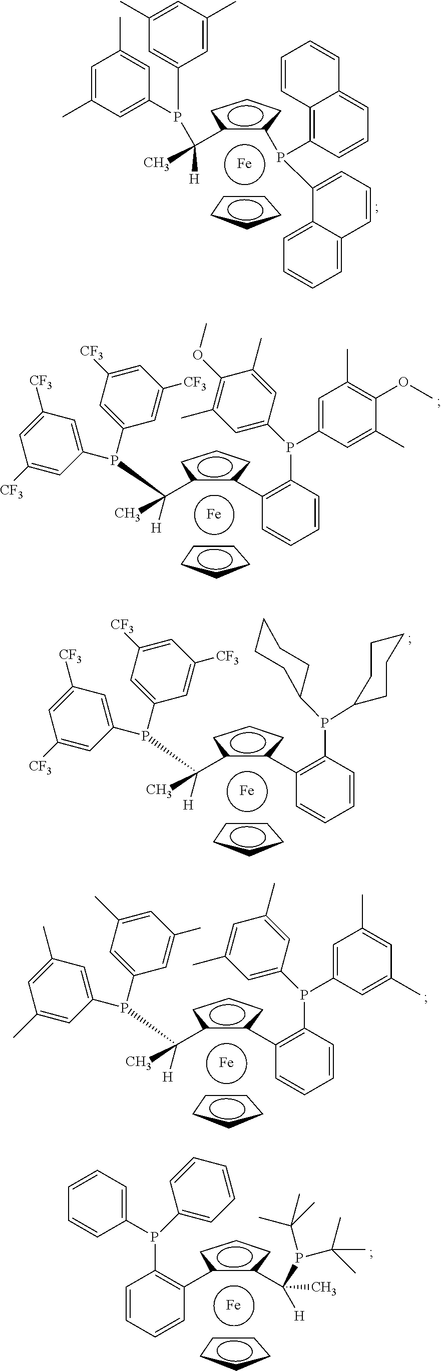

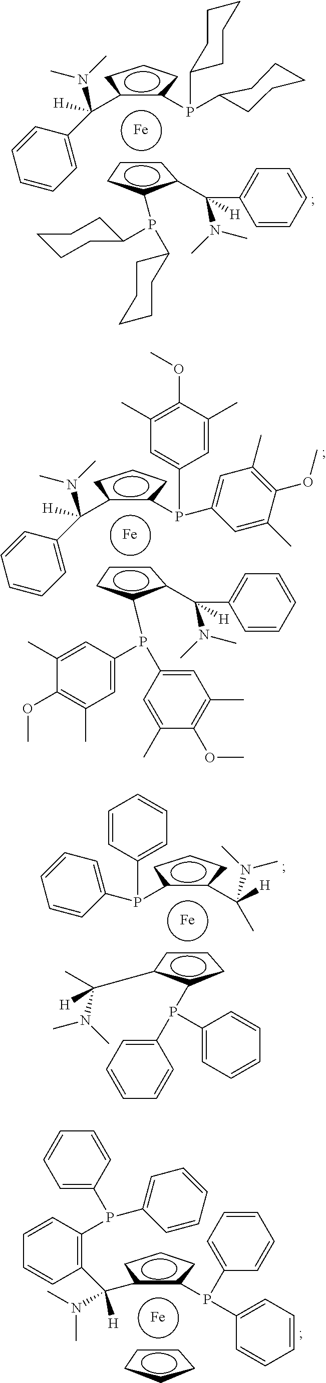

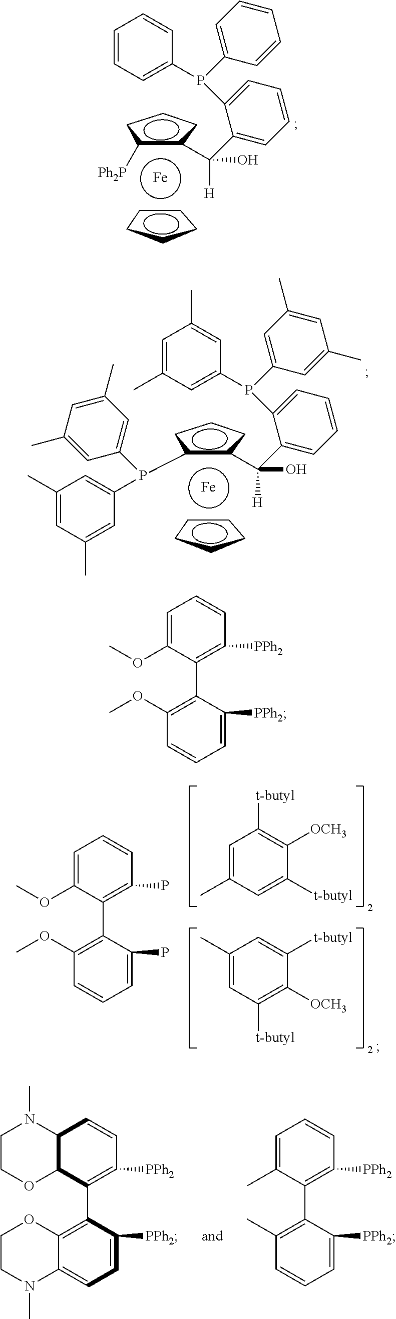

or a mixture thereof.
[Embodiment 52] A process of embodiment 51, wherein the chiral tertiary diphosphine or mixture thereof is selected from the group consisting of:

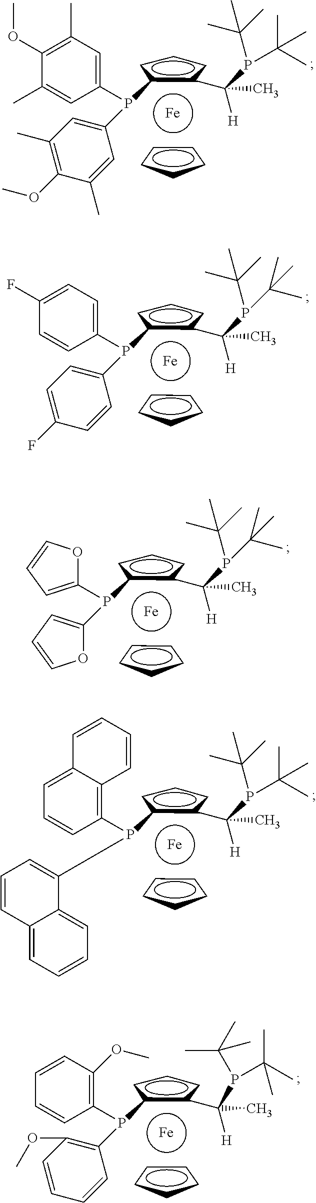

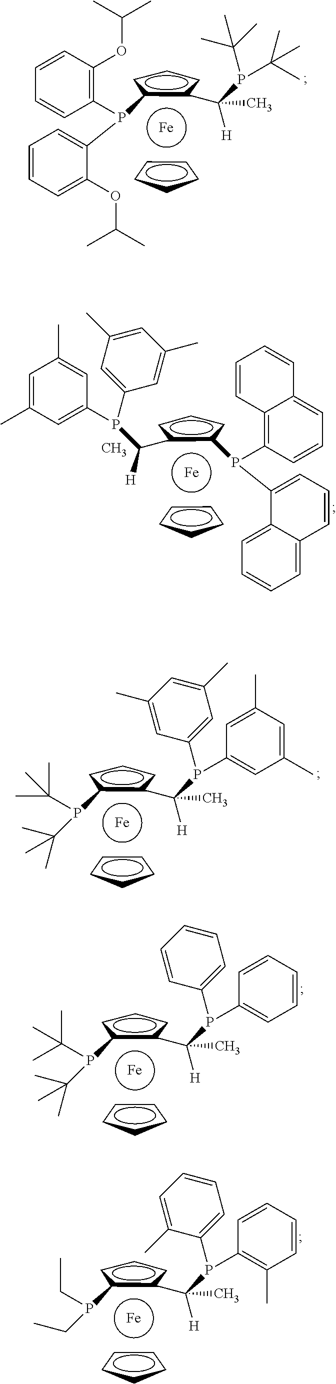

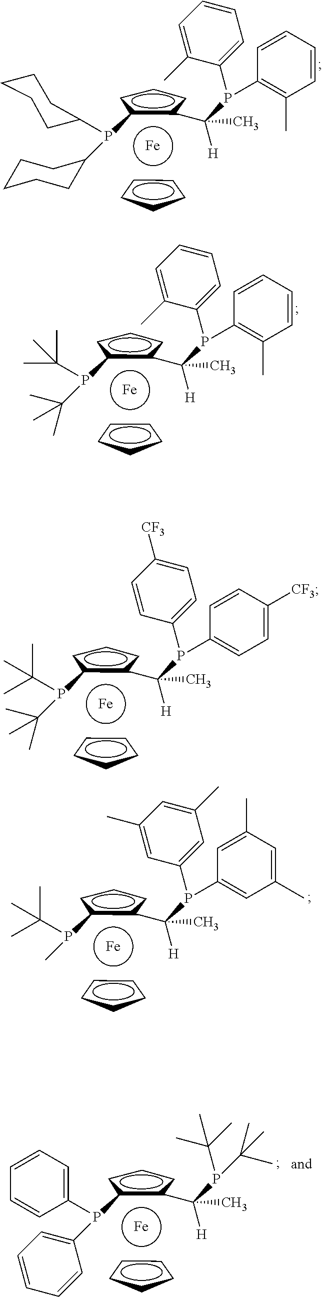

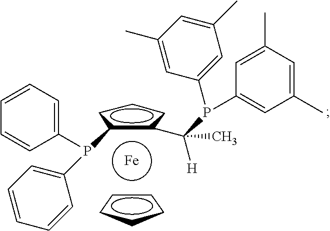

or a mixture thereof.
[Embodiment 53] A process of embodiment 51, wherein the chiral tertiary diphosphine or mixture thereof is selected from the group consisting of:

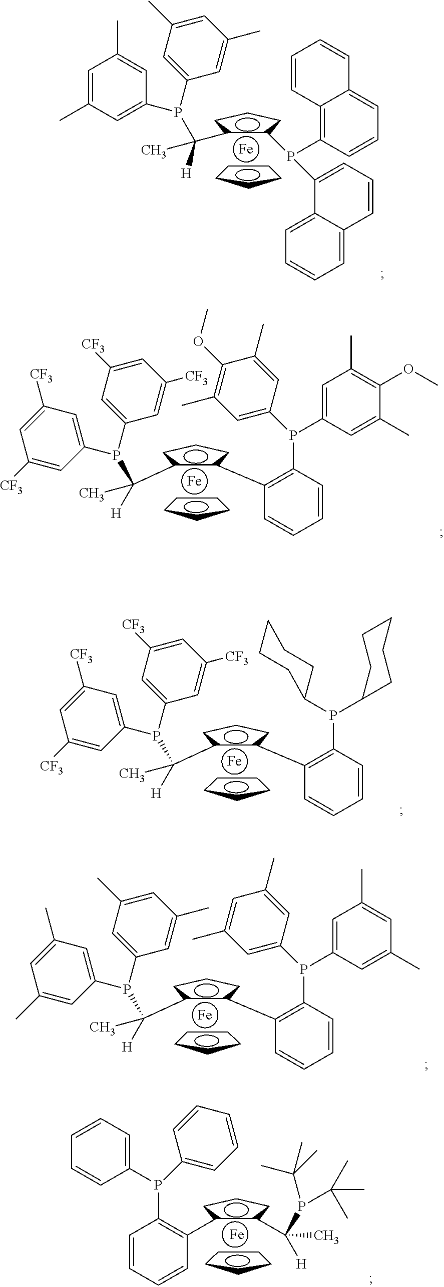

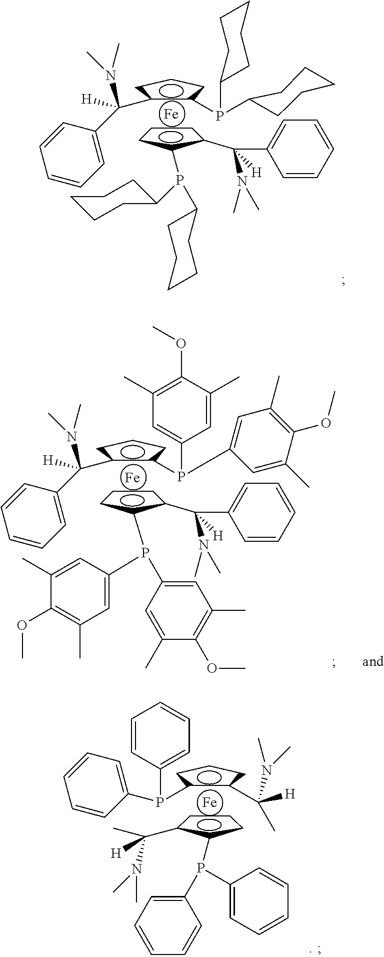

or a mixture thereof.
[Embodiment 54] A process of embodiment 51, wherein the chiral tertiary diphosphine or mixture thereof is selected from the group consisting of:

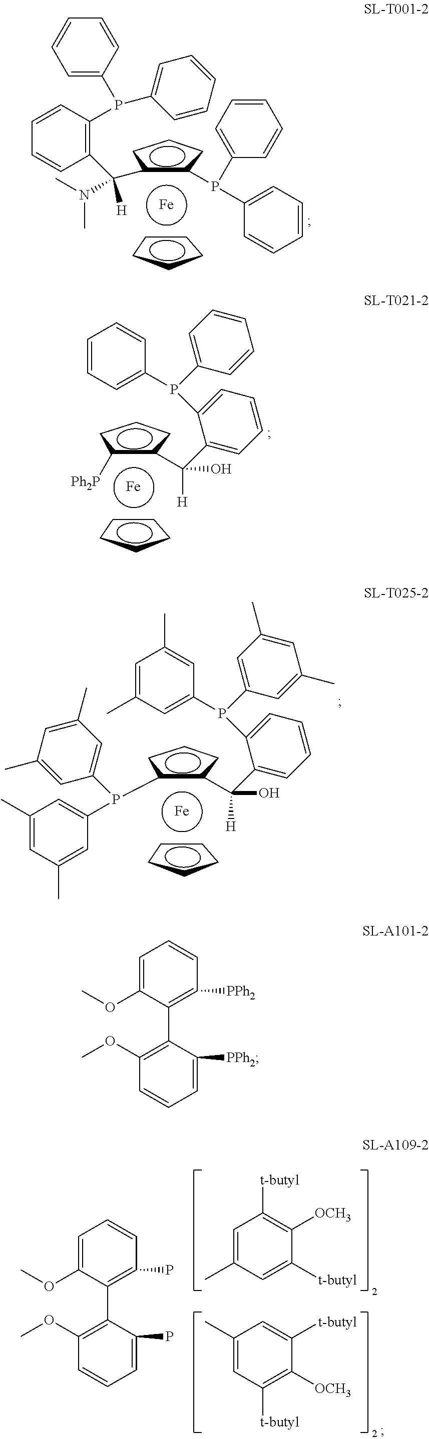

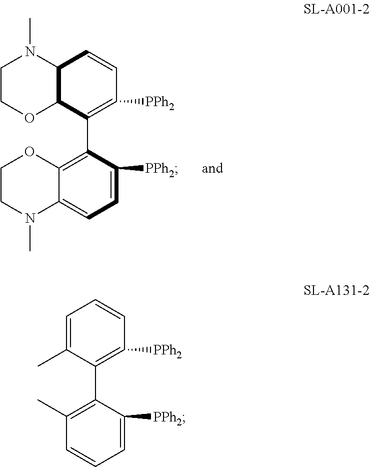

or a mixture thereof.
[Embodiment 55] A process of embodiment 1, wherein the contacting is carried out in a solvent.
[Embodiment 56] A process of embodiment 55, wherein the solvent comprises an alcoholic solvent, an ether, an aromatic hydrocarbon, a chlorinated hydrocarbon, an ester, a lactone, or mixture thereof.
[Embodiment 57] A process of embodiment 56, wherein the solvent comprises an ether.
[Embodiment 58] A process of embodiment 57, wherein the solvent comprises tetrahydrofuran or dioxane or a mixture thereof.
[Embodiment 59] A process of embodiment 56, wherein the solvent comprises an alcoholic solvent.
[Embodiment 60] A process of embodiment 59, wherein the solvent further comprises water.
[Embodiment 61] A process of embodiment 59, wherein the alcoholic solvent comprises a C₁₋₃alkanol.
[Embodiment 62] A process of embodiment 61, wherein the alcoholic solvent comprises methanol.
[Embodiment 63] A process of embodiment 1, wherein the contacting is carried out in the presence of an additive.
[Embodiment 64] A process of embodiment 63, wherein the additive is a proton acid or an amine.
[Embodiment 65] A process of embodiment 64, wherein the additive is an amine.
[Embodiment 66] A process of embodiment 65, wherein the amine is N,N,N,N-tetramethylguanidine.
[Embodiment 67] A process of embodiment 64, wherein the additive is a proton acid.
[Embodiment 68] A process of embodiment 67, wherein the proton acid is selected from the group consisting of an alkylsulfonic acid, an arylsulfonic acid, sulfuric acid, hydrochloric acid, and a carboxylic acid.
[Embodiment 69] A process of embodiment 68, wherein the proton acid is selected from the group consisting of an alkylsulfonic acid and an arylsulfonic acid.
[Embodiment 70] A process of embodiment 69, wherein the proton acid is methanesulfonic acid.
[Embodiment 71] A process for preparing an N-alkenylpiperidine compound of Formula IIa or Formula IIb, or mixture thereof:

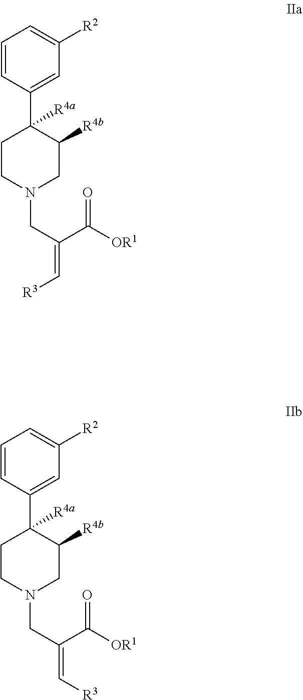

wherein:
each R¹ is independently H, alkyl, or aralkyl;
each R² is independently Cl, Br, I, —C(═O)OR^{5b}, —CN, —OR⁶, or —CONR⁷R⁸;
each R³ is independently H, alkyl, cycloalkyl, or aryl;
each R^{4a} and R^{4b} is independently C₁₋₆alkyl;
each R^{5b} is independently H or alkyl;
each R⁶ is independently H, alkyl, cycloalkyl, alkyl cycloalkyl, aralkyl, or an hydroxyl protecting group;
each R⁷ is independently H, alkyl, cycloalkyl, alkylcycloalkyl, or aralkyl; and
each R⁸ is independently H, alkyl, aralkyl, or aryl;
or a salt thereof;
   comprising contacting a piperidine compound of Formula III:

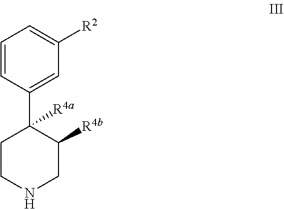

with an alkene compound of Formula IVa or Formula IVb, or mixture thereof:

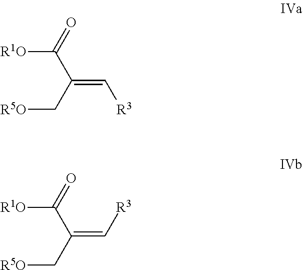

wherein:
each R⁵ is independently alkyl, aralkyl, or —C(═O)R^{5a}; and
each R^{5a} is independently H, alkyl, or aralkyl;
   for a time and under conditions effective to provide the N-alkenylpiperidine compound of Formula IIa, Formula II, or mixture thereof.
[Embodiment 72] A process of embodiment 71, wherein each R¹ is independently H or C₁₋₆alkyl.
[Embodiment 73] A process of embodiment 72, wherein each R¹ is independently H or CH₃.
[Embodiment 74] A process of embodiment 73, wherein R¹ is H.
[Embodiment 75] A process of embodiment 71, wherein R² is —OR⁶.
[Embodiment 76] A process of embodiment 71, wherein each R³ is independently alkyl, cycloalkyl, or aryl.
[Embodiment 77] A process of embodiment 76, wherein each R³ is independently cycloalkyl or aryl.
[Embodiment 78] A process of embodiment 77, wherein R³ is optionally substituted cyclohexyl.
[Embodiment 79] A process of embodiment 77, wherein R³ is aryl.
[Embodiment 80] A process of embodiment 79, wherein R³ is optionally substituted phenyl.
[Embodiment 81] A process of embodiment 71, wherein R^{4a} and R^{4b} are each independently C₁₋₃alkyl.
[Embodiment 82] A process of embodiment 81, wherein R^{4a} and R^{4b} are methyl.
[Embodiment 83] A process of embodiment 75, wherein each R⁶ is independently H, alkyl, aralkyl, or an hydroxyl protecting group.
[Embodiment 84] A process of embodiment 83, wherein each R⁶ is independently H, aralkyl, or an hydroxyl protecting group.
[Embodiment 85] A process of embodiment 84, wherein each R⁶ is independently H or an hydroxyl protecting group.
[Embodiment 86] A process of embodiment 85, wherein R⁶ is H.
[Embodiment 87] A process of embodiment 71, wherein at least one of R⁷ and R⁸ is H.
[Embodiment 88] A process of embodiment 87, wherein R⁷ and R⁸ are H.
[Embodiment 89] A process of embodiment 71, wherein R⁵ is —C(═O)R^{5a}.
[Embodiment 90] A process of embodiment 89, wherein each R^{5a} is independently alkyl.
[Embodiment 91] A process of embodiment 71, in which the N-alkenylpiperidine compound of Formula IIa is prepared.
[Embodiment 92] A process of embodiment 71, in which a mixture of the N-alkenylpiperidine compounds of Formula IIa and Formula IIb is prepared.
[Embodiment 93] A process of embodiment 71, wherein each R¹ is independently H or C₁₋₆alkyl, R² is —OH, R³ is phenyl, and R^{4a} and R^{4b} are methyl.
[Embodiment 94] A process for preparing an N-alkylpiperidine compound of Formula Va or Formula Vb, or mixture thereof:

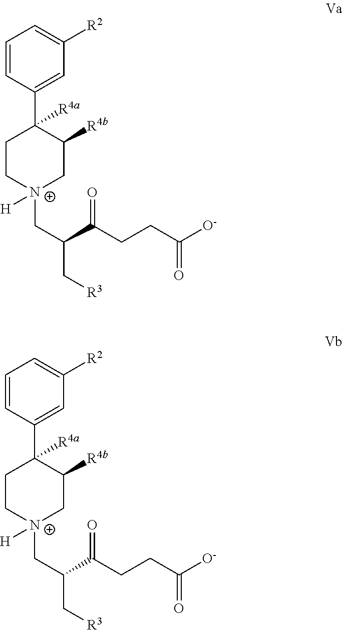

wherein:
each R² is independently Cl, Br, I, —C(═O)OR^{5b}, —CN, —OR⁶, or —CONR⁷R⁸;
each R³ is independently H, alkyl, cycloalkyl, or aryl;
each R^{4a} and R^{4b} is independently C₁₋₆alkyl;
each R^{5b} is independently H or alkyl;
each R⁶ is independently H, alkyl, cycloalkyl, alkylcycloalkyl, aralkyl, or an hydroxyl protecting group;
each R⁷ is independently H, alkyl, cycloalkyl, alkylcycloalkyl, or aralkyl; and
each R⁸ is independently H, alkyl, aralkyl, or aryl;
or a salt thereof;
   comprising providing an N-alkenylpiperidine compound of Formula IIa, Formula IIb, Formula IIc, or Formula IId, or mixture thereof:

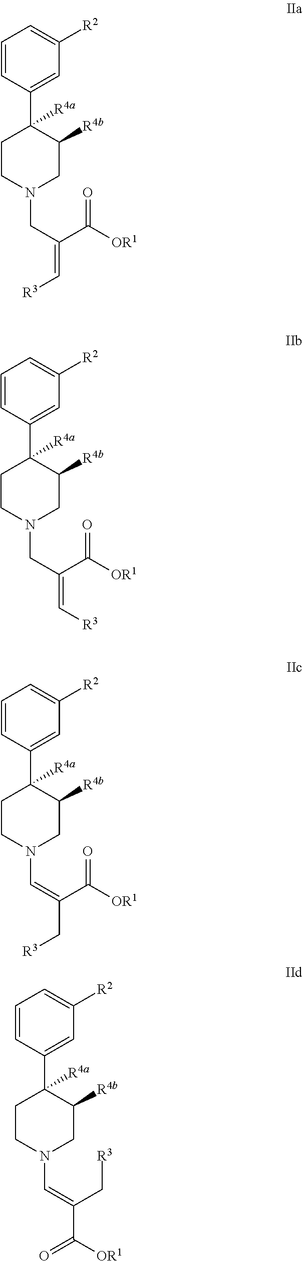

wherein:
R¹ is independently H, alkyl, or aralkyl;
   contacting the N-alkenylpiperidine compound of Formula IIa, Formula IIb, Formula IIc or Formula IId, or mixture thereof, with hydrogen in the presence of a hydrogenation catalyst and a chiral phosphorus-containing ligand for a time and under conditions effective to provide a compound of Formula Ia, a compound of Formula Ib, or mixture thereof:

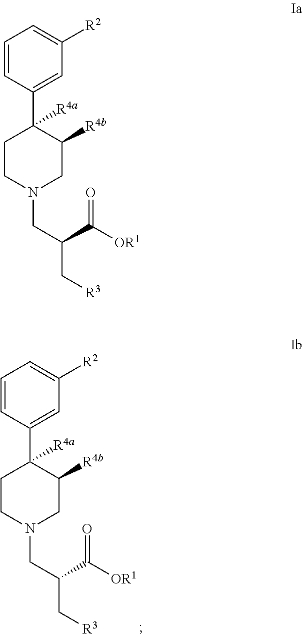

and contacting the compound of Formula Ia, Formula Ib, or mixture thereof, with NH₂CH₂CO₂H for a time and under conditions effective to provide the compound of Formula Va, Formula Vb, or mixture thereof.
[Embodiment 95] A process of embodiment 94, wherein the N-alkenylpiperidine compound of Formula IIa or Formula IIb or mixture thereof is prepared by a process comprising contacting a piperidine compound of Formula III:

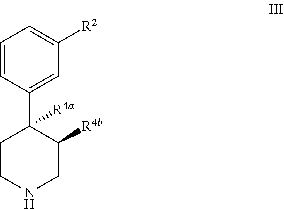

with an alkene compound of Formula IVa or Formula IVb or mixture thereof:

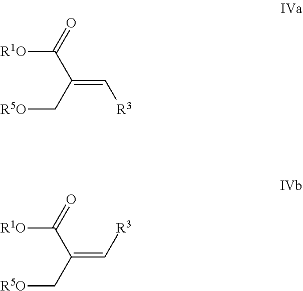

wherein:
each R⁵ is independently alkyl, aralkyl, or —C(═O)R^{5a}; and
each R^{5a} is independently H, alkyl, or aralkyl;
   for a time and under conditions effective to provide the N-alkenylpiperidine compound of Formula IIa or Formula IIb, or mixture thereof.
[Embodiment 96] A process of embodiment 94, wherein the N-alkylpiperidine compounds of Formula Ia and Formula Ib are prepared in a molar ratio of greater than about 1 to about 1.
[Embodiment 97] A process of embodiment 96, wherein the N-alkylpiperidine compounds of Formula Ia and Formula Ib are prepared in a molar ratio of at least about 2 to about 1.
[Embodiment 98] A process of embodiment 97, wherein the N-alkylpiperidine compounds of Formula Ia and Formula Ib are prepared in a molar ratio of at least about 4 to about 1.
[Embodiment 99] A process of embodiment 98, wherein the N-alkylpiperidine compounds of Formula Ia and Formula Ib are prepared in a molar ratio of at least about 9 to about 1.
[Embodiment 100] A process of embodiment 99, wherein the N-alkylpiperidine compounds of Formula Ia and Formula Ib are prepared in a molar ratio of at least about 19 to about 1.
[Embodiment 101] A process of embodiment 94, wherein each R¹ is independently H or C₁₋₆alkyl.
[Embodiment 102] A process of embodiment 101, wherein each R¹ is independently H or CH₃.
[Embodiment 103] A process of embodiment 102, wherein R¹ is H.
[Embodiment 104] A process of embodiment 94, wherein R² is —OR⁶.
[Embodiment 105] A process of embodiment 94, wherein each R3 is independently alkyl, cycloalkyl, or aryl.
[Embodiment 106] A process of embodiment 105, wherein each R³ is independently cycloalkyl or aryl.
[Embodiment 107] A process of embodiment 106, wherein R³ is optionally substituted cyclohexyl.
[Embodiment 108] A process of embodiment 106, wherein R³ is aryl.
[Embodiment 109] A process of embodiment 108, wherein R³ is optionally substituted phenyl.
[Embodiment 110] A process of embodiment 94, wherein R^{4a} and R^{4b} are each independently C₁₋₃alkyl.
[Embodiment 111] A process of embodiment 110, wherein R^{4a} and R^{4b} are methyl.
[Embodiment 112] A process of embodiment 104, wherein each R⁶ is independently H, alkyl, aralkyl, or an hydroxyl protecting group.
[Embodiment 113] A process of embodiment 112, wherein each R⁶ is independently H, aralkyl, or an hydroxyl protecting group.
[Embodiment 114] A process of embodiment 113, wherein each R⁶ is independently H or an hydroxyl protecting group.
[Embodiment 115] A process of embodiment 114, wherein R⁶ is H.
[Embodiment 116] A process of embodiment 94, wherein at least one of R⁷ and R⁸ is H.
[Embodiment 117] A process of embodiment 116, wherein R⁷ and R⁸ are H.
[Embodiment 118] A process of embodiment 97, wherein each R¹ is independently H or C₁₋₆alkyl, R² is —OH, R³ is phenyl, and R^{4a} and R^{4b} are methyl.
[Embodiment 119] A process of embodiment 95, wherein R⁵ is —C(═O)R^{5a}.
[Embodiment 120] A process of embodiment 119, wherein each R^{5a} is independently alkyl.
[Embodiment 121] A process of embodiment 94, wherein the N-alkenylpiperidine compound is an N-alkenylpiperidine compound of Formula IIa or Formula IIb, or mixture thereof.
[Embodiment 122] A process of embodiment 121, wherein the N-alkenylpiperidine compound is an N-alkenylpiperidine compound of Formula IIa.
[Embodiment 123] A process of embodiment 121, wherein the N-alkenylpiperidine compound comprises a mixture of N-alkenylpiperidine compounds of Formula IIa and Formula IIb.
[Embodiment 124] A process of embodiment 1, wherein the N-alkenylpiperidine compound of Formula IIa, Formula IIb, Formula IIc or Formula IId, or mixture thereof, is contacted with hydrogen at a temperature of from about 10° C. to about 100° C.
[Embodiment 125] A process of embodiment 124, wherein the temperature is from about 20° C. to about 85° C.
[Embodiment 126] A process of embodiment 125, wherein the temperature is from about 20° C. to about 65° C.
[Embodiment 127] A process of embodiment 94, wherein the N-alkenylpiperidine compound of Formula IIa, Formula IIb, Formula IIc or Formula IId, or mixture thereof, is contacted with hydrogen in a reactor into which hydrogen gas is charged at a pressure of from about 1 bar to about 150 bar.
[Embodiment 128] A process of embodiment 127, wherein hydrogen gas is charged into the reactor at a pressure of from about 1 bar to about 80 bar.
[Embodiment 129] A process of embodiment 128, wherein hydrogen gas is charged into the reactor at a pressure of from about 3 bar to about 50 bar.
[Embodiment 130] A process of embodiment 129, wherein hydrogen gas is charged into the reactor at a pressure of from about 3 bar to about 30 bar.
[Embodiment 131] A process of embodiment 94, wherein the molar ratio of the N-alkenylpiperidine compound to the hydrogenation catalyst is from about 10 to about 50,000.
[Embodiment 132] A process of embodiment 131, wherein the molar ratio of the N-alkenylpiperidine compound to the hydrogenation catalyst is from about 100 to about 10,000.
[Embodiment 133] A process of embodiment 132, wherein the molar ratio of the N-alkenylpiperidine compound to the hydrogenation catalyst is from about 100 to about 2,000.
[Embodiment 134] A process of embodiment 94, wherein the N-alkenylpiperidine compound of Formula IIa, Formula IIb, Formula IIc or Formula IId, or mixture thereof, is contacted with hydrogen for about 1 to about 100 hours.
[Embodiment 135] A process of embodiment 134, wherein the contacting time is from about 1 to about 24 hours.
[Embodiment 136] A process of embodiment 135, wherein the contacting time is from about 1 to about 10 hours.
[Embodiment 137] A process of embodiment 94, wherein the hydrogenation catalyst is heterogeneous.
[Embodiment 138] A process of embodiment 137, wherein the heterogeneous hydrogenation catalyst comprises palladium.
[Embodiment 139] A process of embodiment 94, wherein the hydrogenation catalyst is homogeneous.
[Embodiment 140] A process of embodiment 139, wherein the homogeneous hydrogenation catalyst comprises a Group VIII transition metal.
[Embodiment 141] A process of embodiment 140, wherein the Group VIII transition metal catalyst comprises rhodium, ruthenium, or iridium.
[Embodiment 142] A process of embodiment 141, wherein the Group VIII transition metal catalyst comprises rhodium.
[Embodiment 143] A process of embodiment 94, wherein the chiral phosphorus-containing ligand is a chiral tertiary diphosphine.
[Embodiment 144] A process of embodiment 143, wherein the chiral tertiary diphosphine is selected from the group consisting of:

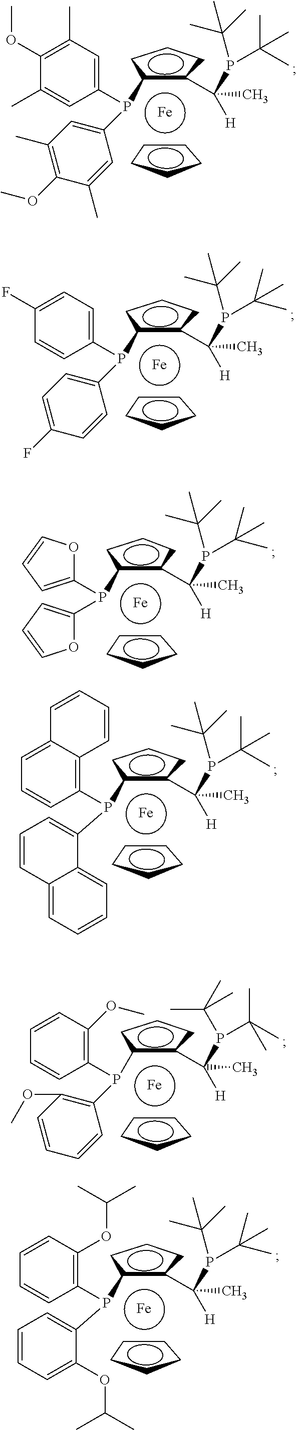

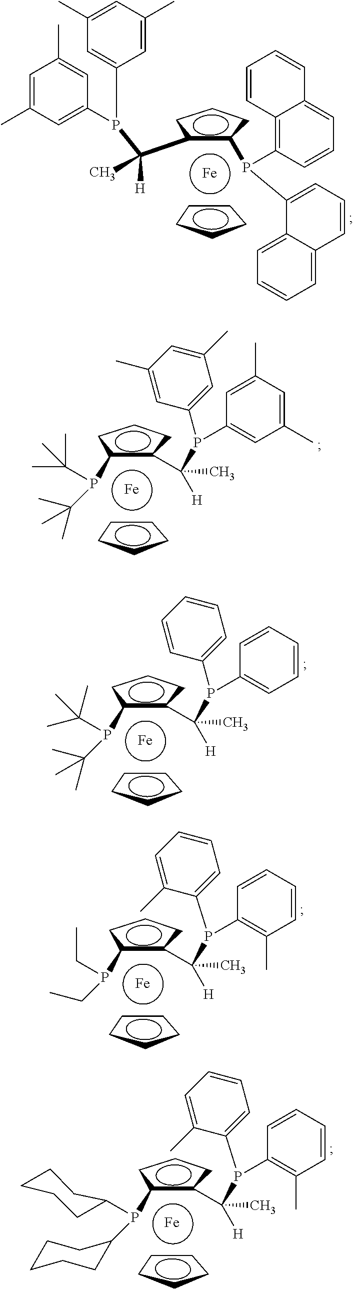

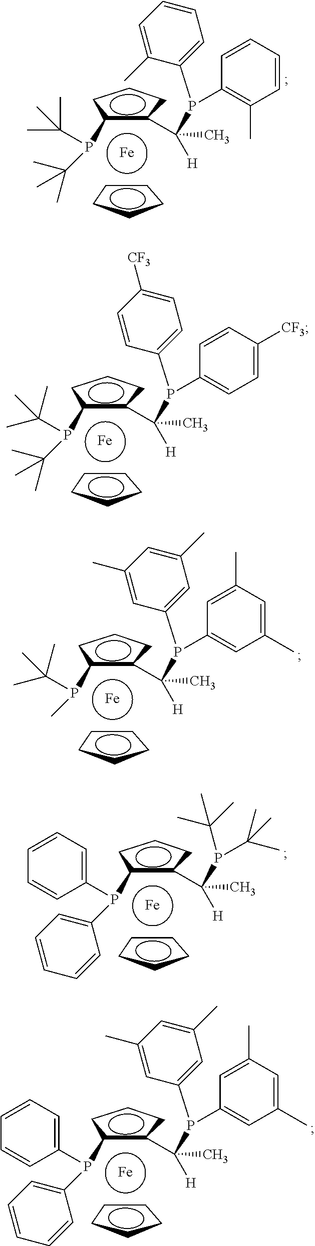

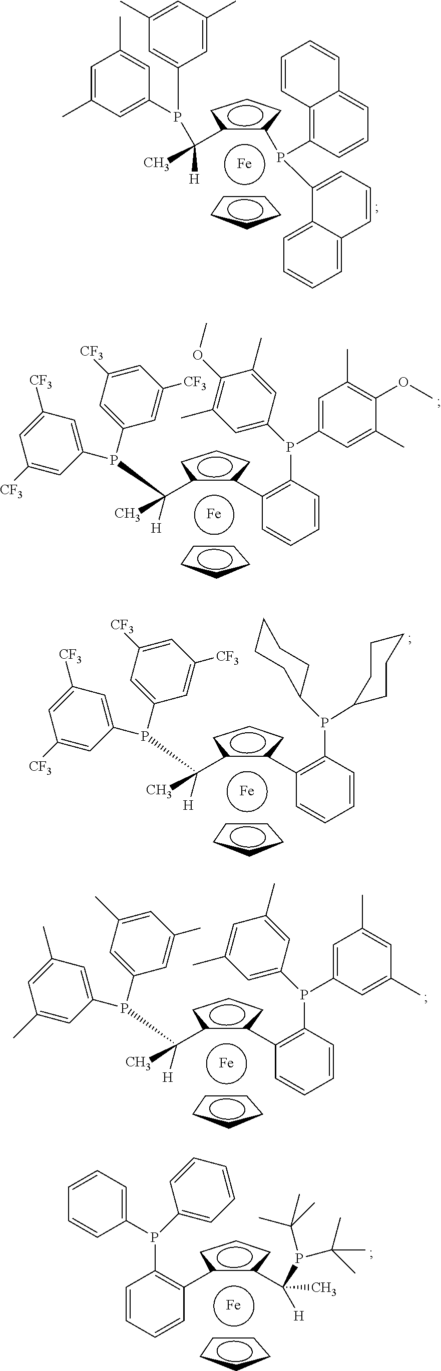

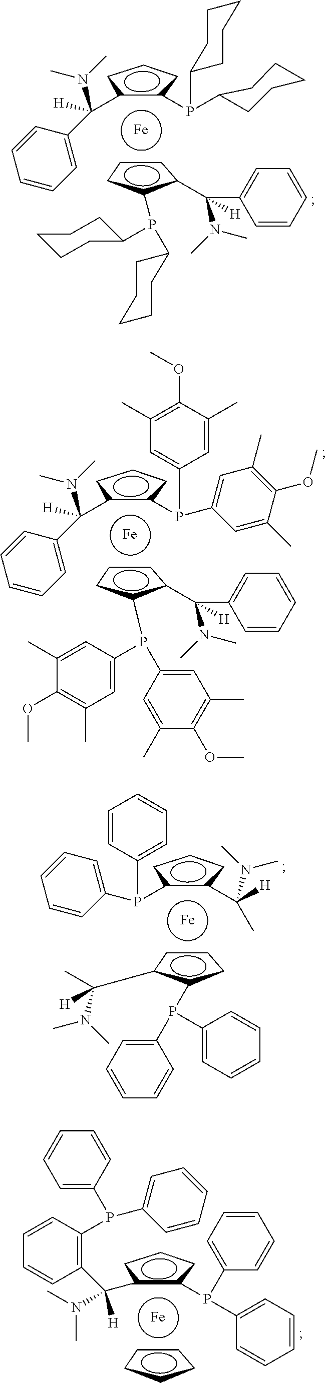

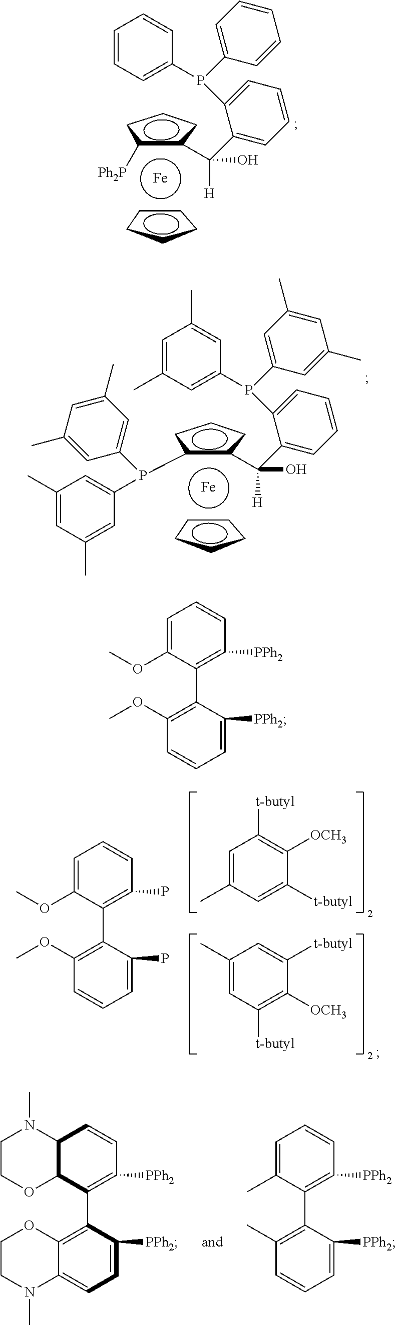

or a mixture thereof.
[Embodiment 145] A process of embodiment 144, wherein the chiral tertiary diphosphine is selected from the group consisting of:

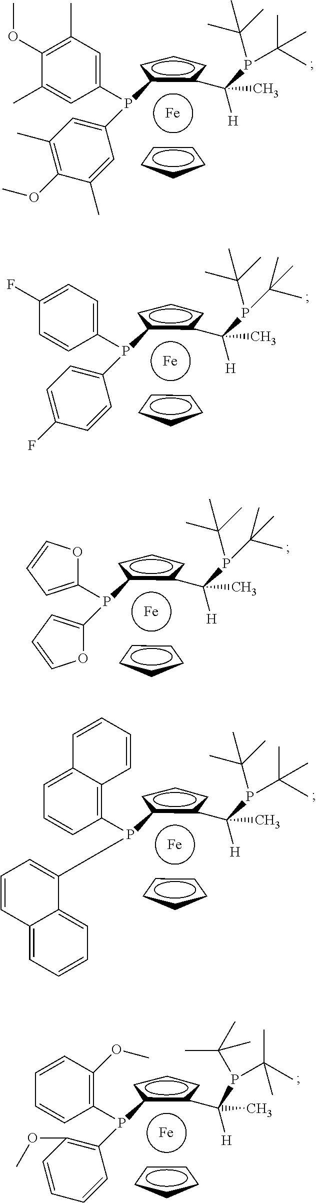

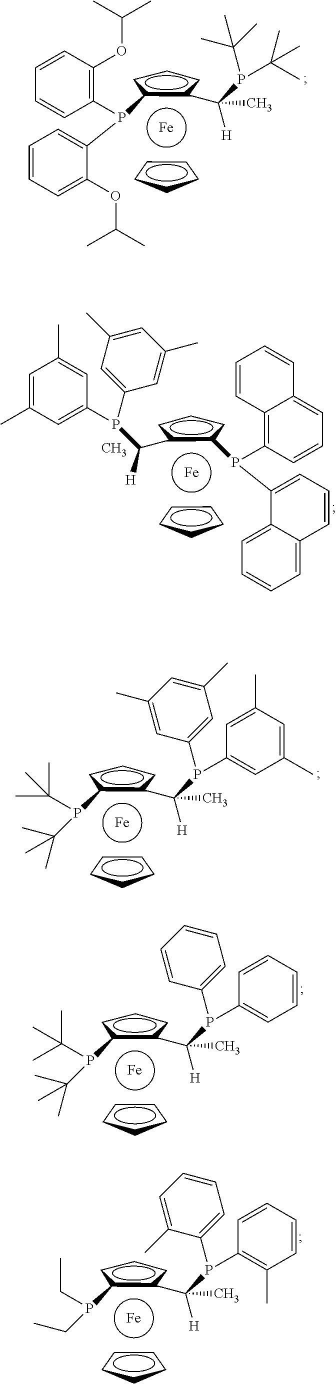

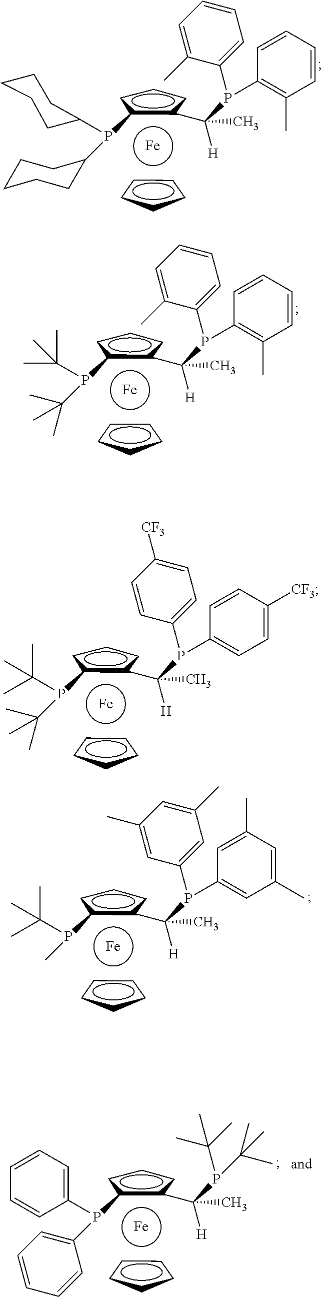

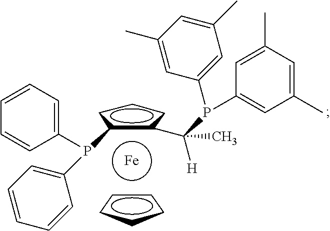

or a mixture thereof.
[Embodiment 146] A process of embodiment 144, wherein the chiral tertiary diphosphine is selected from the group consisting of:

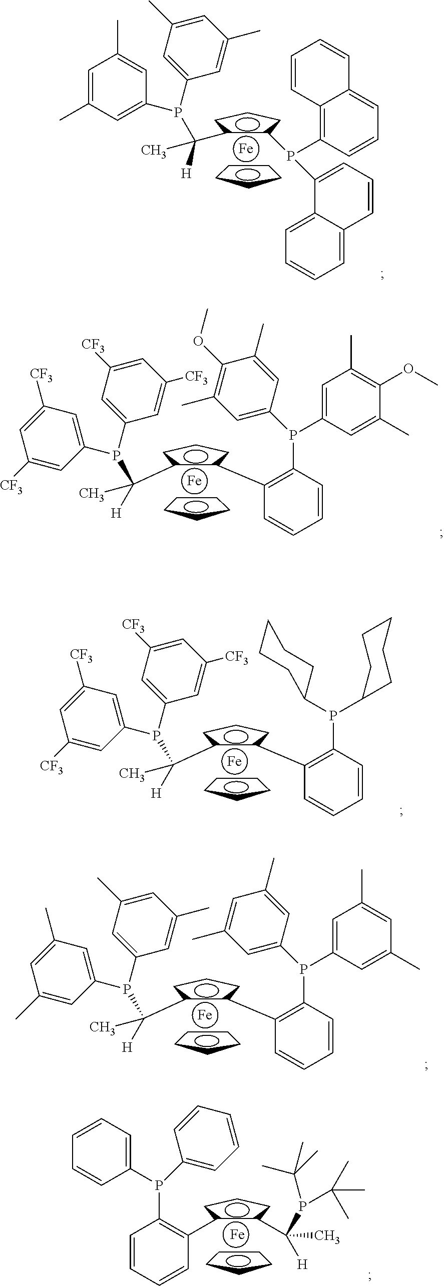

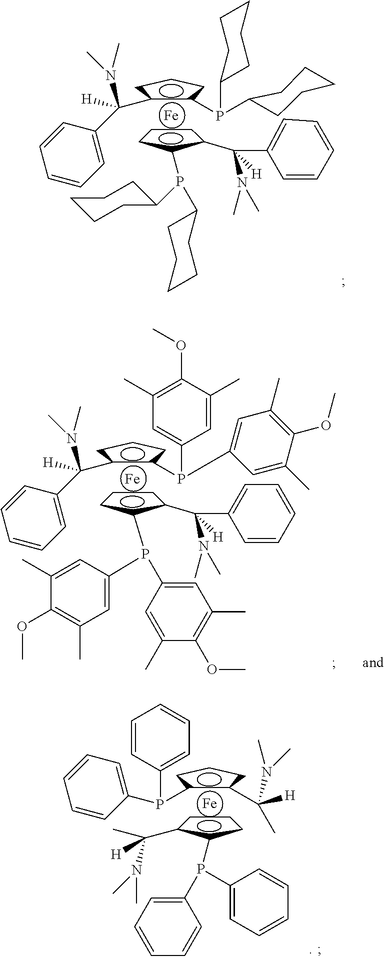

or a mixture thereof.
[Embodiment 147] A process of embodiment 144, wherein the chiral tertiary diphosphine is selected from the group consisting of:

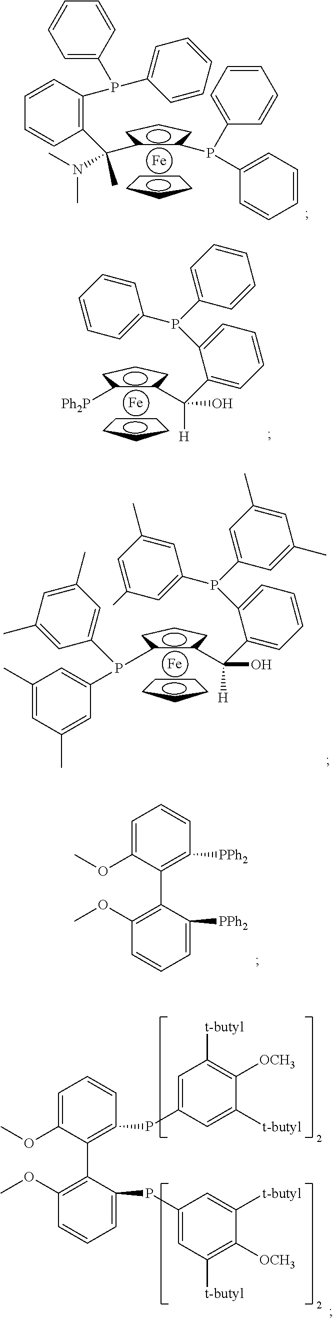

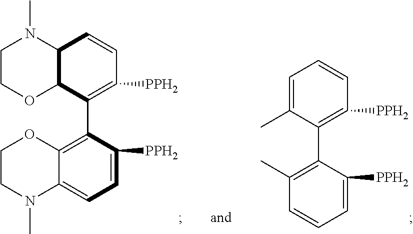

or a mixture thereof.
[Embodiment 148] A process of embodiment 94, wherein the N-alkenylpiperidine compound of Formula IIa, Formula IIb, Formula IIc or Formula IId, or mixture thereof, is contacted with hydrogen in a solvent.
[Embodiment 149] A process of embodiment 148, wherein the solvent comprises an alcoholic solvent, an ether, an aromatic hydrocarbon, a chlorinated hydrocarbon, an ester, a lactone, or mixture thereof.
[Embodiment 150] A process of embodiment 149, wherein the solvent comprises an ether.
[Embodiment 151] A process of embodiment 150, wherein the solvent comprises tetrahydrofuran or dioxane or a mixture thereof.
[Embodiment 152] A process of embodiment 149, wherein the solvent comprises an alcoholic solvent.
[Embodiment 153] A process of embodiment 152, wherein the solvent further comprises water.
[Embodiment 154] A process of embodiment 152, wherein the alcoholic solvent comprises a C₁₋₃alkanol.
[Embodiment 155] A process of embodiment 154, wherein the alcoholic solvent comprises methanol.
[Embodiment 156] A process of embodiment 94, wherein the N-alkenylpiperidine compound of Formula IIa, Formula IIb, Formula IIc or Formula IId, or mixture thereof, is contacted with hydrogen in the presence of an additive.
[Embodiment 157] A process of embodiment 156, wherein the additive is a proton acid or an amine.
[Embodiment 158] A process of embodiment 157, wherein the additive is an amine.
[Embodiment 159] A process of embodiment 158, wherein the amine is N,N,N,N-tetramethylguanidine.
[Embodiment 160] A process of embodiment 157, wherein the additive is a proton acid.
[Embodiment 161] A process of embodiment 160, wherein the proton acid is selected from the group consisting of an alkylsulfonic acid, an arylsulfonic acid, sulfuric acid, hydrochloric acid, and a carboxylic acid.
[Embodiment 162] A process of embodiment 161, wherein the proton acid is selected from the group consisting of an alkylsulfonic acid and an arylsulfonic acid.
[Embodiment 163] A process of embodiment 162, wherein the proton acid is methanesulfonic acid.
[Embodiment 164] A compound of Formula IIa:

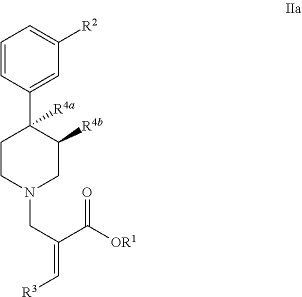

wherein:
R¹ is H, alkyl, or aralkyl;
R² is Cl, Br, I, —C(═O)OR^{5b}, —CN, —OR⁶, or —CONR⁷R⁸;
R³ is H, alkyl, cycloalkyl, or aryl;
R^{4a} and R^{4b} are each independently C₁₋₆alkyl;
R^{5b} is independently H or alkyl;
R⁶ is H, alkyl, cycloalkyl, alkylcycloalkyl, aralkyl, or an hydroxyl protecting group;
R⁷ is H, alkyl, cycloalkyl, alkylcycloalkyl, or aralkyl; and
R⁸ is H, alkyl, aralkyl, or aryl;
or a salt thereof.
[Embodiment 165] A compound of embodiment 164, wherein R¹ is H or C₁₋₆alkyl.
[Embodiment 166] A compound of embodiment 165, wherein R¹ is H or CH₃.
[Embodiment 167] A compound of embodiment 166, wherein R¹ is H.
[Embodiment 168] A compound of embodiment 164, wherein R² is —OR⁶.
[Embodiment 169] A compound of embodiment 164, wherein R³ is alkyl, cycloalkyl, or aryl.
[Embodiment 170] A compound of embodiment 169, wherein R³ is cycloalkyl or aryl.
[Embodiment 171] A compound of embodiment 170, wherein R³ is optionally substituted cyclohexyl.
[Embodiment 172] A compound of embodiment 170, wherein R³ is aryl.
[Embodiment 173] A compound of embodiment 172, wherein R³ is optionally substituted phenyl.
[Embodiment 174] A compound of embodiment 164, wherein R^{4a} and R^{4b} are each independently C₁₋₃alkyl.
[Embodiment 175] A compound of embodiment 174, wherein R^{4a} and R^{4b} are methyl.
[Embodiment 176] A compound of embodiment 168, wherein R⁶ is H, alkyl, aralkyl, or an hydroxyl protecting group.
[Embodiment 177] A compound of embodiment 176, wherein R⁶ is H, aralkyl, or an hydroxyl protecting group.
[Embodiment 178] A compound of embodiment 177, wherein R⁶ is H or an hydroxyl protecting group.
[Embodiment 179] A compound of embodiment 178, wherein R⁶ is H.
[Embodiment 180] A compound of embodiment 164, wherein at least one of R⁷ and R⁸ is H.
[Embodiment 181] A compound of embodiment 180, wherein R⁷ and R⁸ are H.
[Embodiment 182] A compound of embodiment 164, wherein each R¹ is independently H or C₁₋₆alkyl, R² is —OH, R³ is phenyl, and R^{4a} and R^{4b} are methyl.
[Embodiment 183] A compound of Formula IIb:

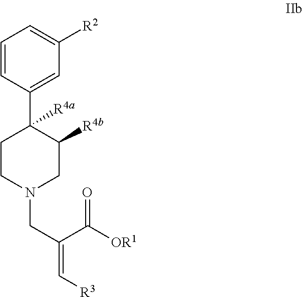

wherein:
R¹ is H, alkyl, or aralkyl;
R² is Cl, Br, I, —C(═O)OR^{5b}, —CN, —OR⁶, or —CONR⁷R⁸;
R³ is H, alkyl, cycloalkyl, or aryl;
R^{4a} and R^{4b} are each independently C₁₋₆alkyl;
R^{5b} is independently H or alkyl;
R⁶ is H, alkyl, cycloalkyl, alkylcycloalkyl, aralkyl, or an hydroxyl protecting group;
R⁷ is H, alkyl, cycloalkyl, alkylcycloalkyl, or aralkyl; and
R⁸ is H, alkyl, aralkyl, or aryl;
or a salt thereof.
[Embodiment 184] A compound of embodiment 183, wherein R¹ is H or C₁₋₆alkyl.
[Embodiment 185] A compound of embodiment 184, wherein R¹ is H or CH₃.
[Embodiment 186] A compound of embodiment 185, wherein R¹ is H.
[Embodiment 187] A compound of embodiment 183, wherein R² is —OR⁶.
[Embodiment 188] A compound of embodiment 183, wherein R³ is alkyl, cycloalkyl, or aryl.
[Embodiment 189] A compound of embodiment 188, wherein R³ is cycloalkyl or aryl.
[Embodiment 190] A compound of embodiment 189, wherein R³ is optionally substituted cyclohexyl.
[Embodiment 191] A compound of embodiment 189, wherein R³ is aryl.
[Embodiment 192] A compound of embodiment 191, wherein R³ is optionally substituted phenyl.
[Embodiment 193] A compound of embodiment 183, wherein R^{4a} and R^{4b} are each independently C₁₋₃alkyl.
[Embodiment 194] A compound of embodiment 193, wherein R^{4a} and R^{4b} are methyl.
[Embodiment 195] A compound of embodiment 187, wherein R⁶ is H, alkyl, aralkyl, or an hydroxyl protecting group.
[Embodiment 196] A compound of embodiment 195, wherein R⁶ is H, aralkyl, or an hydroxyl protecting group.
[Embodiment 197] A compound of embodiment 196, wherein R⁶ is H or an hydroxyl protecting group.
[Embodiment 198] A compound of embodiment 197, wherein R⁶ is H.
[Embodiment 199] A compound of embodiment 183, wherein at least one of R⁷ and R⁸ is H.
[Embodiment 200] A compound of embodiment 199, wherein R⁷ and R⁸ are H.
[Embodiment 201] A compound of embodiment 183, wherein each R¹ is independently H or C₁₋₆alkyl, R² is —OH, R³ is phenyl, and R^{4a} and R^{4b} are methyl.
[Embodiment 202] A compound of Formula IIc:

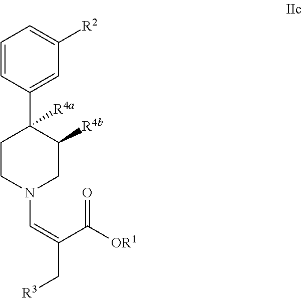

wherein:
R¹ is H, alkyl, or aralkyl;
R² is Cl, Br, I, —C(═O)OR^{5b}, —CN, —OR⁶, or —CONR⁷R⁸;
R³ is H, alkyl, cycloalkyl, or aryl;
R^{4a} and R^{4b} are each independently C₁₋₆alkyl;
R^{5b} is independently H or alkyl;
R⁶ is H, alkyl, cycloalkyl, alkylcycloalkyl, aralkyl, or an hydroxyl protecting group;
R⁷ is H, alkyl, cycloalkyl, alkylcycloalkyl, or aralkyl; and
R⁸ is H, alkyl, aralkyl, or aryl;
or a salt thereof.
[Embodiment 203] A compound of embodiment 202, wherein R¹ is H or C₁₋₆alkyl.
[Embodiment 204] A compound of embodiment 203, wherein R³ is H or CH₃.
[Embodiment 205] A compound of embodiment 204, wherein R¹ is H.
[Embodiment 206] A compound of embodiment 202, wherein R² is —OR⁶.
[Embodiment 207] A compound of embodiment 202, wherein R³ is alkyl, cycloalkyl, or aryl.
[Embodiment 208] A compound of embodiment 207, wherein R³ is cycloalkyl or aryl.
[Embodiment 209] A compound of embodiment 208, wherein R³ is optionally substituted cyclohexyl.
[Embodiment 210] A compound of embodiment 208, wherein R³ is aryl.
[Embodiment 211] A compound of embodiment 210, wherein R³ is optionally substituted phenyl.
[Embodiment 212] A compound of embodiment 202, wherein R^{4a} and R^{4b} are each independently C₁₋₃alkyl.
[Embodiment 213] A compound of embodiment 212, wherein R^{4a} and R^{4b} are methyl.
[Embodiment 214] A compound of embodiment 206, wherein R⁶ is H, alkyl, aralkyl, or an hydroxyl protecting group.
[Embodiment 215] A compound of embodiment 214, wherein R⁶ is H, aralkyl, or an hydroxyl protecting group.
[Embodiment 216] A compound of embodiment 215, wherein R⁶ is H or an hydroxyl protecting group.
[Embodiment 217] A compound of embodiment 216, wherein R⁶ is H.
[Embodiment 218] A compound of embodiment 202, wherein at least one of R⁷ and R⁸ is H.
[Embodiment 219] A compound of embodiment 218, wherein R⁷ and R⁸ are H.
[Embodiment 220] A compound of embodiment 202, wherein each R¹ is independently H or C₁₋₆alkyl, R² is —OH, R³ is phenyl, and R^{4a} and R^{4b} are methyl.
[Embodiment 221] A compound of Formula IId:

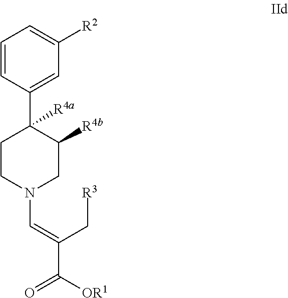

wherein:
R¹ is H, alkyl, or aralkyl;
R² is Cl, Br, I, —C(═O)OR^{5b}, —CN, —OR⁶, or —CONR⁷R⁸;
R³ is H, alkyl, cycloalkyl, or aryl;
R^{4a} and R^{4b} are each independently C₁₋₆alkyl;
R^{5b} is independently H or alkyl;
R⁶ is H, alkyl, cycloalkyl, alkylcycloalkyl, aralkyl, or an hydroxyl protecting group;
R⁷ is H, alkyl, cycloalkyl, alkylcycloalkyl, or aralkyl; and
R⁸ is H, alkyl, aralkyl, or aryl;
or a salt thereof.
[Embodiment 222] A compound of embodiment 221, wherein R¹ is H or C₁₋₆alkyl.
[Embodiment 223] A compound of embodiment 222, wherein R¹ is H or CH3.
[Embodiment 224] A compound of embodiment 223, wherein R¹ is H.
[Embodiment 225] A compound of embodiment 221, wherein R² is —OR⁶.
[Embodiment 226] A compound of embodiment 221, wherein R³ is alkyl, cycloalkyl, or aryl.
[Embodiment 227] A compound of embodiment 226, wherein R³ is cycloalkyl or aryl.
[Embodiment 228] A compound of embodiment 227, wherein R³ is optionally substituted cyclohexyl.
[Embodiment 229] A compound of embodiment 227, wherein R³ is aryl.
[Embodiment 230] A compound of embodiment 229, wherein R³ is optionally substituted phenyl.
[Embodiment 231] A compound of embodiment 221, wherein R^{4a} and R^{4b} are each independently C₁₋₃alkyl.
[Embodiment 232] A compound of embodiment 231, wherein R^{4a} and R^{4b} are methyl.
[Embodiment 233] A compound of embodiment 225, wherein R⁶ is H, alkyl, aralkyl, or an hydroxyl protecting group.
[Embodiment 234] A compound of embodiment 233, wherein R⁶ is H, aralkyl, or an hydroxyl protecting group.
[Embodiment 235] A compound of embodiment 234, wherein R⁶ is H or an hydroxyl protecting group.
[Embodiment 236] A compound of embodiment 235, wherein R⁶ is H.
[Embodiment 237] A compound of embodiment 221, wherein at least one of R⁷ and R⁸ is H.
[Embodiment 238] A compound of embodiment 237, wherein R⁷ and R⁸ are H.
[Embodiment 239] A compound of embodiment 221, wherein each R¹ is independently H or C₁₋₆alkyl, R² is —OH, R³ is phenyl, and R^{4a} and R^{4b} are methyl.
[Embodiment 240] A process according to embodiment 50, wherein the chiral tertiary diphosphine or mixture thereof is selected from the group consisting of:

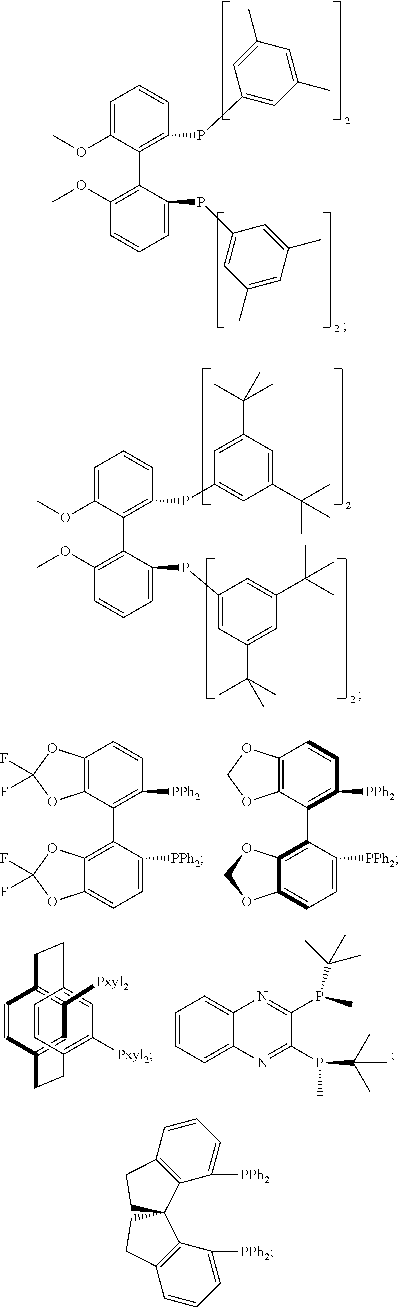

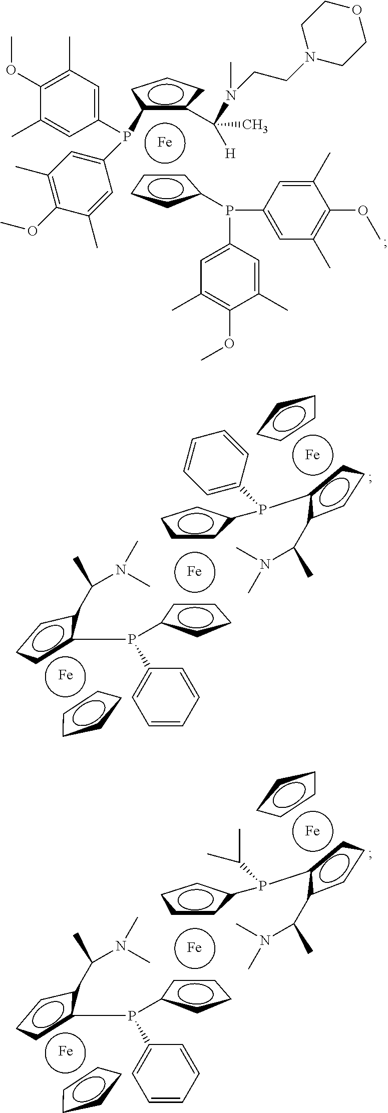

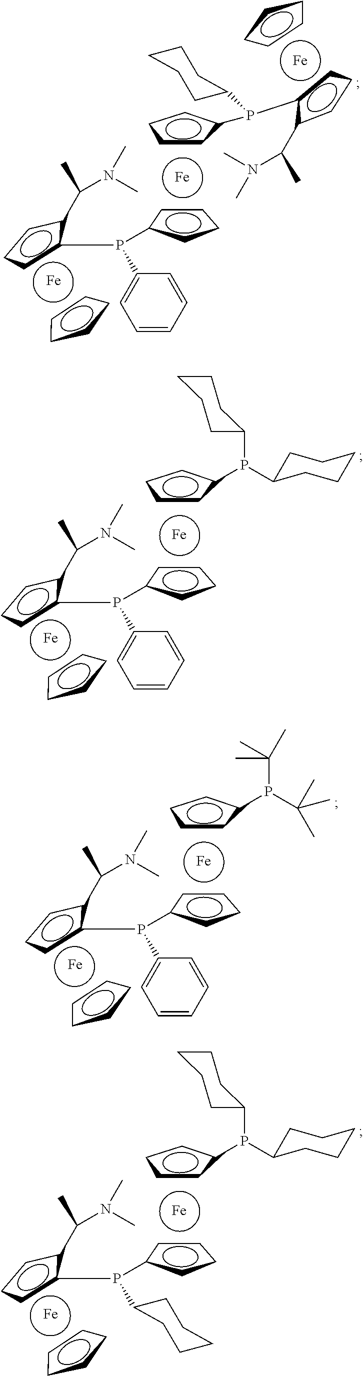

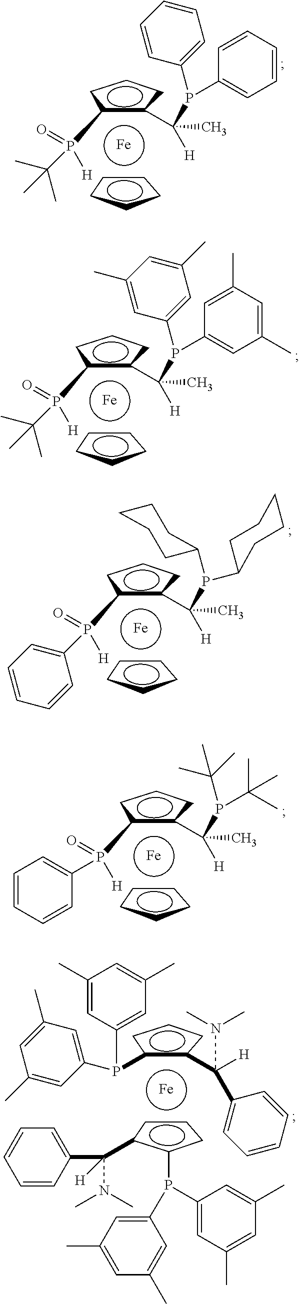

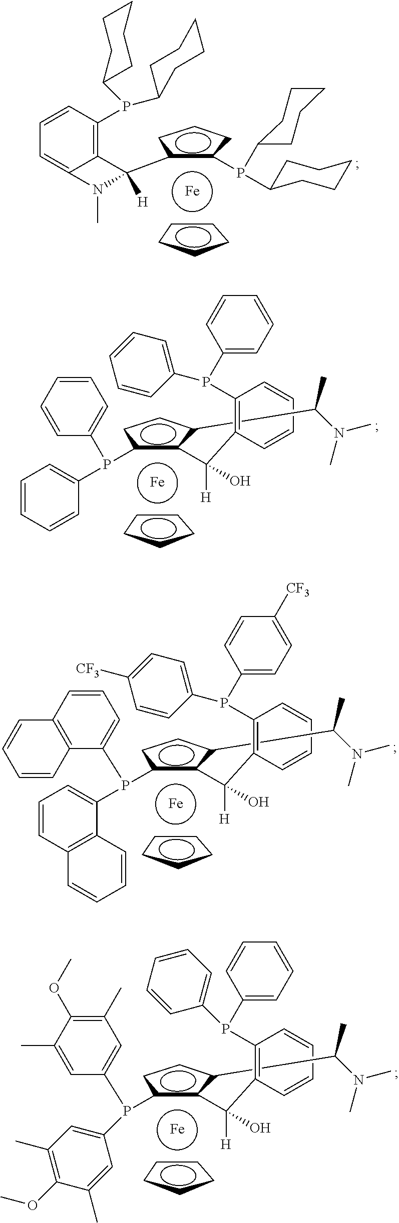

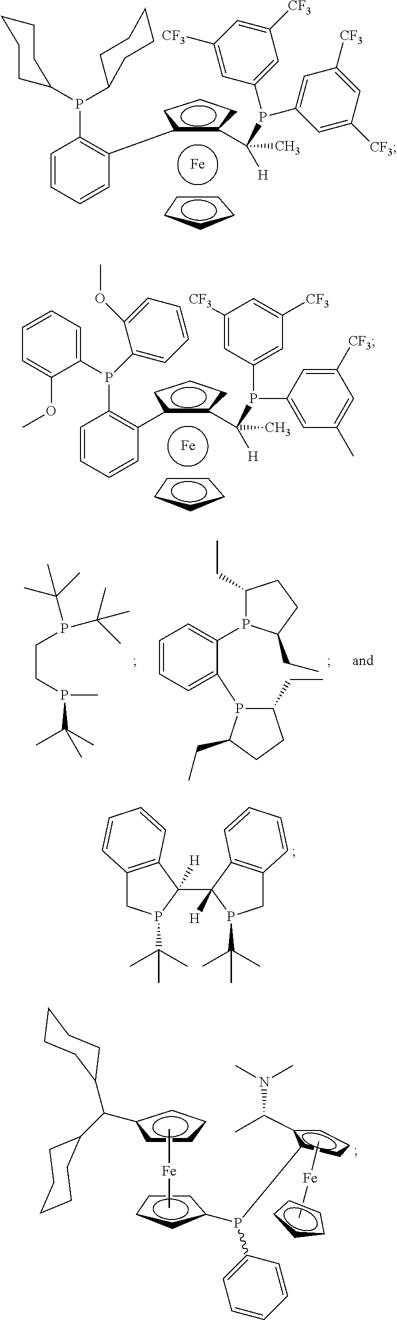

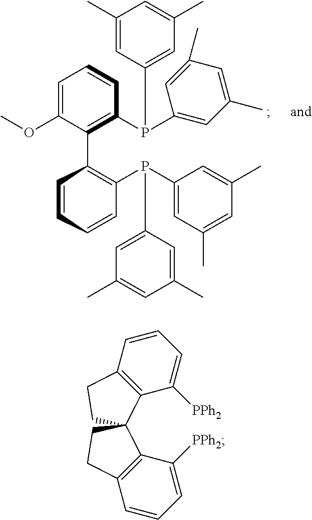

or a mixture thereof.
[Embodiment 241] A process of embodiment 50, wherein the chiral tertiary diphosphine or mixture thereof is selected from the group consisting of:

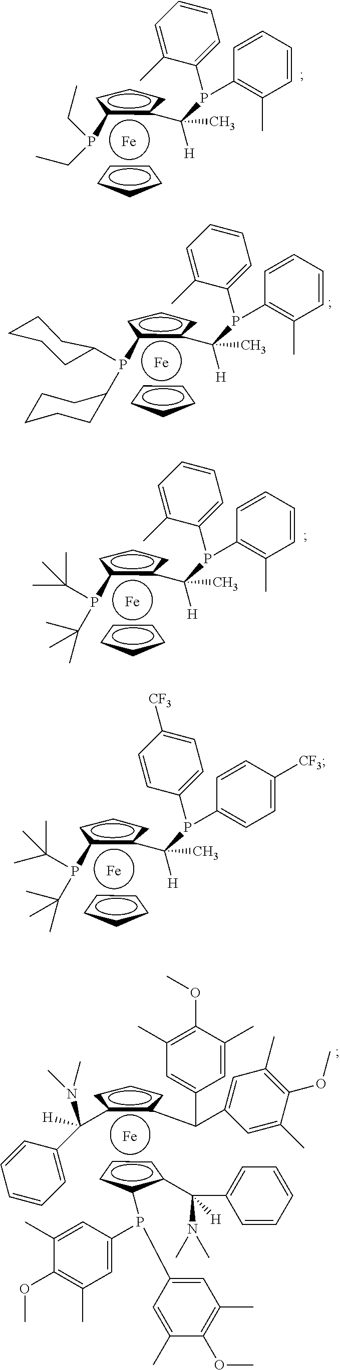

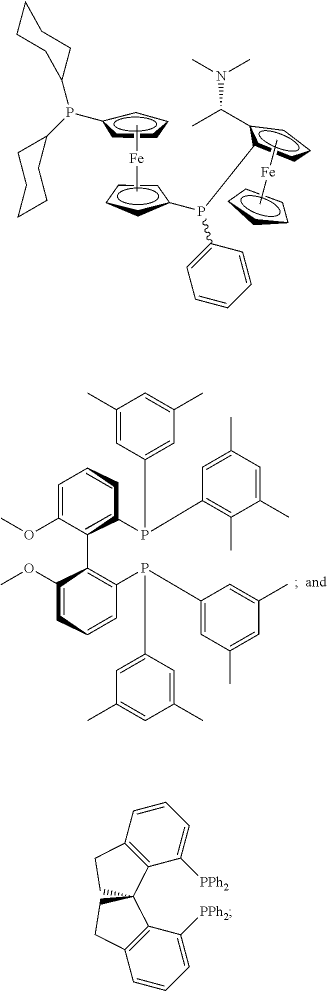

or a mixture thereof.
[Embodiment 242] A process of embodiment 57, wherein the ether comprises 2-methyltetrahydrofuran.
[Embodiment 243] A process of embodiment 61, wherein the alcoholic solvent comprises 2,2,2-trifluoroethanol.
[Embodiment 244] A process of embodiment 68, wherein the carboxylic acid is trifluoroacetic acid.
[Embodiment 245] A process of embodiment 143, wherein the chiral tertiary diphosphine or mixture thereof is selected from the group consisting of:

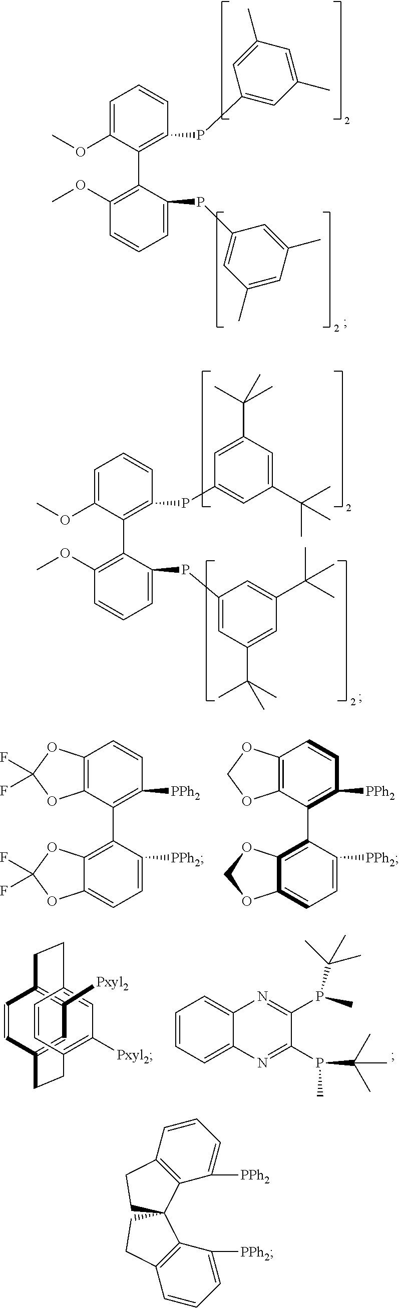

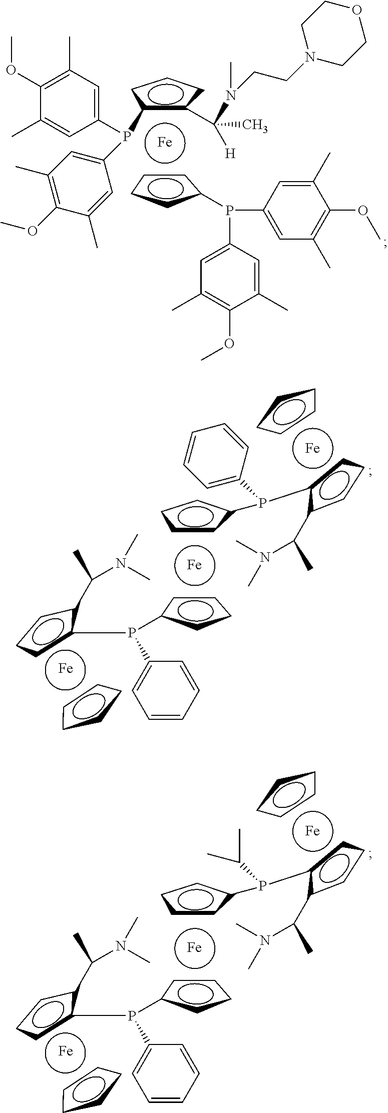

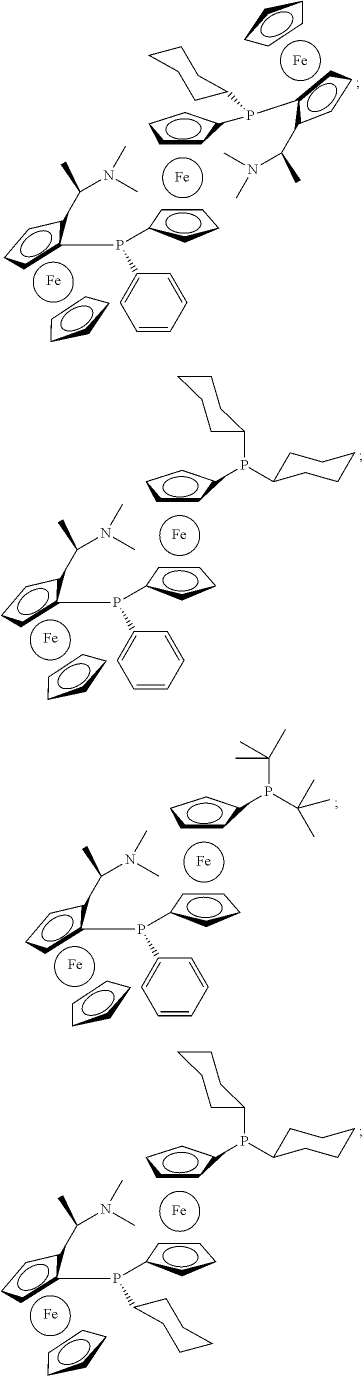

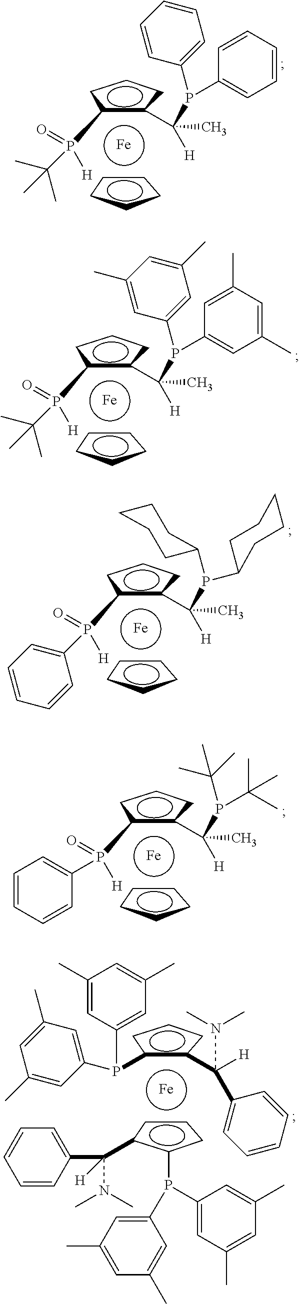

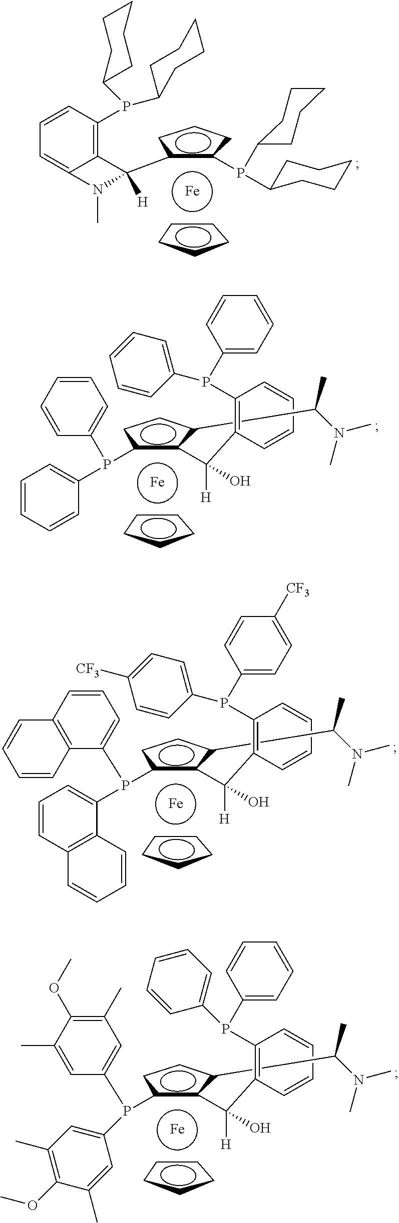

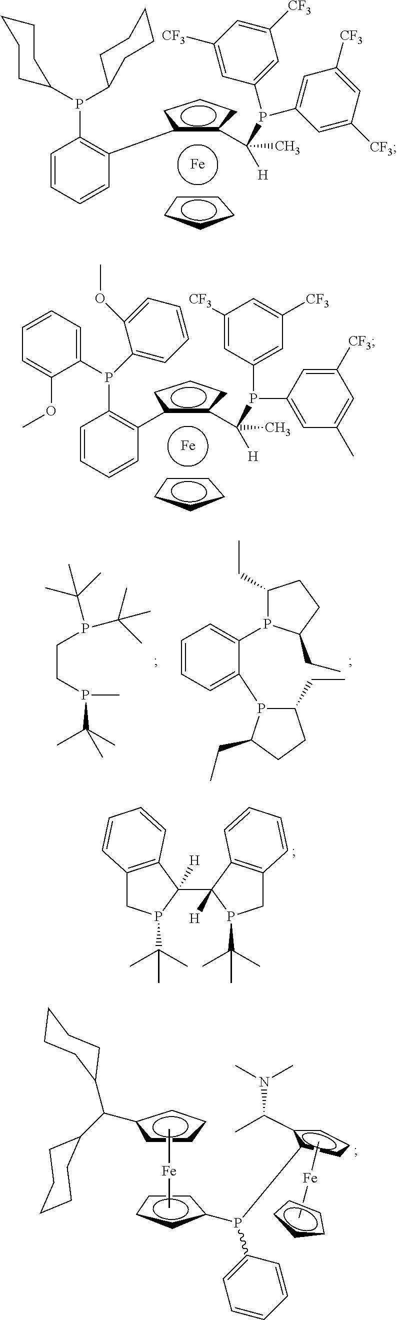

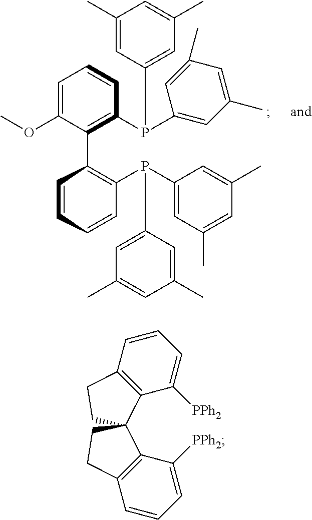

or a mixture thereof.
[Embodiment 246] A process of embodiment 143, wherein the chiral tertiary diphosphine or mixture thereof is selected from the group consisting of:

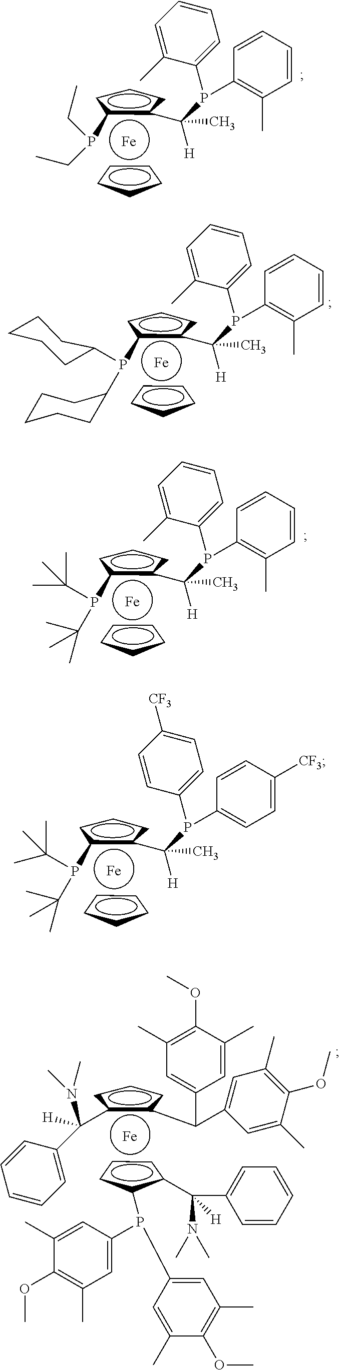

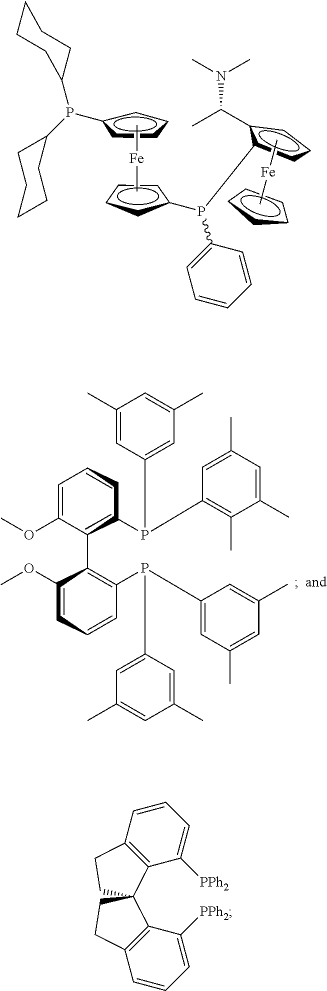

or a mixture thereof.
[Embodiment 247] A process of embodiment 150, wherein the ether comprises 2-methyltetrahydrofuran.
[Embodiment 248] A process of embodiment 154, wherein the alcoholic solvent comprises 2,2,2-trifluoroethanol.
[Embodiment 249] A process of embodiment 161, wherein the carboxylic acid is trifluoroacetic acid.

What is claimed is:

1. A process for preparing an N-alkylpiperidine compound of Formula Ia′, or a pharmaceutically acceptable salt thereof: 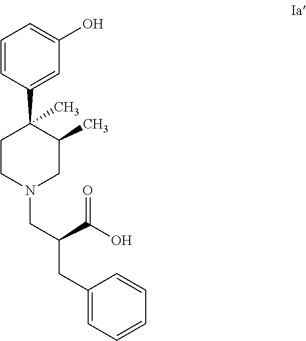 [SMILES: C[C@H]1CN(C[C@H](Cc2ccccc2)C(=O)O)CC[C@@]1(C)c1cccc(O)c1]
comprising contacting an N-alkenylpiperidine compound of Formula IIa′ or Formula IIb′ or mixture thereof: 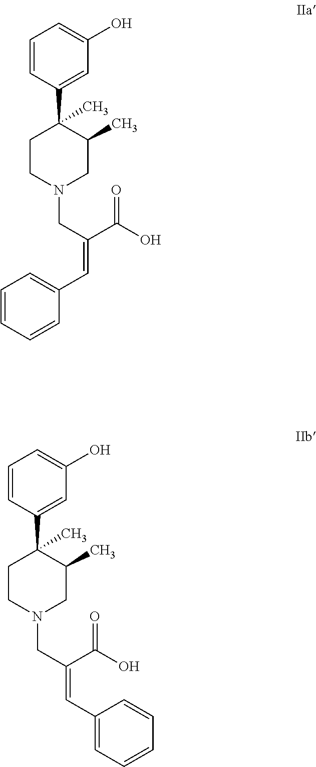 [SMILES: C[C@H]1CN(C/C(=C\c2ccccc2)C(=O)O)CC[C@@]1(C)c1cccc(O)c1 ; C[C@H]1CN(C/C(=C/c2ccccc2)C(=O)O)CC[C@@]1(C)c1cccc(O)c1]
with hydrogen in the presence of a Group VIII transition metal hydrogenation catalyst and a chiral-phosphorous containing ligand for a time and under conditions effective to provide a compound of Formula Ia′.

2. The process according to claim 1, wherein the N-alkylpiperidine is a compound of Formula IIa′.

3. The process according to claim 1, further comprising contacting a compound of Formula Ia′ with an alkylating agent to provide a compound of Formula Ia″: 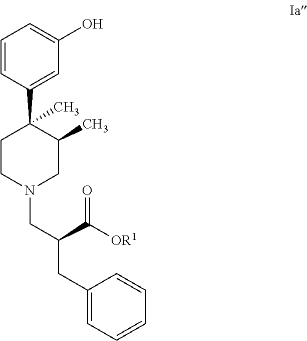
wherein R¹ is alkyl or aralkyl.

4. The process according to claim 3, wherein R¹ is C₁₋₆ alkyl.

5. The process according to claim 4, wherein R¹ is methyl.

6. The process according to claim 3, further comprising contacting the compound of Formula Ia″ with NH₂CH₂CO₂H for a time and under conditions effective to provide (+)-2-[(S)-benzyl-3-[4(R)-(3-hydroxyphenyl)-3(R),4-dimethylpiperidin-1-yl] propionamidolacetic acid (alvimopam), or a pharmaceutically acceptable salt thereof.

7. The process according to claim 1, wherein the Group VIII transition metal catalyst comprises rhodium, ruthenium, or iridium.

8. The process according to claim 7, wherein the Group VIII transition metal catalyst comprises rhodium.

9. The process according to claim 8, wherein the catalyst is [Rhnbd)₂]BF₄.

10. The process according to claim 7, wherein the catalyst is [Ru(cod)(OOCCF₃)₂].

11. The process according to claim 1, wherein the chiral phosphorus-containing ligand is a chiral tertiary diphosphine.

12. The process according to claim 11, wherein the chiral tertiary diphosphine is selected from the group consisting of: 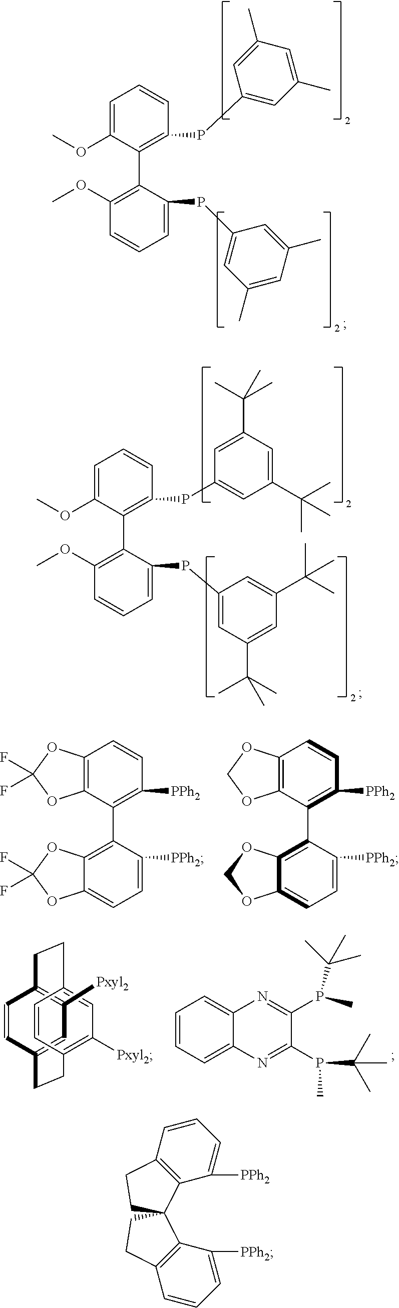 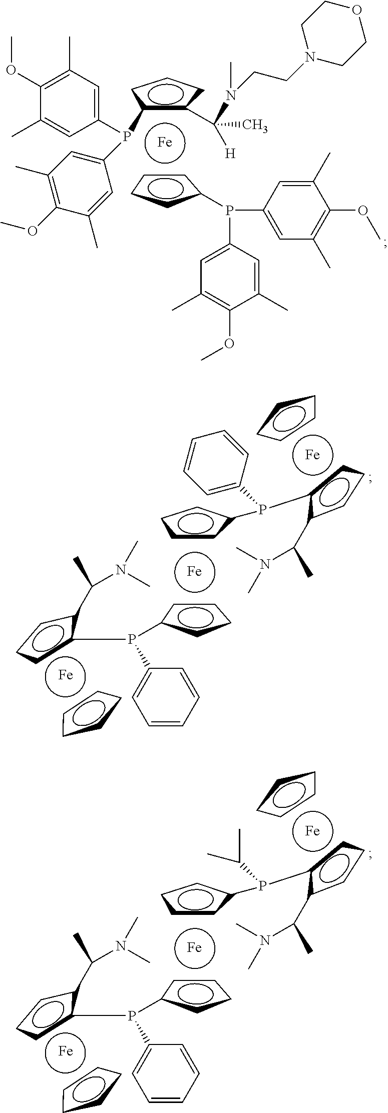 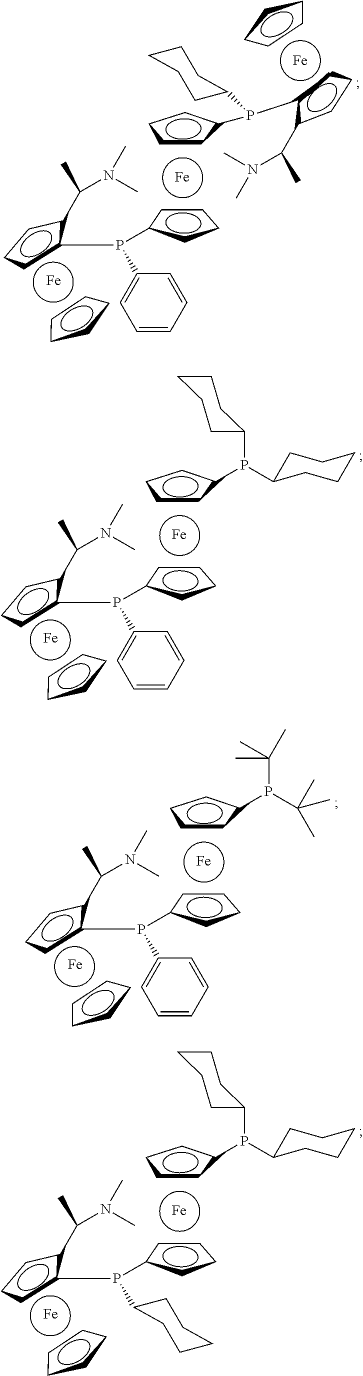 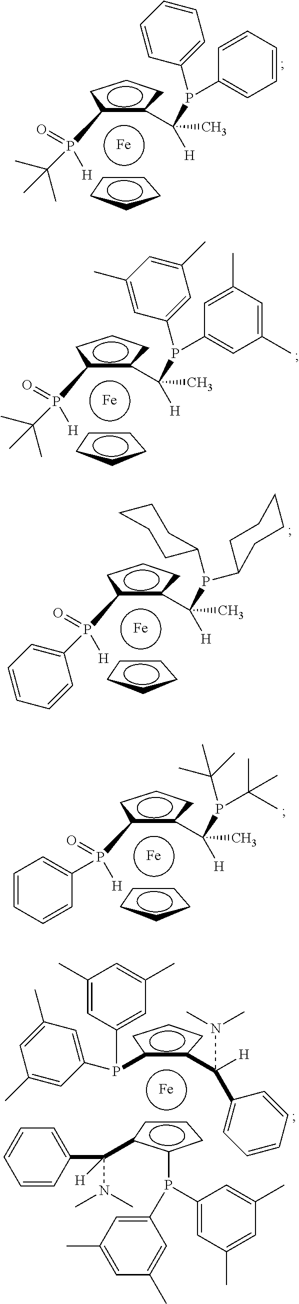 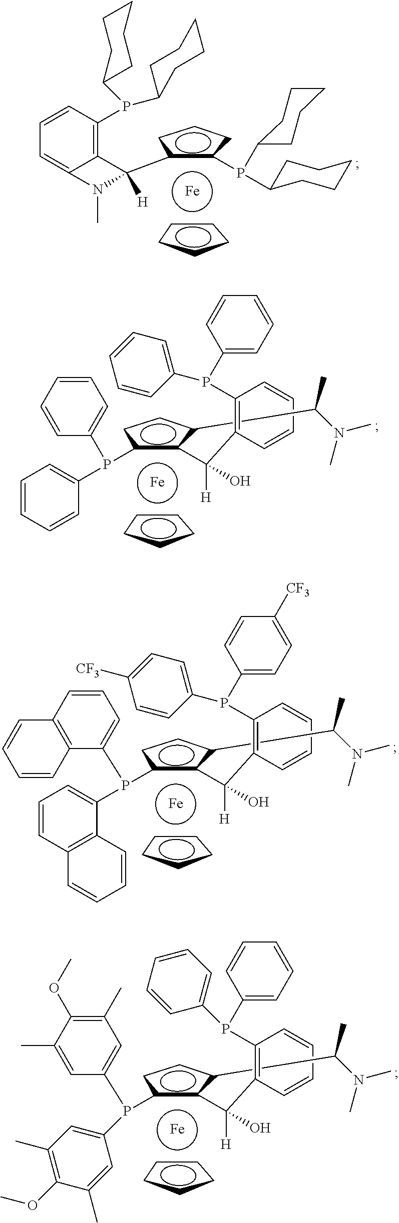 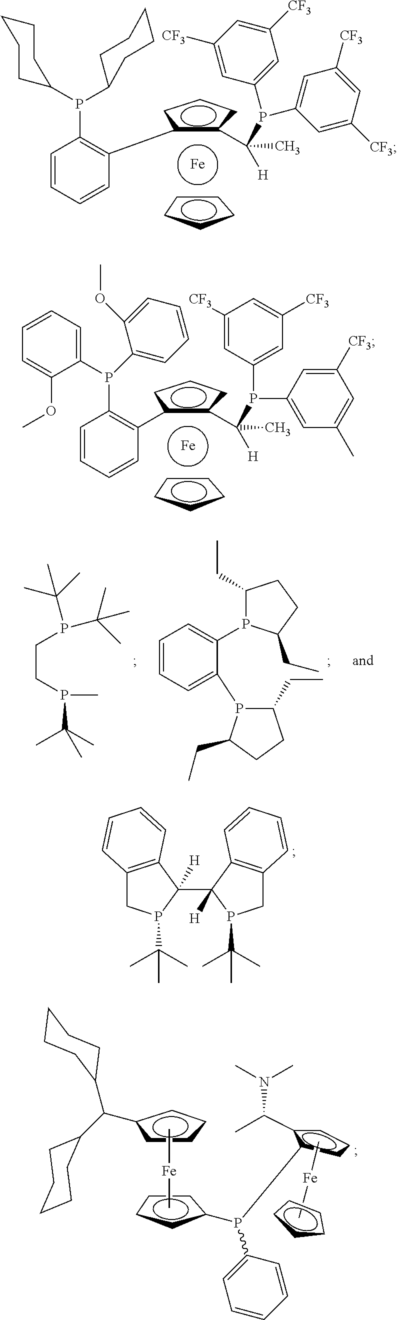 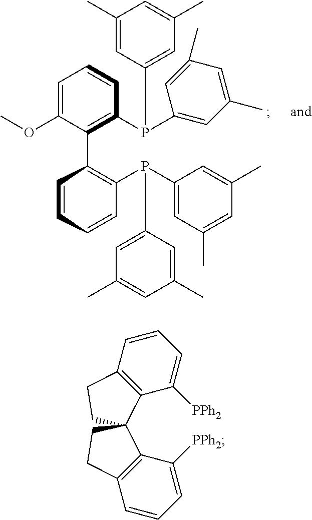
or the enantiomer thereof.

13. The process according to claim 11, wherein the chiral tertiary diphosphine has the following chemical formula: 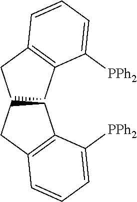
or the enantiomer thereof.

14. The process according to claim 11, wherein the chiral tertiary diphosphine has the following chemical formula: 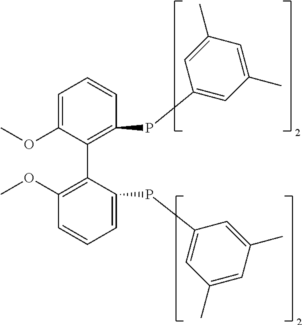
or the enantiomer thereof.

15. The process according to claim 11, wherein the chiral tertiary diphosphine has the following chemical formula: 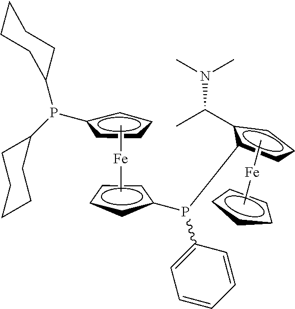
or the enantiomer thereof.

16. The process according to claim 6, wherein the chiral phosphorus-containing ligand is a chiral tertiary diphosphine.

17. The process according to claim 16, wherein the chiral tertiary diphosphine has the following chemical formula: 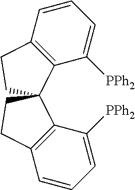
or the enantiomer thereof.

18. The process according to claim 16, wherein the chiral tertiary diphosphine has the following chemical formula: 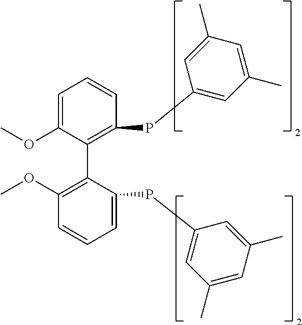
or the enantiomer thereof.

19. The process according to claim 16, wherein the chiral tertiary diphosphine has the following chemical formula: 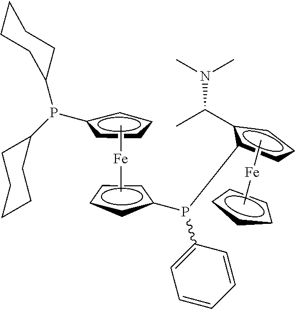
or the enantiomer thereof.

20. A compound of the Formula IIa′: 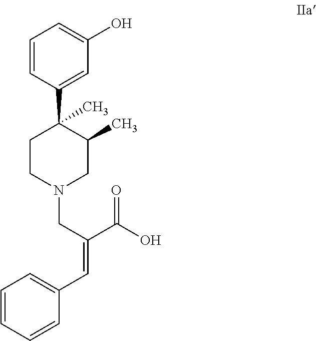 [SMILES: C[C@H]1CN(C/C(=C\c2ccccc2)C(=O)O)CC[C@@]1(C)c1cccc(O)c1]
or a pharmaceutically acceptable salt thereof.

21. A compound of the Formula IIb′: 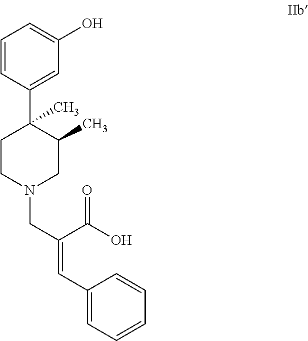 [SMILES: C[C@H]1CN(C/C(=C/c2ccccc2)C(=O)O)CC[C@@]1(C)c1cccc(O)c1]
or a pharmaceutically acceptable salt thereof.
